(19) United States
(12) Patent Application Publication
(10) Pub. No.: US 2022/0401438 A1
(43) Pub. Date: Dec. 22, 2022

(54) CANCER THERAPEUTIC METHODS

(71) Applicant: University Health Network, Toronto (CA)

(72) Inventors: Anthony Michael JOSHUA, Toronto (CA); Marianne KORITZINSKY, Toronto (CA); Sue LI, Toronto (CA); Stefano MARASTONI, Toronto (CA); Bradley G. WOUTERS, Toronto (CA)

(21) Appl. No.: 17/727,394

(22) Filed: Apr. 22, 2022

(30) Foreign Application Priority Data
Apr. 22, 2021 (AU) 2021202471
Apr. 22, 2021 (CA) 3116081

(51) Int. Cl. A61K 31/496; A61K 31/4706; A61K 31/138; A61P 35/00

(52) U.S. Cl. CPC A61K 31/496; A61K 31/4706; A61K 31/138; A61P 35/00

(57) ABSTRACT

Described herein are methods and compositions for screening cancer cells for sensitivity to itraconazole and use of itraconazole in treatment of cancer.

### PRIORITY CLAIM

This application claims benefit of priority to Canadian Patent Application Serial No. 3,116,081, filed Apr. 22, 2021 and Australian Patent Application No. 2021202471, filed Apr. 22, 2021, the entire contents of which are hereby incorporated by reference.

### SEQUENCE LISTING

Pursuant to 37 C.F.R. 1.821(c), a sequence listing is submitted herewith as an ASCII compliant text file named “SWIP.P0004US.txt”, created on Apr. 22, 2022 and having a size of ˜6 kilobytes. The content of the aforementioned file is hereby incorporated by reference in its entirety.

### FIELD OF THE INVENTION

The invention relates to cancer, including but not limited to ovarian cancer and to itraconazole and therapeutic uses of same.

### BACKGROUND OF THE INVENTION

Epithelial ovarian cancer (EOC) is the most lethal gynecologic malignancy in developed countries¹. It can be divided into five subtypes—high and low grade serous, endometrioid, clear cell and mucinous carcinomas². Treatment for advanced EOC is multi-modal integrating surgery and platinum-based chemotherapy, which are tailored according to histological subtype, stage and patient characteristics. Contemporary systemic treatments in recurrent EOC include chemotherapy, anti-angiogenic agents and poly(ADP-ribose) polymerase (PARP) inhibitors³. Platinum resistant EOC—defined as disease progression occurring in <6 months from last dose of platinum—remains an aggressive disease with limited effective treatment options and response to chemotherapy (approximately 10-15%)^{3,4}.

An emerging strategy to expand the therapeutic armamentarium against ovarian cancer is drug repurposing. A widely used antifungal drug, Itraconazole (Itra) has been investigated in several cancer types⁵⁻⁷. Current understanding of the mechanism of action of Itra in cancer include: i) reversing chemoresistance mediated by P-glycoprotein;⁸ ii) potent anti-angiogenic and anti-lymphangiogenic activity;⁹⁻¹¹ iii) modulating Hedgehog (Hh) and Wnt/ß catenin signaling;¹²⁻¹⁵ iv) inhibiting the mitochondrial protein voltage-dependent anion channel 1 (VDAC1);¹⁶ and v) altering cholesterol trafficking by direct binding and inhibition of the lysosomal protein and cholesterol transporter Niemann-Pick disease, type C1 (NPC1)¹⁷. These latter two effects have been demonstrated to result in inhibition of the mTOR (mammalian target of rapamycin) pathway¹⁸. A number of preclinical and clinical studies have suggested that patients with prostate, lung, and basal cell carcinoma may benefit from treatment with Itra monotherapy or in combination with chemotherapy^{5-7,19-21}. In platinum resistant EOC, two retrospective studies in patients with recurrent ovarian cancer have demonstrated the beneficial effects of Itra in combination with taxane-based chemotherapy^{22,23}.

Investigations to date have revealed that itraconazole has varying efficacy for treatment of cancer from individual to individual^{5-7,19-23}.

To the inventor's knowledge, presently there is no method for enabling one to identify an individual in which itraconazole is likely to have some efficacy for treatment of cancer, especially ovarian cancer, nor to identify an individual in which intraconazole is likely to have limited or no efficacy for treatment of cancer, especially ovarian cancer.

There is a need for methods and compositions for providing improvements in the treatment of cancer, especially ovarian cancer.

There is a need for methods and compositions for providing improvements in the use of itraconazole to treat cancer, especially ovarian cancer.

There is a need for methods and compositions for providing improvements in identifying individuals in which itraconazole is likely to have at least some efficacy for the treatment of cancer, especially for the treatment of ovarian cancer.

### SUMMARY OF THE INVENTION

The invention relates to methods of treating, preventing, or ameliorating cancer, especially ovarian cancer, or conditions associated with, or arising from same, and to pharmaceutical compositions and kits comprising itraconazole for use in an individual for treating or preventing cancer, especially ovarian cancer or conditions associated with, or arising from same.

The invention provides a method for preventing or treating cancer or a condition associated with same in an individual comprising providing a therapeutically effective amount of itraconazole in an individual, wherein the individual has a cancer cell having a mutation, preferably a loss of function mutation in a gene selected from the group consisting of: NBEAL1, C18orf8, COG6, SLC30A9, ARL1, UNC50, EPT1, RTN4IP1, SPTLC2, SEC14L1, CCDC22, SPTSSA, KIAA1033, SPTLC1, VPS54, TSSC1, KIF11, NDUFS4, COMMD4, COL4A3BP, VPS51, VPS52 and VPS53, or wherein the individual has a cancer cell having a reduced production or no production of a protein or RNA encoded by a gene selected from said group.

The invention further provides a composition comprising a therapeutically effective amount of itraconazole for use in preventing or treating cancer or a condition associated with same in an individual, wherein the individual has a cancer cell having a mutation, preferably a loss of function mutation in a gene selected from the group consisting of: NBEAL1, C18orf8, COG6, SLC30A9, ARL1, UNC50, EPT1, RTN4IP1, SPTLC2, SEC14L1, CCDC22, SPTSSA, KIAA1033, SPTLC1, VPS54, TSSC1, KIF11, NDUFS4, COMMD4, COL4A3BP, VPS51, VPS52 and VPS53, or wherein the individual has a cancer cell having a reduced production or no production of a protein or RNA encoded by a gene selected from said group.

The invention further provides a use of a composition comprising itraconazole in the manufacture of a medicament for preventing or treating cancer or a condition associated with same in an individual, wherein the individual has a cancer cell having a mutation, preferably a loss of function mutation in a gene selected from the group consisting of: NBEAL1, C18orf8, COG6, SLC30A9, ARL1, UNC50, EPT1, RTN4IP1, SPTLC2, SEC14L1, CCDC22, SPTSSA, KIAA1033, SPTLC1, VPS54, TSSC1, KIF11, NDUFS4, COMMD4, COL4A3BP, VPS51, VPS52 and VPS53, or wherein the individual has a cancer cell having a reduced production or no production of a protein or RNA encoded by a gene selected from said group.

The invention further provides itraconazole when used for preventing or treating cancer or a condition associated with same in an individual, wherein the individual has a cancer cell having a mutation, preferably a loss of function mutation in a gene selected from the group consisting of: NBEAL1, C18orf8, COG6, SLC30A9, ARL1, UNC50, EPT1, RTN4IP1, SPTLC2, SEC14L1, CCDC22, SPTSSA, KIAA1033, SPTLC1, VPS54, TSSC1, KIF11, NDUFS4, COMMD4, COL4A3BP, VPS51, VPS52 and VPS53, or wherein the individual has a cancer cell having a reduced production or no production of a protein or RNA encoded by a gene selected from said group.

The invention further provides a method for preventing or treating cancer or condition associated with same in an individual comprising:
assessing or having assessed, a sample comprising a cell, preferably a cancer cell obtained from an individual for whom cancer is to be prevented or treated to determine if a cell in the sample has a mutation, preferably a loss of function mutation in a gene selected from the group consisting of: NBEAL1, C18orf8, COG6, SLC30A9, ARL1, UNC50, EPT1, RTN4IP1, SPTLC2, SEC14L1, CCDC22, SPTSSA, KIAA1033, SPTLC1, VPS54, TSSC1, KIF11, NDUFS4, COMMD4, COL4A3BP, VPS51, VPS52 and VPS53; and
where the individual has a cell having a mutation, preferably a loss of function mutation in a gene selected from the group consisting of: NBEAL1, Cl 8orf8, COG6, SLC30A9, ARL1, UNC50, EPT1, RTN4IP1, SPTLC2, SEC14L1, CCDC22, SPTSSA, KIAA1033, SPTLC1, VPS54, TSSC1, KIF11, NDUFS4, COMMD4, COL4A3BP, VPS51, VPS52 and VPS53,
   providing itraconazole to the individual,
      thereby preventing or treating cancer or condition associated with same in the individual.

The invention further provides a method for determining the likelihood of an individual presenting an anti-cancer response to administration of itraconazole comprising:
assessing or having assessed, a sample, preferably a sample of cancer cells obtained from an individual for whom the likelihood of presenting an anti-cancer response to administration of itraconazole is to be determined to determine whether a cancer cell in the sample has a mutation, preferably a loss of function mutation in a gene selected from the group consisting of: NBEAL1, C18orf8, COG6, SLC30A9, ARL1, UNC50, EPT1, RTN4IP1, SPTLC2, SEC14L1, CCDC22, SPTSSA, KIAA1033, SPTLC1, VPS54, TSSC1, KIF11, NDUFS4, COMMD4, COL4A3BP, VPS51, VPS52 and VPS53;
determining that the individual has a higher likelihood of presenting an anti-cancer response to administration of itraconazole where a cancer cell in the sample has a mutation, preferably a loss of function mutation in a gene selected from the group consisting of: NBEAL1, Cl 8orf8, COG6, SLC30A9, ARL1, UNC50, EPT1, RTN4IP1, SPTLC2, SEC14L1, CCDC22, SPTSSA, KIAA1033, SPTLC1, VPS54, TSSC1, KIF11, NDUFS4, COMMD4, COL4A3BP, VPS51, VPS52 and VPS53;
determining that the individual has a lower likelihood of presenting an anti-cancer response to administration of itraconazole where a cancer cell in the sample does not have a mutation, preferably a loss of function mutation in a gene selected from the group consisting of: NBEAL1, C18orf8, COG6, SLC30A9, ARL1, UNC50, EPT1, RTN4IP1, SPTLC2, SEC14L1, CCDC22, SPTSSA, KIAA1033, SPTLC1, VPS54, TSSC1, KIF11, NDUFS4, COMMD4, COL4A3BP, VPS51, VPS52 and VPS53;
   thereby determining the likelihood of an individual presenting an anti-cancer response to administration of itraconazole.

The invention further provides a method for selecting an individual for treatment of cancer with itraconazole comprising the step of:
assessing or having assessed a cell of the individual, preferably a cancer cell to determine if the cell has a mutation, preferably a loss of function mutation in a gene selected from the group consisting of: NBEAL1, Cl 8orf8, COG6, SLC30A9, ARL1, UNC50, EPT1, RTN4IP1, SPTLC2, SEC14L1, CCDC22, SPTSSA, KIAA1033, SPTLC1, VPS54, TSSC1, KIF11, NDUFS4, COMMD4, COL4A3BP, VPS51, VPS52 and VPS53;
selecting the individual for treatment of cancer with itraconazole where the individual has a mutation, preferably a loss of function mutation in a gene selected from the group consisting of: NBEAL1, Cl 8orf8, COG6, SLC30A9, ARL1, UNC50, EPT1, RTN4IP1, SPTLC2, SEC14L1, CCDC22, SPTSSA, KIAA1033, SPTLC1, VPS54, TSSC1, KIF11, NDUFS4, COMMD4, COL4A3BP, VPS51, VPS52 and VPS53;
   thereby selecting an individual for treatment of cancer with itraconazole.

The invention further provides a method for administration of itraconazole to an individual requiring therapy for cancer comprising the step of:
assessing or having assessed a cell of the individual, preferably a cancer cell to determine if the cell has a mutation, preferably a loss of function mutation in a gene selected from the group consisting of: NBEAL1, Cl 8orf8, COG6, SLC30A9, ARL1, UNC50, EPT1, RTN4IP1, SPTLC2, SEC14L1, CCDC22, SPTSSA, KIAA1033, SPTLC1, VPS54, TSSC1, KIF11, NDUFS4, COMMD4, COL4A3BP, VPS51, VPS52 and VPS53;
   optionally
administering a therapeutically effective amount of itraconazole to the individual for therapy of cancer.

The invention further provides a method for treatment or prevention of cancer comprising providing in an individual requiring said treatment
itraconazole
a lysosomotropic compound, preferably selected from the group consisting of chloroquine, azithromycin, dririthromycin, clarithromycin, erithromycin, promethazine, cyclizine, amodiaquine, telithromycin, tripelennamine, clemastine fumarate, hydroxyzine, rifabutin, mefloquine, chlorcyclyzine, homochlorcyclzine, quinacrine, meclizine, tilorone, bepotastine besilate, vicriviroc, hydroxychloroquine, quinine, arbidol, nelfinavir and tamoxifen,

preferably wherein the itraconazole and lyosomotropic compound are provided in the individual in synergistically effective amounts;

preferably wherein the cancer of the individual has a mutation, preferably a loss of function mutation in a gene selected from the group consisting of NBEAL1, Cl 8orf8, COG6, SLC30A9, ARL1, UNC50, EPT1, RTN4IP1, SPTLC2, SEC14L1, CCDC22, SPTSSA, KIAA1033, SPTLC1, VPS54, TSSC1, KIF11, NDUFS4, COMMD4, COL4A3BP, VPS51, VPS52 and VPS53.

The above described methods may comprise the step of determining whether the cell of the individual has reduced production, or no production of a protein or RNA encoded by a gene selected from the group consisting of: NBEAL1; Cl 8orf8, COG6, SLC30A9, ARL1, UNC50, EPT1, RTN4IP1, SPTLC2, SEC14L1, CCDC22, SPTSSA, KIAA1033, SPTLC1, VPS54, TSSC1, KIF11, NDUFS4, COMMD4, COL4A3BP, VPS51, VPS52 and VPS53, wherein reduced production or no production of a protein or RNA encoded by a gene selected from said group determines that the cell has a loss of function mutation in the gene, thereby determining whether the cell has a loss of function mutation in a gene of said group.

The invention further provides a kit comprising:
a probe for assessing for presence or absence of a mutation, preferably a loss of function mutation in a gene selected from the group consisting of NBEAL1, C18orf8, COG6, SLC30A9, ARL1, UNC50, EPT1, RTN4IP1, SPTLC2, SEC14L1, CCDC22, SPTSSA, KIAA1033, SPTLC1, VPS54, TSSC1, KIF11, NDUFS4, COMMD4, COL4A3BP, VPS51, VPS52 and VPS53; or
a probe for assessing for reduced production or no production of a protein or RNA encoded by a gene selected from said group;
written instructions for use of the kit in an enumerated embodiment described below.

Various (enumerated) embodiments of the present invention are described herein. It will be recognized that features specified in each embodiment may be combined with other specified features to provide further embodiments of the present disclosure.

Embodiment 1: A method for preventing or treating cancer, preferably ovarian cancer, more preferably platinum resistant or refractory epithelilal ovarian cancer in an individual, comprising
administering a therapeutically effective amount of itraconazole to the individual wherein a cancer cell of the individual has a mutation, preferably a loss of function mutation in NBEAL1 (SEQ ID No:1); more preferably a loss of function mutation in NBEAL1 (SEQ ID No:1) and a mutation in a gene selected from the group consisting of C18orf8 (SEQ ID No:2), COG6 (SEQ ID No:3), SLC30A9 (SEQ ID No:4), ARL1 (SEQ ID No:5), UNC50 (SEQ ID No:6), EPT1 (SEQ ID No:7), RTN4IP1 (SEQ ID No:8), SPTLC2 (SEQ ID No:9), SEC14L1 (SEQ ID No:10), CCDC22 (SEQ ID No:11), SPTSSA (SEQ ID No:12), KIAA1033 (SEQ ID No:13), SPTLC1 (SEQ ID No:14), VPS54 (SEQ ID No:15), TSSC1 (SEQ ID No:16), KIF11 (SEQ ID No:17), NDUFS4 (SEQ ID No:18), COMMD4 (SEQ ID No:19), COL4A3BP (SEQ ID No:20), VPS51 (SEQ ID No:21), VPS52 (SEQ ID No:22) and VPS53 (SEQ ID No:23), more preferably a loss of function mutation in NBEAL1 (SEQ ID No:1) and a mutation in a gene selected from the group consisting of C18orf8 and VPS54,
optionally comprising the further step of providing in the individual a lysosomotropic compound, preferably chloroquine, azithromycin, dririthromycin, clarithromycin, erithromycin, promethazine, cyclizine, amodiaquine, telithromycin, tripelennamine, clemastine fumarate, hydroxyzine, rifabutin, mefloquine, chlorcyclyzine, homochlorcyclzine, quinacrine, meclizine, tilorone, bepotastine besilate, vicriviroc, hydroxychloroquine, quinine, arbidol, nelfinavir or tamoxifen, wherein the lysosomotropic compound and itraconazole are provided in synergistically effective amounts.

Embodiment 2: A method for preventing or treating cancer, preferably ovarian cancer, more preferably platinum resistant or refractory epithelilal ovarian cancer in an individual, comprising
administering a therapeutically effective amount of itraconazole to the individual wherein a cancer cell of the individual has a mutation, preferably a loss of function mutation in C18orf8 (SEQ ID No:2); more preferably a loss of function mutation in C18orf8 (SEQ ID No:2) and a mutation in a gene selected from the group consisting of NBEAL1 (SEQ ID No:1), COG6 (SEQ ID No:3), SLC30A9 (SEQ ID No:4), ARL1 (SEQ ID No:5), UNC50 (SEQ ID No:6), EPT1 (SEQ ID No:7), RTN4IP1 (SEQ ID No:8), SPTLC2 (SEQ ID No:9), SEC14L1 (SEQ ID No:10), CCDC22 (SEQ ID No:11), SPTSSA (SEQ ID No:12), KIAA1033 (SEQ ID No:13), SPTLC1 (SEQ ID No:14), VPS54 (SEQ ID No:15), TSSC1 (SEQ ID No:16), KIF11 (SEQ ID No:17), NDUFS4 (SEQ ID No:18), COMMD4 (SEQ ID No:19), COL4A3BP (SEQ ID No:20), VPS51 (SEQ ID No:21), VPS52 (SEQ ID No:22) and VPS53 (SEQ ID No:23), more preferably a loss of function mutation in C18orf8 (SEQ ID No:2) and a mutation in gene VPS54,
optionally comprising the further step of providing in the individual a lysosomotropic compound, preferably chloroquine, azithromycin, dririthromycin, clarithromycin, erithromycin, promethazine, cyclizine, amodiaquine, telithromycin, tripelennamine, clemastine fumarate, hydroxyzine, rifabutin, mefloquine, chlorcyclyzine, homochlorcyclzine, quinacrine, meclizine, tilorone, bepotastine besilate, vicriviroc, hydroxychloroquine, quinine, arbidol, nelfinavir or tamoxifen, wherein the lysosomotropic compound and itraconazole are provided in synergistically effective amounts.

Embodiment 3: A method for preventing or treating cancer, preferably ovarian cancer, more preferably platinum resistant or refractory epithelilal ovarian cancer in an individual, comprising
administering a therapeutically effective amount of itraconazole to the individual wherein a cancer cell of the individual has a mutation, preferably a loss of function mutation in COG6 (SEQ ID No:3); more preferably a loss of function mutation in COG6 (SEQ ID No:3) and a mutation in a gene selected from the group consisting of NBEAL1 (SEQ ID No:1), C18orf8 (SEQ ID No:2), SLC30A9 (SEQ ID No:4), ARL1 (SEQ ID No:5), UNC50 (SEQ ID No:6), EPT1 (SEQ ID No:7), RTN4IP1 (SEQ ID No:8), SPTLC2 (SEQ ID No:9), SEC14L1 (SEQ ID No:10), CCDC22 (SEQ ID No:11), SPTSSA (SEQ ID No:12), KIAA1033 (SEQ ID No:13), SPTLC1 (SEQ ID No:14), VPS54 (SEQ ID No:15), TSSC1 (SEQ ID No:16), KIF11 (SEQ ID No:17), NDUFS4 (SEQ ID No:18), COMMD4 (SEQ ID No:19), COL4A3BP (SEQ ID No:20), VPS51 (SEQ ID No:21), VPS52 (SEQ ID No:22) and VPS53 (SEQ ID No:23), more preferably a loss of function mutation in COG6 (SEQ ID No:3) and a mutation in a gene selected from the group consisting of C18orf8 and VPS54,
optionally comprising the further step of providing in the individual a lysosomotropic compound, preferably chloroquine, azithromycin, dririthromycin, clarithromycin, erithromycin, promethazine, cyclizine, amodiaquine, telithromycin, tripelennamine, clemastine fumarate, hydroxyzine, rifabutin, mefloquine, chlorcyclyzine, homochlorcyclzine, quinacrine, meclizine, tilorone, bepotastine besilate, vicriviroc, hydroxychloroquine, quinine, arbidol, nelfinavir or tamoxifen, wherein the lysosomotropic compound and itraconazole are provided in synergistically effective amounts.

Embodiment 4: A method for preventing or treating cancer, preferably ovarian cancer, more preferably platinum resistant or refractory epithelilal ovarian cancer in an individual, comprising
administering a therapeutically effective amount of itraconazole to the individual wherein a cancer cell of the individual has a mutation, preferably a loss of function mutation in SLC30A9 (SEQ ID No:4); more preferably a loss of function mutation in SLC30A9 (SEQ ID No:4) and a mutation in a gene selected from the group consisting of NBEAL1 (SEQ ID No:1), C18orf8 (SEQ ID No:2), COG6 (SEQ ID No:3), ARL1 (SEQ ID No:5), UNC50 (SEQ ID No:6), EPT1 (SEQ ID No:7), RTN4IP1 (SEQ ID No:8), SPTLC2 (SEQ ID No:9), SEC14L1 (SEQ ID No:10), CCDC22 (SEQ ID No:11), SPTSSA (SEQ ID No:12), KIAA1033 (SEQ ID No:13), SPTLC1 (SEQ ID No:14), VPS54 (SEQ ID No:15), TSSC1 (SEQ ID No:16), KIF11 (SEQ ID No:17), NDUFS4 (SEQ ID No:18), COMMD4 (SEQ ID No:19), COL4A3BP (SEQ ID No:20), VPS51 (SEQ ID No:21), VPS52 (SEQ ID No:22) and VPS53 (SEQ ID No:23), more preferably a loss of function mutation in SLC30A9 (SEQ ID No:4) and a mutation in a gene selected from the group consisting of C18orf8 and VPS54,
optionally comprising the further step of providing in the individual a lysosomotropic compound, preferably chloroquine, azithromycin, dririthromycin, clarithromycin, erithromycin, promethazine, cyclizine, amodiaquine, telithromycin, tripelennamine, clemastine fumarate, hydroxyzine, rifabutin, mefloquine, chlorcyclyzine, homochlorcyclzine, quinacrine, meclizine, tilorone, bepotastine besilate, vicriviroc, hydroxychloroquine, quinine, arbidol, nelfinavir or tamoxifen, wherein the lysosomotropic compound and itraconazole are provided in synergistically effective amounts.

Embodiment 5: A method for preventing or treating cancer, preferably ovarian cancer, more preferably platinum resistant or refractory epithelilal ovarian cancer in an individual, comprising
administering a therapeutically effective amount of itraconazole to the individual wherein a cancer cell of the individual has a mutation, preferably a loss of function mutation in ARL1 (SEQ ID No:5); more preferably a loss of function mutation in ARL1 (SEQ ID No:5) and a mutation in a gene selected from the group consisting of NBEAL1 (SEQ ID No:1), C18orf8 (SEQ ID No:2), COG6 (SEQ ID No:3), SLC30A9 (SEQ ID No:4), UNC50 (SEQ ID No:6), EPT1 (SEQ ID No:7), RTN4IP1 (SEQ ID No:8), SPTLC2 (SEQ ID No:9), SEC14L1 (SEQ ID No:10), CCDC22 (SEQ ID No:11), SPTSSA (SEQ ID No:12), KIAA1033 (SEQ ID No:13), SPTLC1 (SEQ ID No:14), VPS54 (SEQ ID No:15), TSSC1 (SEQ ID No:16), KIF11 (SEQ ID No:17), NDUFS4 (SEQ ID No:18), COMMD4 (SEQ ID No:19), COL4A3BP (SEQ ID No:20), VPS51 (SEQ ID No:21), VPS52 (SEQ ID No:22) and VPS53 (SEQ ID No:23), more preferably a loss of function mutation in ARL1 (SEQ ID No:5) and a mutation in a gene selected from the group consisting of C18orf8 and VPS54,
optionally comprising the further step of providing in the individual a lysosomotropic compound, preferably chloroquine, azithromycin, dririthromycin, clarithromycin, erithromycin, promethazine, cyclizine, amodiaquine, telithromycin, tripelennamine, clemastine fumarate, hydroxyzine, rifabutin, mefloquine, chlorcyclyzine, homochlorcyclzine, quinacrine, meclizine, tilorone, bepotastine besilate, vicriviroc, hydroxychloroquine, quinine, arbidol, nelfinavir or tamoxifen, wherein the lysosomotropic compound and itraconazole are provided in synergistically effective amounts.

Embodiment 6: A method for preventing or treating cancer, preferably ovarian cancer, more preferably platinum resistant or refractory epithelilal ovarian cancer in an individual, comprising
administering a therapeutically effective amount of itraconazole to the individual wherein a cancer cell of the individual has a mutation, preferably a loss of function mutation in UNC50 (SEQ ID No:6); more preferably a loss of function mutation in UNC50 (SEQ ID No:6) and a mutation in a gene selected from the group consisting of NBEAL1 (SEQ ID No:1), C18orf8 (SEQ ID No:2), COG6 (SEQ ID No:3), SLC30A9 (SEQ ID No:4), ARL1 (SEQ ID No:5), EPT1 (SEQ ID No:7), RTN4IP1 (SEQ ID No:8), SPTLC2 (SEQ ID No:9), SEC14L1 (SEQ ID No:10), CCDC22 (SEQ ID No:11), SPTSSA (SEQ ID No:12), KIAA1033 (SEQ ID No:13), SPTLC1 (SEQ ID No:14), VPS54 (SEQ ID No:15), TSSC1 (SEQ ID No:16), KIF11 (SEQ ID No:17), NDUFS4 (SEQ ID No:18), COMMD4 (SEQ ID No:19), COL4A3BP (SEQ ID No:20), VPS51 (SEQ ID No:21), VPS52 (SEQ ID No:22) and VPS53 (SEQ ID No:23), more preferably a loss of function mutation in UNC50 (SEQ ID No:6) and a mutation in a gene selected from the group consisting of C18orf8 and VPS54,
optionally comprising the further step of providing in the individual a lysosomotropic compound, preferably chloroquine, azithromycin, dririthromycin, clarithromycin, erithromycin, promethazine, cyclizine, amodiaquine, telithromycin, tripelennamine, clemastine fumarate, hydroxyzine, rifabutin, mefloquine, chlorcyclyzine, homochlorcyclzine, quinacrine, meclizine, tilorone, bepotastine besilate, vicriviroc, hydroxychloroquine, quinine, arbidol, nelfinavir or tamoxifen, wherein the lysosomotropic compound and itraconazole are provided in synergistically effective amounts.

Embodiment 7: A method for preventing or treating cancer, preferably ovarian cancer, more preferably platinum resistant or refractory epithelilal ovarian cancer in an individual, comprising
administering a therapeutically effective amount of itraconazole to the individual wherein a cancer cell of the individual has a mutation, preferably a loss of function mutation in EPT1 (SEQ ID No:7); more preferably a loss of function mutation in EPT1 (SEQ ID No:7) and a mutation in a gene selected from the group consisting of NBEAL1 (SEQ ID No:1), C18orf8 (SEQ ID No:2), COG6 (SEQ ID No:3), SLC30A9 (SEQ ID No:4), ARL1 (SEQ ID No:5), UNC50 (SEQ ID No:6), RTN4IP1 (SEQ ID No:8), SPTLC2 (SEQ ID No:9), SEC14L1 (SEQ ID No:10), CCDC22 (SEQ ID No:11), SPTSSA (SEQ ID No:12), KIAA1033 (SEQ ID No:13), SPTLC1 (SEQ ID No:14), VPS54 (SEQ ID No:15), TSSC1 (SEQ ID No:16), KIF11 (SEQ ID No:17), NDUFS4 (SEQ ID No:18), COMMD4 (SEQ ID No:19), COL4A3BP (SEQ ID No:20), VPS51 (SEQ ID No:21), VPS52 (SEQ ID No:22) and VPS53 (SEQ ID No:23), more preferably a loss of function mutation in EPT1 (SEQ ID No:7) and a mutation in a gene selected from the group consisting of C18orf8 and VPS54,
optionally comprising the further step of providing in the individual a lysosomotropic compound, preferably chloroquine, azithromycin, dririthromycin, clarithromycin, erithromycin, promethazine, cyclizine, amodiaquine, telithromycin, tripelennamine, clemastine fumarate, hydroxyzine, rifabutin, mefloquine, chlorcyclyzine, homochlorcyclzine, quinacrine, meclizine, tilorone, bepotastine besilate, vicriviroc, hydroxychloroquine, quinine, arbidol, nelfinavir or tamoxifen, wherein the lysosomotropic compound and itraconazole are provided in synergistically effective amounts.

Embodiment 8: A method for preventing or treating cancer, preferably ovarian cancer, more preferably platinum resistant or refractory epithelilal ovarian cancer in an individual, comprising
administering a therapeutically effective amount of itraconazole to the individual wherein a cancer cell of the individual has a mutation, preferably a loss of function mutation in RTN4IP1 (SEQ ID No:8); more preferably a loss of function mutation in RTN4IP1 (SEQ ID No:8) and a mutation in a gene selected from the group consisting of NBEAL1 (SEQ ID No:1), C18orf8 (SEQ ID No:2), COG6 (SEQ ID No:3), SLC30A9 (SEQ ID No:4), ARL1 (SEQ ID No:5), UNC50 (SEQ ID No:6), EPT1 (SEQ ID No:7), SPTLC2 (SEQ ID No:9), SEC14L1 (SEQ ID No:10), CCDC22 (SEQ ID No:11), SPTSSA (SEQ ID No:12), KIAA1033 (SEQ ID No:13), SPTLC1 (SEQ ID No:14), VPS54 (SEQ ID No:15), TSSC1 (SEQ ID No:16), KIF11 (SEQ ID No:17), NDUFS4 (SEQ ID No:18), COMMD4 (SEQ ID No:19), COL4A3BP (SEQ ID No:20), VPS51 (SEQ ID No:21), VPS52 (SEQ ID No:22) and VPS53 (SEQ ID No:23), more preferably a loss of function mutation in RTN4IP1 (SEQ ID No:8) and a mutation in a gene selected from the group consisting of C18orf8 and VPS54,
optionally comprising the further step of providing in the individual a lysosomotropic compound, preferably chloroquine, azithromycin, dririthromycin, clarithromycin, erithromycin, promethazine, cyclizine, amodiaquine, telithromycin, tripelennamine, clemastine fumarate, hydroxyzine, rifabutin, mefloquine, chlorcyclyzine, homochlorcyclzine, quinacrine, meclizine, tilorone, bepotastine besilate, vicriviroc, hydroxychloroquine, quinine, arbidol, nelfinavir or tamoxifen, wherein the lysosomotropic compound and itraconazole are provided in synergistically effective amounts.

Embodiment 9: A method for preventing or treating cancer, preferably ovarian cancer, more preferably platinum resistant or refractory epithelilal ovarian cancer in an individual, comprising
administering a therapeutically effective amount of itraconazole to the individual wherein a cancer cell of the individual has a mutation, preferably a loss of function mutation in SPTLC2 (SEQ ID No:9); more preferably a loss of function mutation in SPTLC2 (SEQ ID No:9) and a mutation in a gene selected from the group consisting of NBEAL1 (SEQ ID No:1); C18orf8 (SEQ ID No:2), COG6 (SEQ ID No:3), SLC30A9 (SEQ ID No:4), ARL1 (SEQ ID No:5), UNC50 (SEQ ID No:6), EPT1 (SEQ ID No:7), RTN4IP1 (SEQ ID No:8), SEC14L1 (SEQ ID No:10), CCDC22 (SEQ ID No:11), SPTSSA (SEQ ID No:12), KIAA1033 (SEQ ID No:13), SPTLC1 (SEQ ID No:14), VPS54 (SEQ ID No:15), TSSC1 (SEQ ID No:16), KIF11 (SEQ ID No:17), NDUFS4 (SEQ ID No:18), COMMD4 (SEQ ID No:19), COL4A3BP (SEQ ID No:20), VPS51 (SEQ ID No:21), VPS52 (SEQ ID No:22) and VPS53 (SEQ ID No:23), more preferably a loss of function mutation in SPTLC2 (SEQ ID No:9) and a mutation in a gene selected from the group consisting of C18orf8 and VPS54,
optionally comprising the further step of providing in the individual a lysosomotropic compound, preferably chloroquine, azithromycin, dririthromycin, clarithromycin, erithromycin, promethazine, cyclizine, amodiaquine, telithromycin, tripelennamine, clemastine fumarate, hydroxyzine, rifabutin, mefloquine, chlorcyclyzine, homochlorcyclzine, quinacrine, meclizine, tilorone, bepotastine besilate, vicriviroc, hydroxychloroquine, quinine, arbidol, nelfinavir or tamoxifen, wherein the lysosomotropic compound and itraconazole are provided in synergistically effective amounts.

Embodiment 10: A method for preventing or treating cancer, preferably ovarian cancer, more preferably platinum resistant or refractory epithelilal ovarian cancer in an individual, comprising
administering a therapeutically effective amount of itraconazole to the individual wherein a cancer cell of the individual has a mutation, preferably a loss of function mutation in SEC14L1 (SEQ ID No:10); more preferably a loss of function mutation in SEC14L1 (SEQ ID No:10) and a mutation in a gene selected from the group consisting of NBEAL1 (SEQ ID No:1); C18orf8 (SEQ ID No:2), COG6 (SEQ ID No:3), SLC30A9 (SEQ ID No:4), ARL1 (SEQ ID No:5), UNC50 (SEQ ID No:6), EPT1 (SEQ ID No:7), RTN4IP1 (SEQ ID No:8), SPTLC2 (SEQ ID No:9), CCDC22 (SEQ ID No:11), SPTSSA (SEQ ID No:12), KIAA1033 (SEQ ID No:13), SPTLC1 (SEQ ID No:14), VPS54 (SEQ ID No:15), TSSC1 (SEQ ID No:16), KIF11 (SEQ ID No:17), NDUFS4 (SEQ ID No:18), COMMD4 (SEQ ID No:19), COL4A3BP (SEQ ID No:20), VPS51 (SEQ ID No:21), VPS52 (SEQ ID No:22) and VPS53 (SEQ ID No:23), more preferably a loss of function mutation in SEC14L1 (SEQ ID No:10) and a mutation in a gene selected from the group consisting of C18orf8 and VPS54,
optionally comprising the further step of providing in the individual a lysosomotropic compound, preferably chloroquine, azithromycin, dririthromycin, clarithromycin, erithromycin, promethazine, cyclizine, amodiaquine, telithromycin, tripelennamine, clemastine fumarate, hydroxyzine, rifabutin, mefloquine, chlorcyclyzine, homochlorcyclzine, quinacrine, meclizine, tilorone, bepotastine besilate, vicriviroc, hydroxychloroquine, quinine, arbidol, nelfinavir or tamoxifen, wherein the lysosomotropic compound and itraconazole are provided in synergistically effective amounts.

Embodiment 11: A method for preventing or treating cancer, preferably ovarian cancer, more preferably platinum resistant or refractory epithelilal ovarian cancer in an individual, comprising
administering a therapeutically effective amount of itraconazole to the individual wherein a cancer cell of the individual has a mutation, preferably a loss of function mutation in CCDC22 (SEQ ID No:11); more preferably a loss of function mutation in CCDC22 (SEQ ID No:11) and a mutation in a gene selected from the group consisting of NBEAL1 (SEQ ID No:1), C18orf8 (SEQ ID No:2), COG6 (SEQ ID No:3), SLC30A9 (SEQ ID No:4), ARL1 (SEQ ID No:5), UNC50 (SEQ ID No:6), EPT1 (SEQ ID No:7), RTN4IP1 (SEQ ID No:8), SPTLC2 (SEQ ID No:9), SEC14L1 (SEQ ID No:10), SPTSSA (SEQ ID No:12), KIAA1033 (SEQ ID No:13), SPTLC1 (SEQ ID No:14), VPS54 (SEQ ID No:15), TSSC1 (SEQ ID No:16), KIF11 (SEQ ID No:17), NDUFS4 (SEQ ID No:18), COMMD4 (SEQ ID No:19), COL4A3BP (SEQ ID No:20), VPS51 (SEQ ID No:21), VPS52 (SEQ ID No:22) and VPS53 (SEQ ID No:23), more preferably a loss of function mutation in CCDC22 (SEQ ID No:11) and a mutation in a gene selected from the group consisting of C18orf8 and VPS54,
optionally comprising the further step of providing in the individual a lysosomotropic compound, preferably chloroquine, azithromycin, dririthromycin, clarithromycin, erithromycin, promethazine, cyclizine, amodiaquine, telithromycin, tripelennamine, clemastine fumarate, hydroxyzine, rifabutin, mefloquine, chlorcyclyzine, homochlorcyclzine, quinacrine, meclizine, tilorone, bepotastine besilate, vicriviroc, hydroxychloroquine, quinine, arbidol, nelfinavir or tamoxifen, wherein the lysosomotropic compound and itraconazole are provided in synergistically effective amounts.

Embodiment 12: A method for preventing or treating cancer, preferably ovarian cancer, more preferably platinum resistant or refractory epithelilal ovarian cancer in an individual, comprising
administering a therapeutically effective amount of itraconazole to the individual wherein a cancer cell of the individual has a mutation, preferably a loss of function mutation in SPTSSA (SEQ ID No:12); more preferably a loss of function mutation in SPTSSA (SEQ ID No:12) and a mutation in a gene selected from the group consisting of NBEAL1 (SEQ ID No:1), C18orf8 (SEQ ID No:2), COG6 (SEQ ID No:3), SLC30A9 (SEQ ID No:4), ARL1 (SEQ ID No:5), UNC50 (SEQ ID No:6), EPT1 (SEQ ID No:7), RTN4IP1 (SEQ ID No:8), SPTLC2 (SEQ ID No:9), SEC14L1 (SEQ ID No:10), CCDC22 (SEQ ID No:11), KIAA1033 (SEQ ID No:13), SPTLC1 (SEQ ID No:14), VPS54 (SEQ ID No:15), TSSC1 (SEQ ID No:16), KIF11 (SEQ ID No:17), NDUFS4 (SEQ ID No:18), COMMD4 (SEQ ID No:19), COL4A3BP (SEQ ID No:20), VPS51 (SEQ ID No:21), VPS52 (SEQ ID No:22) and VPS53 (SEQ ID No:23), more preferably a loss of function mutation in SPTSSA (SEQ ID No:12) and a mutation in a gene selected from the group consisting of C18orf8 and VPS54,
optionally comprising the further step of providing in the individual a lysosomotropic compound, preferably chloroquine, azithromycin, dririthromycin, clarithromycin, erithromycin, promethazine, cyclizine, amodiaquine, telithromycin, tripelennamine, clemastine fumarate, hydroxyzine, rifabutin, mefloquine, chlorcyclyzine, homochlorcyclzine, quinacrine, meclizine, tilorone, bepotastine besilate, vicriviroc, hydroxychloroquine, quinine, arbidol, nelfinavir or tamoxifen, wherein the lysosomotropic compound and itraconazole are provided in synergistically effective amounts.

Embodiment 13: A method for preventing or treating cancer, preferably ovarian cancer, more preferably platinum resistant or refractory epithelilal ovarian cancer in an individual, comprising
administering a therapeutically effective amount of itraconazole to the individual wherein a cancer cell of the individual has a mutation, preferably a loss of function mutation in KIAA1033 (SEQ ID No:13); more preferably a loss of function mutation in KIAA1033 (SEQ ID No:13) and a mutation in a gene selected from the group consisting of NBEAL1 (SEQ ID No:1), C18orf8 (SEQ ID No:2), COG6 (SEQ ID No:3), SLC30A9 (SEQ ID No:4), ARL1 (SEQ ID No:5), UNC50 (SEQ ID No:6), EPT1 (SEQ ID No:7), RTN4IP1 (SEQ ID No:8), SPTLC2 (SEQ ID No:9), SEC14L1 (SEQ ID No:10), CCDC22 (SEQ ID No:11), SPTSSA (SEQ ID No:12), SPTLC1 (SEQ ID No:14), VPS54 (SEQ ID No:15), TSSC1 (SEQ ID No:16), KIF11 (SEQ ID No:17), NDUFS4 (SEQ ID No:18), COMMD4 (SEQ ID No:19), COL4A3BP (SEQ ID No:20), VPS51 (SEQ ID No:21), VPS52 (SEQ ID No:22) and VPS53 (SEQ ID No:23), more preferably a loss of function mutation in KIAA1033 (SEQ ID No:13) and a mutation in a gene selected from the group consisting of C18orf8 and VPS54,
optionally comprising the further step of providing in the individual a lysosomotropic compound, preferably chloroquine, azithromycin, dririthromycin, clarithromycin, erithromycin, promethazine, cyclizine, amodiaquine, telithromycin, tripelennamine, clemastine fumarate, hydroxyzine, rifabutin, mefloquine, chlorcyclyzine, homochlorcyclzine, quinacrine, meclizine, tilorone, bepotastine besilate, vicriviroc, hydroxychloroquine, quinine, arbidol, nelfinavir or tamoxifen, wherein the lysosomotropic compound and itraconazole are provided in synergistically effective amounts.

Embodiment 14: A method for preventing or treating cancer, preferably ovarian cancer, more preferably platinum resistant or refractory epithelilal ovarian cancer in an individual, comprising
administering a therapeutically effective amount of itraconazole to the individual wherein a cancer cell of the individual has a mutation, preferably a loss of function mutation in SPTLC1 (SEQ ID No:14); more preferably a loss of function mutation in SPTLC1 (SEQ ID No:14), and a mutation in a gene selected from the group consisting of NBEAL1 (SEQ ID No:1), C18orf8 (SEQ ID No:2), COG6 (SEQ ID No:3), SLC30A9 (SEQ ID No:4), ARL1 (SEQ ID No:5), UNC50 (SEQ ID No:6), EPT1 (SEQ ID No:7), RTN4IP1 (SEQ ID No:8), SPTLC2 (SEQ ID No:9), SEC14L1 (SEQ ID No:10), CCDC22 (SEQ ID No:11), SPTSSA (SEQ ID No:12), KIAA1033 (SEQ ID No:13), VPS54 (SEQ ID No:15), TSSC1 (SEQ ID No:16), KIF11 (SEQ ID No:17), NDUFS4 (SEQ ID No:18), COMMD4 (SEQ ID No:19), COL4A3BP (SEQ ID No:20), VPS51 (SEQ ID No:21), VPS52 (SEQ ID No:22) and VPS53 (SEQ ID No:23), more preferably a loss of function mutation in SPTLC1 (SEQ ID No:14) and a mutation in a gene selected from the group consisting of C18orf8 and VPS54,
optionally comprising the further step of providing in the individual a lysosomotropic compound, preferably chloroquine, azithromycin, dririthromycin, clarithromycin, erithromycin, promethazine, cyclizine, amodiaquine, telithromycin, tripelennamine, clemastine fumarate, hydroxyzine, rifabutin, mefloquine, chlorcyclyzine, homochlorcyclzine, quinacrine, meclizine, tilorone, bepotastine besilate, vicriviroc, hydroxychloroquine, quinine, arbidol, nelfinavir or tamoxifen, wherein the lysosomotropic compound and itraconazole are provided in synergistically effective amounts.

Embodiment 15: A method for preventing or treating cancer, preferably ovarian cancer, more preferably platinum resistant or refractory epithelilal ovarian cancer in an individual, comprising
administering a therapeutically effective amount of itraconazole to the individual wherein a cancer cell of the individual has a mutation, preferably a loss of function mutation in VPS54 (SEQ ID No:15); more preferably a loss of function mutation in VPS54 (SEQ ID No:15) and a mutation in a gene selected from the group consisting of NBEAL1 (SEQ ID No:1), C18orf8 (SEQ ID No:2), COG6 (SEQ ID No:3), SLC30A9 (SEQ ID No:4), ARL1 (SEQ ID No:5), UNC50 (SEQ ID No:6), EPT1 (SEQ ID No:7), RTN4IP1 (SEQ ID No:8), SPTLC2 (SEQ ID No:9), SEC14L1 (SEQ ID No:10), CCDC22 (SEQ ID No:11), SPTSSA (SEQ ID No:12), KIAA1033 (SEQ ID No:13), SPTLC1 (SEQ ID No:14), TSSC1 (SEQ ID No:16), KIF11 (SEQ ID No:17), NDUFS4 (SEQ ID No:18), COMMD4 (SEQ ID No:19), COL4A3BP (SEQ ID No:20), VPS51 (SEQ ID No:21), VPS52 (SEQ ID No:22) and VPS53 (SEQ ID No:23), more preferably a loss of function mutation in VPS54 (SEQ ID No:15) and a mutation in the C18orf8 gene,
optionally comprising the further step of providing in the individual a lysosomotropic compound, preferably chloroquine, azithromycin, dririthromycin, clarithromycin, erithromycin, promethazine, cyclizine, amodiaquine, telithromycin, tripelennamine, clemastine fumarate, hydroxyzine, rifabutin, mefloquine, chlorcyclyzine, homochlorcyclzine, quinacrine, meclizine, tilorone, bepotastine besilate, vicriviroc, hydroxychloroquine, quinine, arbidol, nelfinavir or tamoxifen, wherein the lysosomotropic compound and itraconazole are provided in synergistically effective amounts.

Embodiment 16: A method for preventing or treating cancer, preferably ovarian cancer, more preferably platinum resistant or refractory epithelilal ovarian cancer in an individual, comprising
administering a therapeutically effective amount of itraconazole to the individual wherein a cancer cell of the individual has a mutation, preferably a loss of function mutation in TSSC1 (SEQ ID No:16); more preferably a loss of function mutation in TSSC1 (SEQ ID No:16) and a mutation in a gene selected from the group consisting of NBEAL1 (SEQ ID No:1), C18orf8 (SEQ ID No:2), COG6 (SEQ ID No:3), SLC30A9 (SEQ ID No:4), ARL1 (SEQ ID No:5), UNC50 (SEQ ID No:6), EPT1 (SEQ ID No:7), RTN4IP1 (SEQ ID No:8), SPTLC2 (SEQ ID No:9), SEC14L1 (SEQ ID No:10), CCDC22 (SEQ ID No:11), SPTSSA (SEQ ID No:12), KIAA1033 (SEQ ID No:13), SPTLC1 (SEQ ID No:14), VPS54 (SEQ ID No:15), KIF11 (SEQ ID No:17), NDUFS4 (SEQ ID No:18), COMMD4 (SEQ ID No:19), COL4A3BP (SEQ ID No:20), VPS51 (SEQ ID No:21), VPS52 (SEQ ID No:22) and VPS53 (SEQ ID No:23), more preferably a loss of function mutation in TSSC1 (SEQ ID No:16) and a mutation in a gene selected from the group consisting of C18orf8 and VPS54,
optionally comprising the further step of providing in the individual a lysosomotropic compound, preferably chloroquine, azithromycin, dririthromycin, clarithromycin, erithromycin, promethazine, cyclizine, amodiaquine, telithromycin, tripelennamine, clemastine fumarate, hydroxyzine, rifabutin, mefloquine, chlorcyclyzine, homochlorcyclzine, quinacrine, meclizine, tilorone, bepotastine besilate, vicriviroc, hydroxychloroquine, quinine, arbidol, nelfinavir or tamoxifen, wherein the lysosomotropic compound and itraconazole are provided in synergistically effective amounts.

Embodiment 17: A method for preventing or treating cancer, preferably ovarian cancer, more preferably platinum resistant or refractory epithelilal ovarian cancer in an individual, comprising
administering a therapeutically effective amount of itraconazole to the individual wherein a cancer cell of the individual has a mutation, preferably a loss of function mutation in KIF11 (SEQ ID No:17); more preferably a loss of function mutation in KIF11 (SEQ ID No:17) and a mutation in a gene selected from the group consisting of NBEAL1 (SEQ ID No:1), C18orf8 (SEQ ID No:2), COG6 (SEQ ID No:3), SLC30A9 (SEQ ID No:4), ARL1 (SEQ ID No:5), UNC50 (SEQ ID No:6), EPT1 (SEQ ID No:7), RTN4IP1 (SEQ ID No:8), SPTLC2 (SEQ ID No:9), SEC14L1 (SEQ ID No:10), CCDC22 (SEQ ID No:11), SPTSSA (SEQ ID No:12), KIAA1033 (SEQ ID No:13), SPTLC1 (SEQ ID No:14), VPS54 (SEQ ID No:15), TSSC1 (SEQ ID No:16), NDUFS4 (SEQ ID No:18), COMMD4 (SEQ ID No:19), COL4A3BP (SEQ ID No:20), VPS51 (SEQ ID No:21), VPS52 (SEQ ID No:22) and VPS53 (SEQ ID No:23), more preferably a loss of function mutation in KIF11 (SEQ ID No:17) and a mutation in a gene selected from the group consisting of C18orf8 and VPS54,
optionally comprising the further step of providing in the individual a lysosomotropic compound, preferably chloroquine, azithromycin, dririthromycin, clarithromycin, erithromycin, promethazine, cyclizine, amodiaquine, telithromycin, tripelennamine, clemastine fumarate, hydroxyzine, rifabutin, mefloquine, chlorcyclyzine, homochlorcyclzine, quinacrine, meclizine, tilorone, bepotastine besilate, vicriviroc, hydroxychloroquine, quinine, arbidol, nelfinavir or tamoxifen, wherein the lysosomotropic compound and itraconazole are provided in synergistically effective amounts.

Embodiment 18: A method for preventing or treating cancer, preferably ovarian cancer, more preferably platinum resistant or refractory epithelilal ovarian cancer in an individual, comprising
administering a therapeutically effective amount of itraconazole to the individual wherein a cancer cell of the individual has a mutation, preferably a loss of function mutation in NDUFS4 (SEQ ID No:18); more preferably a loss of function mutation in NDUFS4 (SEQ ID No:18) and a mutation in a gene selected from the group consisting of NBEAL1 (SEQ ID No:1), C18orf8 (SEQ ID No:2), COG6 (SEQ ID No:3), SLC30A9 (SEQ ID No:4), ARL1 (SEQ ID No:5), UNC50 (SEQ ID No:6), EPT1 (SEQ ID No:7), RTN4IP1 (SEQ ID No:8), SPTLC2 (SEQ ID No:9), SEC14L1 (SEQ ID No:10), CCDC22 (SEQ ID No:11), SPTSSA (SEQ ID No:12), KIAA1033 (SEQ ID No:13), SPTLC1 (SEQ ID No:14), VPS54 (SEQ ID No:15), TSSC1 (SEQ ID No:16), KIF11 (SEQ ID No:17), COMMD4 (SEQ ID No:19), COL4A3BP (SEQ ID No:20), VPS51 (SEQ ID No:21), VPS52 (SEQ ID No:22) and VPS53 (SEQ ID No:23), more preferably a loss of function mutation in NDUFS4 (SEQ ID No:18) and a mutation in a gene selected from the group consisting of C18orf8 and VPS54,
optionally comprising the further step of providing in the individual a lysosomotropic compound, preferably chloroquine, azithromycin, dririthromycin, clarithromycin, erithromycin, promethazine, cyclizine, amodiaquine, telithromycin, tripelennamine, clemastine fumarate, hydroxyzine, rifabutin, mefloquine, chlorcyclyzine, homochlorcyclzine, quinacrine, meclizine, tilorone, bepotastine besilate, vicriviroc, hydroxychloroquine, quinine, arbidol, nelfinavir or tamoxifen, wherein the lysosomotropic compound and itraconazole are provided in synergistically effective amounts.

Embodiment 19: A method for preventing or treating cancer, preferably ovarian cancer, more preferably platinum resistant or refractory epithelilal ovarian cancer in an individual, comprising
administering a therapeutically effective amount of itraconazole to the individual wherein a cancer cell of the individual has a mutation, preferably a loss of function mutation in COMMD4 (SEQ ID No:19); more preferably a loss of function mutation in COMMD4 (SEQ ID No:19) and a mutation in a gene selected from the group consisting of NBEAL1 (SEQ ID No:1), C18orf8 (SEQ ID No:2), COG6 (SEQ ID No:3), SLC30A9 (SEQ ID No:4), ARL1 (SEQ ID No:5), UNC50 (SEQ ID No:6), EPT1 (SEQ ID No:7), RTN4IP1 (SEQ ID No:8), SPTLC2 (SEQ ID No:9), SEC14L1 (SEQ ID No:10), CCDC22 (SEQ ID No:11), SPTSSA (SEQ ID No:12), KIAA1033 (SEQ ID No:13), SPTLC1 (SEQ ID No:14), VPS54 (SEQ ID No:15), TSSC1 (SEQ ID No:16), KIF11 (SEQ ID No:17), NDUFS4 (SEQ ID No:18), COL4A3BP (SEQ ID No:20), VPS51 (SEQ ID No:21), VPS52 (SEQ ID No:22) and VPS53 (SEQ ID No:23), more preferably a loss of function mutation in COMMD4 (SEQ ID No:19) and a mutation in a gene selected from the group consisting of C18orf8 and VPS54,
optionally comprising the further step of providing in the individual a lysosomotropic compound, preferably chloroquine, azithromycin, dririthromycin, clarithromycin, erithromycin, promethazine, cyclizine, amodiaquine, telithromycin, tripelennamine, clemastine fumarate, hydroxyzine, rifabutin, mefloquine, chlorcyclyzine, homochlorcyclzine, quinacrine, meclizine, tilorone, bepotastine besilate, vicriviroc, hydroxychloroquine, quinine, arbidol, nelfinavir or tamoxifen, wherein the lysosomotropic compound and itraconazole are provided in synergistically effective amounts.

Embodiment 20: A method for preventing or treating cancer, preferably ovarian cancer, more preferably platinum resistant or refractory epithelilal ovarian cancer in an individual, comprising
administering a therapeutically effective amount of itraconazole to the individual wherein a cancer cell of the individual has a mutation, preferably a loss of function mutation in COL4A3BP (SEQ ID No:20); more preferably a loss of function mutation in COL4A3BP (SEQ ID No:20) and a mutation in a gene selected from the group consisting of NBEAL1 (SEQ ID No:1), C18orf8 (SEQ ID No:2), COG6 (SEQ ID No:3), SLC30A9 (SEQ ID No:4), ARL1 (SEQ ID No:5), UNC50 (SEQ ID No:6), EPT1 (SEQ ID No:7), RTN4IP1 (SEQ ID No:8), SPTLC2 (SEQ ID No:9), SEC14L1 (SEQ ID No:10), CCDC22 (SEQ ID No:11), SPTSSA (SEQ ID No:12), KIAA1033 (SEQ ID No:13), SPTLC1 (SEQ ID No:14), VPS54 (SEQ ID No:15), TSSC1 (SEQ ID No:16), KIF11 (SEQ ID No:17), NDUFS4 (SEQ ID No:18), COMMD4 (SEQ ID No:19), VPS51 (SEQ ID No:21), VPS52 (SEQ ID No:22) and VPS53 (SEQ ID No:23), more preferably a loss of function mutation in COL4A3BP (SEQ ID No:20) and a mutation in a gene selected from the group consisting of C18orf8 and VPS54,
optionally comprising the further step of providing in the individual a lysosomotropic compound, preferably chloroquine, azithromycin, dririthromycin, clarithromycin, erithromycin, promethazine, cyclizine, amodiaquine, telithromycin, tripelennamine, clemastine fumarate, hydroxyzine, rifabutin, mefloquine, chlorcyclyzine, homochlorcyclzine, quinacrine, meclizine, tilorone, bepotastine besilate, vicriviroc, hydroxychloroquine, quinine, arbidol, nelfinavir or tamoxifen, wherein the lysosomotropic compound and itraconazole are provided in synergistically effective amounts.

Embodiment 21: A method for preventing or treating cancer, preferably ovarian cancer, more preferably platinum resistant or refractory epithelilal ovarian cancer in an individual, comprising
administering a therapeutically effective amount of itraconazole to the individual wherein a cancer cell of the individual has a mutation, preferably a loss of function mutation in VPS51 (SEQ ID No:21); more preferably a loss of function mutation in VPS51 (SEQ ID No:21) and a mutation in a gene selected from the group consisting of NBEAL1 (SEQ ID No:1), C18orf8 (SEQ ID No:2), COG6 (SEQ ID No:3), SLC30A9 (SEQ ID No:4), ARL1 (SEQ ID No:5), UNC50 (SEQ ID No:6), EPT1 (SEQ ID No:7), RTN4IP1 (SEQ ID No:8), SPTLC2 (SEQ ID No:9), SEC14L1 (SEQ ID No:10), CCDC22 (SEQ ID No:11), SPTSSA (SEQ ID No:12), KIAA1033 (SEQ ID No:13), SPTLC1 (SEQ ID No:14), VPS54 (SEQ ID No:15), TSSC1 (SEQ ID No:16), KIF11 (SEQ ID No:17), NDUFS4 (SEQ ID No:18), COMMD4 (SEQ ID No:19), COL4A3BP (SEQ ID No:20), VPS52 (SEQ ID No:22) and VPS53 (SEQ ID No:23), more preferably a loss of function mutation in VPS51 (SEQ ID No:21) and a mutation in a gene selected from the group consisting of C18orf8 and VPS54,
   optionally comprising the further step of providing in the individual a lysosomotropic compound, preferably chloroquine, azithromycin, dririthromycin, clarithromycin, erithromycin, promethazine, cyclizine, amodiaquine, telithromycin, tripelennamine, clemastine fumarate, hydroxyzine, rifabutin, mefloquine, chlorcyclyzine, homochlorcyclzine, quinacrine, meclizine, tilorone, bepotastine besilate, vicriviroc, hydroxychloroquine, quinine, arbidol, nelfinavir or tamoxifen, wherein the lysosomotropic compound and itraconazole are provided in synergistically effective amounts.

Embodiment 22: A method for preventing or treating cancer, preferably ovarian cancer, more preferably platinum resistant or refractory epithelilal ovarian cancer in an individual, comprising
administering a therapeutically effective amount of itraconazole to the individual wherein a cancer cell of the individual has a mutation, preferably a loss of function mutation in VPS52 (SEQ ID No:22); more preferably a loss of function mutation in VPS52 (SEQ ID No:22) and a mutation in a gene selected from the group consisting of NBEAL1 (SEQ ID No:1), C18orf8 (SEQ ID No:2), COG6 (SEQ ID No:3), SLC30A9 (SEQ ID No:4), ARL1 (SEQ ID No:5), UNC50 (SEQ ID No:6), EPT1 (SEQ ID No:7), RTN4IP1 (SEQ ID No:8), SPTLC2 (SEQ ID No:9), SEC14L1 (SEQ ID No:10), CCDC22 (SEQ ID No:11), SPTSSA (SEQ ID No:12), KIAA1033 (SEQ ID No:13), SPTLC1 (SEQ ID No:14), VPS54 (SEQ ID No:15), TSSC1 (SEQ ID No:16), KIF11 (SEQ ID No:17), NDUFS4 (SEQ ID No:18), COMMD4 (SEQ ID No:19), VPS51 (SEQ ID No:21), and VPS53 (SEQ ID No:23), more preferably a loss of function mutation in VPS52 (SEQ ID No:22) and a mutation in a gene selected from the group consisting of C18orf8 and VPS54,
   optionally comprising the further step of providing in the individual a lysosomotropic compound, preferably chloroquine, azithromycin, dririthromycin, clarithromycin, erithromycin, promethazine, cyclizine, amodiaquine, telithromycin, tripelennamine, clemastine fumarate, hydroxyzine, rifabutin, mefloquine, chlorcyclyzine, homochlorcyclzine, quinacrine, meclizine, tilorone, bepotastine besilate, vicriviroc, hydroxychloroquine, quinine, arbidol, nelfinavir or tamoxifen, wherein the lysosomotropic compound and itraconazole are provided in synergistically effective amounts.

Embodiment 23: A method for preventing or treating cancer, preferably ovarian cancer, more preferably platinum resistant or refractory epithelilal ovarian cancer in an individual, comprising
administering a therapeutically effective amount of itraconazole to the individual wherein a cancer cell of the individual has a mutation, preferably a loss of function mutation in VPS53 (SEQ ID No:23); more preferably a loss of function mutation in VPS53 (SEQ ID No:23) and a mutation in a gene selected from the group consisting of NBEAL1 (SEQ ID No:1), C18orf8 (SEQ ID No:2), COG6 (SEQ ID No:3), SLC30A9 (SEQ ID No:4), ARL1 (SEQ ID No:5), UNC50 (SEQ ID No:6), EPT1 (SEQ ID No:7), RTN4IP1 (SEQ ID No:8), SPTLC2 (SEQ ID No:9), SEC14L1 (SEQ ID No:10), CCDC22 (SEQ ID No:11), SPTSSA (SEQ ID No:12), KIAA1033 (SEQ ID No:13), SPTLC1 (SEQ ID No:14), VPS54 (SEQ ID No:15), TSSC1 (SEQ ID No:16), KIF11 (SEQ ID No:17), NDUFS4 (SEQ ID No:18), COMMD4 (SEQ ID No:19), VPS51 (SEQ ID No:21) and VPS52 (SEQ ID No:22), more preferably a loss of function mutation in VPS53 (SEQ ID No:23) and a mutation in a gene selected from the group consisting of C18orf8 and VPS54,
   optionally comprising the further step of providing in the individual a lysosomotropic compound, preferably chloroquine, azithromycin, dririthromycin, clarithromycin, erithromycin, promethazine, cyclizine, amodiaquine, telithromycin, tripelennamine, clemastine fumarate, hydroxyzine, rifabutin, mefloquine, chlorcyclyzine, homochlorcyclzine, quinacrine, meclizine, tilorone, bepotastine besilate, vicriviroc, hydroxychloroquine, quinine, arbidol, nelfinavir or tamoxifen, wherein the lysosomotropic compound and itraconazole are provided in synergistically effective amounts.

The method of any one of the above numerated embodiments may further comprise a step of:
assessing or having assessed a cell of the individual, preferably a cancer cell to determine if the cell has a mutation, preferably a loss of function mutation in a gene selected from the group consisting of: NBEAL1 (SEQ ID No:1), C18orf8 (SEQ ID No:2), COG6 (SEQ ID No:3), SLC30A9 (SEQ ID No:4), ARL1 (SEQ ID No:5), UNC50 (SEQ ID No:6), EPT1 (SEQ ID No:7), RTN4IP1 (SEQ ID No:8), SPTLC2 (SEQ ID No:9), SEC14L1 (SEQ ID No:10), CCDC22 (SEQ ID No:11), SPTSSA (SEQ ID No:12), KIAA1033 (SEQ ID No:13), SPTLC1 (SEQ ID No:14), VPS54 (SEQ ID No:15), TSSC1 (SEQ ID No:16), KIF11 (SEQ ID No:17), NDUFS4 (SEQ ID No:18), COMMD4 (SEQ ID No:19), COL4A3BP (SEQ ID No:20), VPS51 (SEQ ID No:21), VPS52 (SEQ ID No:22) and VPS53 (SEQ ID No:23);
selecting the individual for treatment of cancer with itraconazole where the individual has a mutation, preferably a loss of function mutation in a gene selected from the group consisting of: NBEAL1 (SEQ ID No:1), C18orf8 (SEQ ID No:2), COG6 (SEQ ID No:3), SLC30A9 (SEQ ID No:4), ARL1 (SEQ ID No:5), UNC50 (SEQ ID No:6), EPT1 (SEQ ID No:7), RTN4IP1 (SEQ ID No:8), SPTLC2 (SEQ ID No:9), SEC14L1 (SEQ ID No:10), CCDC22 (SEQ ID No:11), SPTSSA (SEQ ID No:12), KIAA1033 (SEQ ID No:13), SPTLC1 (SEQ ID No:14), VPS54 (SEQ ID No:15), TSSC1 (SEQ ID No:16), KIF11 (SEQ ID No:17), NDUFS4 (SEQ ID No:18), COMMD4 (SEQ ID No:19), COL4A3BP (SEQ ID No:20), VPS51 (SEQ ID No:21), VPS52 (SEQ ID No:22) and VPS53 (SEQ ID No:23).

The method of any one of the above enumerated embodiments may comprise the step of:
determining whether the cell of the individual has reduced production, or no production of a protein or RNA encoded by a gene selected from the group consisting of: NBEAL1 (SEQ ID No:1), C18orf8 (SEQ ID No:2), COG6 (SEQ ID No:3), SLC30A9 (SEQ ID No:4), ARL1 (SEQ ID No:5), UNC50 (SEQ ID No:6), EPT1 (SEQ ID No:7), RTN4IP1 (SEQ ID No:8), SPTLC2 (SEQ ID No:9), SEC14L1 (SEQ ID No:10), CCDC22 (SEQ ID No:11), SPTSSA (SEQ ID No:12), KIAA1033 (SEQ ID No:13), SPTLC1 (SEQ ID No:14), VPS54 (SEQ ID No:15), TSSC1 (SEQ ID No:16), KIF11 (SEQ ID No:17), NDUFS4 (SEQ ID No:18), COMMD4 (SEQ ID No:19), COL4A3BP (SEQ ID No:20), VPS51 (SEQ ID No:21), VPS52 (SEQ ID No:22) and VPS53 (SEQ ID No:23),
   wherein reduced production or no production of a protein or RNA encoded by a gene selected from the group consisting of: NBEAL1 (SEQ ID No:1), C18orf8 (SEQ ID No:2), COG6 (SEQ ID No:3), SLC30A9 (SEQ ID No:4), ARL1 (SEQ ID No:5), UNC50 (SEQ ID No:6), EPT1 (SEQ ID No:7), RTN4IP1 (SEQ ID No:8), SPTLC2 (SEQ ID No:9), SEC14L1 (SEQ ID No:10), CCDC22 (SEQ ID No:11), SPTSSA (SEQ ID No:12), KIAA1033 (SEQ ID No:13), SPTLC1 (SEQ ID No:14), VPS54 (SEQ ID No:15), TSSC1 (SEQ ID No:16), KIF11 (SEQ ID No:17), NDUFS4 (SEQ ID No:18), COMMD4 (SEQ ID No:19), COL4A3BP (SEQ ID No:20), VPS51 (SEQ ID No:21), VPS52 (SEQ ID No:22) and VPS53 (SEQ ID No:23) determines that the cell has a loss of function mutation in the gene,
thereby determining whether the cell has a loss of function mutation in a gene of said group.

### DETAILED DESCRIPTION OF THE INVENTION

Itraconazole is a widely used anti-fungal drug that has been investigated in cancer types including cancer of prostate, lung, ovary, breast, pancreas and biliary tract, and basal cell carcinoma, leukemia and NSCLC, as a monotherapy and in combination with other chemotherapeutic drugs, including daunorubicin, docetaxel, and pemetrexed, the latter combination for example as a chemosensitiser. Response profiles have varied from individual to individual with some individuals demonstrating complete or partial remission and others no response at all.

It has been found herein that cancer cells, especially cancer cells of epithelial origin may contain mutations in specific genes that control an increased sensitivity of those cells to itraconazole either as a monotherapy, or in combination with other chemotherapeutic compounds. In more detail, the CRISPR screen analysis described herein of genes of a panel of itraconazole-sensitive cancer cell lines of epithelial origin identified 20 genes variously associated with in silico predicted pathways including ceramide de novo biosynthesis, Lipid metabolism, Sphingolipid metabolism, Retrograde transport at the Trans-Golgi-Network, Sphingolipid de novo biosynthesis, Intra-Golgi and retrograde Golgi-to-ER traffic, Fabry disease, Gaucher Disease, Globoid Cell Leukodystrophy, Krabbe disease, Metachromatic leukodystrophy, Membrane trafficking, Glycosphingolipid metabolism, Vesicle-mediated transport and Sphingolipid signalling.

Further, CRISPR knockout models that effectively model the loss of function mutations observed in the genes identified in the CRISPR screen analysis also show sensitivity to itraconazole, underscoring an underlying causation or association between the mutations in the genes identified in the CRISPR screen analysis and a sensitivity of cancer cells having these gene mutations to itraconazole.

The inventors have considered the association between the mutations described herein and itraconazole sensitivity in the context of a separate clinical trial in which itraconazole was administered in combination with a lysosomotropic agent. The inventors reasoned that the lysosomotropic agent (hydroxy)chloroquine used in the trial with itraconazole should effectively model at least some of the loss of function gene mutations observed in the CRISPR screen analysis. The clinical trial result supports the findings from the in vitro studies that the gene mutations identified from the CRISPR screen analysis are either causative or associative with an increased itraconazole sensitivity phenotype.

In further in vitro studies of cancer cell lines not having gene mutations identified by the CRISPR screen analysis, it has been observed that those cell lines have increased sensitivity to itraconazole when treated with a lysosomotropic agent such as tamoxifen, or a V-ATPase inhibitor such as Bafilomycin A or Concanamycin. This result is consistent with the finding from the clinical trial described herein.

Without wanting to be bound by hypothesis, taken together, the data presented herein suggest that the gene mutations observed in the CRISPR screen analysis imbue cancer cells having those mutations with sub optimal or no lysosome function. This is observed herein histologically as an induction of lysosomal enlargement, lysosomal dysfunction and lysosomal stress response. The result of sub optimal or no lysosome function may be to increase the sensitivity of those cancer cells having those mutations to itraconazole.

Collectively, the data presented herein strongly indicates that the gene mutations identified in the CRISPR screen analysis can be utilised as basis to select patients for administration with itraconazole, either as a mono therapy, or as a combination therapy for treatment of cancer, and in particular for treatment of a wide range of epithelial cancers or carcinomas.

### 1. DEFINITIONS

For purposes of interpreting this specification, the following definitions will apply and whenever appropriate, terms used in the singular will also include the plural and vice versa.

As used herein, the term “about” in relation to a numerical value x means +/−10%, unless the context dictates otherwise.

“Itraconazole” is a common name for a triazole antifungal compound, the specific chemical structure and IUPAC name of which are well known in the art. It is available commercially (see Merck Index Reg. No. 5262 (12^{th} ed. 1996) and U.S. Pat. No. 4,267,179). As used herein, “itraconazole” includes not only the chemical compound (free base form, also referred to as “free itraconazole”), but also all optical isomers, such as enantiomers, diastereomers, meso compounds, and the like, as well as pharmaceutically acceptable salts, solvates, and prodrugs (such as esters) thereof.

As used herein, ‘loss of function mutation’ and grammatical variants thereof generally refers to a mutation that causes a gene product encoded by, or transcribed from, or translated from the gene to have abnormal quantity and/or abnormal quality. A loss of function mutation may lead to production of an abnormal amount of a gene product, for example an abnormally high, or an abnormally low amount of gene product, or no gene product at all. In one embodiment, the presence of a loss of function mutation may be assessed by determining whether in a subject cancer cell there is reduced or no production of the relevant gene product, such as a protein or RNA. In this embodiment where reduced or no production of the relevant gene product in the subject cancer cell is detected, this determines that the loss of function mutation is present. A loss of function mutation may lead to production of a gene product that has sub-optimal function, or no function, when compared to the function of wild type or normal gene product.

As used herein ‘lysosomotropic compound’ generally refers to a pharmaceutical compound that targets lysosomes or otherwise accumulates in lysosomes, usually as a result of a positive charge on the lysosomotropic compound. Examples of lysosomotropic compounds include V-ATPase inhibitor compounds.

As used herein, ‘synergistically effective amounts’, for example with reference to a combination of compound A and compound B, refers to an amounts of compound A and compound B that provide for a therapeutic effect that is greater than the sum of the effect of compound A when administered alone and compound B when administered alone.

As used herein, the term “pharmaceutically acceptable” means a nontoxic material that does not interfere with the effectiveness of the biological activity of the active ingredient(s).

As used herein, the term “treat”, “treating” or “treatment” in connection to a disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, {e.g., stabilization of a discernible symptom), physiologically, {e.g., stabilization of a physical parameter), or both. The term “alleviating” or “alleviation”, for example in reference to a symptom of a condition, as used herein, refers to reducing at least one of the frequency and amplitude of a symptom of a condition in a patient. In one embodiment, the terms “method for the treatment” or “method for treating”, as used herein, refer to “method to treat”.

As used herein, the term “therapeutically effective amount” refers to an amount of the compound of the invention, e.g. itraconazole; which is sufficient to achieve the stated effect. Accordingly, a therapeutically effective amount of itraconazole; will be an amount sufficient for the treatment or prevention of cancer.

By “therapeutic regimen” is meant the pattern of treatment of an illness, e.g., the pattern of dosing used during the treatment of the disease or disorder.

As used herein, a subject is “in need of” a treatment if such subject would benefit biologically, medically or in quality of life from such treatment.

### 2. DETAILED DESCRIPTION OF THE FIGURES

FIGS. 1A-C. FIG. 1A) Activity area analysis showing the activity of Itraconazole (Itra) in panel of 28 ovarian cancer cell lines. Cells were treated for 5 days with 0-40 μM Itra. FIG. 1B) Left, schematic representation of Itra chemogenetic CRISPR screen. Right, graphs showing DrugZ-calculated normZ score in OVCAR5 and TOV1946 cells. Synergistic/synthetic lethal interactions are reported in red at FDR and p value<0.05. FIG. 1C) Left-middle, Venn diagram showing overlap between the top hits from OVCAR5 and TOV1946 CRISPR screens and table showing common hits. Right, bioinformatic analysis showing pathways involved in the regulation of Itra sensitivity using Patdiph annotated database.

FIGS. 2A-2C. Graphs showing Alamar blue results on cells treated with Itra at a concentration of 0-40 μM for 5 days.

FIGS. 3A-C. FIG. 3A) Western blotting analysis showing Cas9 expression in stable TOV1946 and OVCAR5 cells overexpressing Cas9. B-tubulin and elF4E were used as loading controls. Alamar blue results showing Cas9 activity in Cas9 overexpressing cells infected to express sgRNA targeting LacZ (control condition) or the proteasomal subunits PSMD1 and PSMD2 previously reported to be essential genes. 2 different sgRNA were used for PSMD1 and PSMD2. FIG. 3B) Alalmar blue results showing Itra activity in OVCAR5 and TOV1946 overexpressing Cas9. FIG. 3C) Representative pictures of cells treated in the same conditions as FIG. 3A).

FIGS. 4A-I. FIG. 4A) Sequencing results and alignment of PCR amplicons reporting a homozygous deletion in the exon2 of c18orf gene upstream the AGG PAM sequence (reported in green). H1 represent a c18orf8 knockout cell line. FIG. 4B) Alamar blue results showing increased sensitivity to Itra in c18orf8 knockout cells compared to control. FIG. 4C) Protein translation wild type (top) versus knockout (bottom) c18orf8 resulting from OVCAR5 H1. The bottom protein sequence showed the insertion of a premature stop codon. FIGS. 4D-E) LAMP1 and Rab7 staining of wild type and c18orf8 knockout cells. Higher magnification is reported on the right. FIG. 4F) Western blotting analysis showing knockout of VPS54 in 3 independent clones of OVCAR5. B-tubulin and EIF4E were used as a loading control. FIG. 4G) FILIPIN staining showing cholesterol accumulation in VPS54 knockout cells compared to controls. FIG. 4H) Alamar blue results showing increased sensitivity to Itra in VPS54 knockout cells compared to controls. FIG. 4I) LAMP1 and Rab7 staining of wild type and VPS54 knockout cells. Higher magnification is reported on the right.

FIGS. 5A-B. FIG. 5A) Heat maps showing inhibitory effect of Itra alone (0-40 μM) or in combination with CQ (5-10 μM) in a panel of 28 ovarian cancer cell lines. FIG. 5B) Waterfall plot showing the synergy scores calculated using a Bliss independence model of combinations of Itra and CQ. Synergy score values are ranked from resistant to sensitive cells.

FIGS. 6A-6C. Alamar blue results showing the activity of Itra alone (black, 0-40 μM), or in combination with CQ 5 μM (red) and 10 μM (blue).

FIGS. 7A-7C. Alamar blue results showing the activity CQ 5 μM and 10 μM.

FIGS. 8A-8C. Synergy heat maps obtained using Synergy finder 2.0 software of cells treated in the same conditions as FIG. 5A-B. Itra-CQ synergy scores for each cell line are reported in FIG. 5B.

FIG. 9. Representative pictures (top) and quantification (bottom) of apoptotic rate in OVCAR5 cells treated with different combinations of Itra and CQ.

FIGS. 10A-C. FIG. 10A) and FIG. 10B) Alamar blue results and synergy heat maps of cells treated with Itra alone (black, 0-40 μM), or in combination with CQ 5 μM (red) and 10 μM (blue). FIG. 10C) Graph showing the Itra/CQ synergy score of three independent experiments of control cells (wt and wt #27), c18orf8 KO (#H1) and VPS54 KO (#14,23,24).

FIGS. 11A-B. FIG. 11A) Top, analysis of lysosomal pattern by LAMP1 immunofluorescence and relative quantification in TOV21g treated with Itra 5 μM (15)+/−CQ 5 μM (CQ5). Bottom, Analysis of lysosomal function and relative quantification in TOV21g treated as above. FIG. 11B) Analysis and quantification of TFEB nuclear translocation in TOV1946 stably expressing GFP conjugated TFEB and treated with Itra 5 μM (5) +/−CQ 10 μM (CQ10).

FIGS. 12A-D. FIG. 12A) Swimmer plot showing progression free survival (PFS) in patient enrolled in the HYDRA clinical trial. Different dose levels are reported in green (DL1), Orange (DL2) and blue (DL3). Different types of tumour are labeled with a triangle (low grade serous carcinoma) or a square (High grade serous carcinoma). FIG. 12B) Graph showing change from baseline in tumour size in HYDRA patients. FIGS. 12C-D) Chromatograms showing intra-tumour detection and quantification of Itra (left) and CQ (right) in HYDRA patients pre- and on treatment. Measurements were done using HPLC-MS/MS method. Specific peaks showing retention time for Itra (1.68 minutes) and CQ (0.95 min) are indicated in the m/z graphs reporting analysis in patient HYDRA-005.

FIGS. 13A-B. FIG. 13A) graphs showing immunohistochemistry quantification of cleaved caspase 3 (CC3), p62 and LAMP1 in HYDRA patients slides pre- and on treatment. FIG. 13B) Representative pictures of IHC staining (Hematoxylin-Eosin (HE), CC3, p62 and LAMP1) in patient HYDRA-005 pre and post treatment.

FIG. 14. Tissue heat map obtained using Aperio ImageScope showing treatment induced increased intensity of CC3, p62 and LAMP1 in patient HYDRA-005.

FIGS. 15A-B. FIG. 15A) Phase I rolling six study design. EOC: Epithelial ovarian cancer. DL: Dose-Level. MTD: Maximum tolerated toxicity. DLT: Dose limiting toxicity. RP2D: Recommended phase II dose. FIG. 15B) Quantification of Ki-67 staining in HYDRA patients pre- and on treatment (graphs on the left) and representative picture (right) of patient HYDRA-005.

FIGS. 16A-B: Summary of all adverse events and treatment related adverse events.

FIG. 17: DrugZ analysis showing ranking of GARP subunits VPS51, VPS52, VPS53 and VPS54 in TOV1946 and OVCAR5.

### 3. MODES OF CARRYING OUT THE INVENTION

The various embodiments described below will be understood to be applicable to carrying out the invention of the methods and enumerated embodiments described above.

As described herein, the invention relates to methods for assessing individuals to determine whether they have, or are at risk of cancer that is sensitive to itraconazole therapy whether given as a monotherapy or combination therapy, and for treating said individuals with itraconazole.

3.1 Biomarkers for Selection/Identification of Individuals Having Itraconazole-Sensitive Cancer

The invention utilises biomarkers in the form of mutations, especially loss of function mutations of genes listed in Table 1 for the purpose assessing individuals to determine whether they have, or are at risk of having cancer that is sensitive to itraconazole therapy whether given as a monotherapy or combination therapy, and for treating said individuals with itraconazole.

Table 1 refers to a genomic sequence for each biomarker. Each of the genomic sequences referred to in Table 1 defines a biomarker having a normal function.

In embodiments of the invention, a cancer cell that contains all of the genomic sequences in Table 1 has a lower likelihood of sensitivity to itraconazole treatment.

In embodiments of the invention, a cancer cell that contains a mutant form of one or more genomic sequences in Table 1, in particular a mutant form comprising a loss of function mutation, and therefore that contains fewer than all of the genomic sequences in Table 1, has a higher likelihood of sensitivity to itraconazole treatment. Such a cancer cell may have reduced production of a protein or RNA encoded by a genomic sequence in Table 1.

TABLE 1

| | GENE | | SEQ ID | | | |
|---|---|---|---|---|---|---|
| Gene | ID | Accession no. | No. | GO - Biological process | Pathway/function | ALIASES |
| NBEAL1 | 65065 | NC_000002.12: | 1 | protein localization | Control of SREBP2 | A530083I02Rik, |
| | | 203013784- | | | processing and cholesterol | ALS2CR16, |
| | | 203225194 | | | metabolism | ALS2CR17 |
| C18orf8 | 29919 | NC_000018.10: | 2 | autophagy, regulation of | Componement of the CCZ1- | RMC1; MIC1; |
| | | 23503470- | | autophagy | MON1 RAB7A guanine | Mic-1; |
| | | 23531822 | | | exchange factor (GEF). Acts | WDR98; |
| | | | | | as a positive regulator of | HsT2591 |
| | | | | | CCZ1-MON1A/B function | |
| | | | | | necessary for | |
| | | | | | endosomal/autophagic flux | |
| | | | | | and efficient RAB7A | |
| | | | | | localization | |
| COG6 | 57511 | NC_000013.11: | 3 | endoplasmic reticulum | This gene encodes a subunit | COD2; SHNS; |
| | | 39655627- | | to Golgi vesicle- | of the conserved oligomeric | CDG2L |
| | | 39791666 | | mediated transport, | Golgi complex that is required | |
| | | | | glycosylation, intra-Golgi | for maintaining normal | |
| | | | | vesicle-mediated | structure and activity of the | |
| | | | | transport, protein | Golgi apparatus. The encoded | |
| | | | | transport | protein is organized with | |
| | | | | | conserved oligomeric Golgi | |
| | | | | | complex components 5, 7 and | |
| | | | | | 8 into a sub-complex referred | |
| | | | | | to as lobe B. Alternative | |
| | | | | | splicing results in multiple | |
| | | | | | transcript variants. [provided | |
| | | | | | by RefSeq, February 2009] | |
| SLC30A9 | 10463 | NC_ 000004.12: | 4 | Ion transport, | Acts as a zinc transporter | HUEL; ZNT9; |
| | | 41990502- | | Transcription, | involved in intracellular zinc | GAC63; |
| | | 42090461 | | Transcription regulation, | homeostasis | C4orf1; |
| | | | | Transport, Zinc transport | (PubMed: 28334855). | BILAPES |
| | | | | | Functions as a secondary | |
| | | | | | coactivator for nuclear | |
| | | | | | receptors by cooperating with | |
| | | | | | p160 coactivators subtypes. | |
| | | | | | Plays a role in transcriptional | |
| | | | | | activation of Wnt-responsive | |
| | | | | | genes (By similarity). | |
| ARL1 | 400 | NC_000012.12: | 5 | activation of | GTP-binding protein that | ARFL1 |
| | | c101407820- | | phospholipase D | recruits several effectors, such | |
| | | 101393116 | | activity, Golgi | as golgins, arfaptins and Arf- | |
| | | | | organization, | GEFs to the trans-Golgi | |
| | | | | intracellular protein | network, and modulates their | |
| | | | | transport, protein | functions at the Golgi complex | |
| | | | | localization to Golgi | (PubMed: 9624189, | |
| | | | | apparatus, retrograde | PubMed: 21239483, | |
| | | | | transport, endosome to | PubMed: 27436755, | |
| | | | | Golgi, toxin metabolic | PubMed: 22679020, | |
| | | | | process, vesicle- | PubMed: 27373159). Plays | |
| | | | | mediated transport | thereby a role in a wide range | |
| | | | | | of fundamental cellular | |
| | | | | | processes, including cell | |
| | | | | | polarity, innate immunity, or | |
| | | | | | protein secretion mediated by | |
| | | | | | arfaptins, which were shown to | |
| | | | | | play a role in maintaining | |
| | | | | | insulin secretion from | |
| | | | | | pancreatic beta cells | |
| | | | | | (PubMed: 22981988). | |
| UNC50 | 25972 | NC_000002.12: | 6 | Protein transport, | May be involved in cell surface | URP; GMH1; |
| | | 98608589- | | Transport | expression of neuronal | UNCL; |
| | | 98618515 | | | nicotinic receptors. Binds RNA | HSD23; |
| | | | | | (By similarity). | PDLs22 |
| EPT1 | 85465 | NC_000002.12: | 7 | phosphatidylethanolamine | Ethanolaminephosphotransferase | SELENOI, |
| | | 26346143- | | biosynthetic process, | that catalyzes the transfer | SELI; SEPI; |
| | | 26395885 | | Lipid biosynthesis, Lipid | of phosphoethanolamine/PE | SPG81 |
| | | | | metabolism, | from CDP-ethanolamine to | |
| | | | | Phospholipid | lipid acceptors, the final step in | |
| | | | | biosynthesis, | the synthesis of PE via the | |
| | | | | Phospholipid | ‘Kennedy’ pathway | |
| | | | | metabolism | (PubMed: 17132865, | |
| | | | | | PubMed: 28052917, | |
| | | | | | PubMed: 29500230). PE is the | |
| | | | | | second most abundant | |
| | | | | | phospholipid of membranes in | |
| | | | | | mammals and is involved in | |
| | | | | | various membrane-related | |
| | | | | | cellular processes | |
| | | | | | (PubMed: 17132865). The | |
| | | | | | enzyme is critical for the | |
| | | | | | synthesis of several PE | |
| | | | | | species and could also | |
| | | | | | catalyze the synthesis of | |
| | | | | | ether-linked phospholipids like | |
| | | | | | plasmanyl- and plasmenyl-PE | |
| | | | | | which could explain it is | |
| | | | | | required for proper myelination | |
| | | | | | and neurodevelopment | |
| | | | | | (PubMed: 29500230). | |
| RTN4IP1 | 84816 | NC_000006.12: | 8 | nervous system | Plays a role in the regulation of | NIMP; OPA10 |
| | | c106630921- | | development, regulation | retinal ganglion cell (RGC) | |
| | | 106559237 | | of dendrite development | neurite outgrowth, and hence | |
| | | | | | in the development of the inner | |
| | | | | | retina and optic nerve. | |
| | | | | | Appears to be a potent | |
| | | | | | inhibitor of regeneration | |
| | | | | | following spinal cord injury. | |
| SPTLC2 | 9517 | NC_000014.9: | 9 | Lipid metabolism, | Serine palmitoyltransferase | LCB2; SPT2; |
| | | c77616663- | | Sphingolipid metabolism | (SPT). The heterodimer | HSN1C; |
| | | 77505997 | | | formed with LCB1/SPTLC1 | LCB2A; |
| | | | | | constitutes the catalytic core. | NSAN1C; |
| | | | | | The composition of the serine | hLCB2a |
| | | | | | palmitoyltransferase (SPT) | |
| | | | | | complex determines the | |
| | | | | | substrate preference. The | |
| | | | | | SPTLC1-SPTLC2-SPTSSA | |
| | | | | | complex shows a strong | |
| | | | | | preference for C16-CoA | |
| | | | | | substrate, while the SPTLC1- | |
| | | | | | SPTLC2-SPTSSB complex | |
| | | | | | displays a preference for C18- | |
| | | | | | CoA substrate. Plays an | |
| | | | | | important role in de novo | |
| | | | | | sphyngolipid biosynthesis | |
| | | | | | which is crucial for | |
| | | | | | adipogenesis (By similarity). | |
| SEC14L1 | 6397 | NC_000017.11: | 10 | Immunity, Innate | May play a role in innate | SEC14L; |
| | | 77088685- | | immunity | immunity by inhibiting the | PRELID4A |
| | | 77217101 | | | antiviral RIG-I signaling | |
| | | | | | pathway. In this pathway, | |
| | | | | | functions as a negative | |
| | | | | | regulator of DDX58/RIG-I, the | |
| | | | | | cytoplasmic sensor of viral | |
| | | | | | nucleic acids. Prevents the | |
| | | | | | interaction of DDX58 with | |
| | | | | | MAVS/IPS1, an important step | |
| | | | | | in signal propagation | |
| | | | | | (PubMed: 23843640). May | |
| | | | | | also regulate the SLC18A3 | |
| | | | | | and SLC5A7 cholinergic | |
| | | | | | transporters | |
| | | | | | (PubMed: 17092608). | |
| CCDC22 | 28952 | NC_000023.11: | 11 | cellular copper ion | Involved in regulation of NF- | JM1; RTSC2; |
| | | 49235470- | | homeostasis, | kappa-B signaling. Promotes | CXorf37 |
| | | 49250526 | | cytoplasmic | ubiquitination of I-kappa-B- | |
| | | | | sequestering of NF- | kinase subunit IKBKB and its | |
| | | | | kappaB, endocytic | subsequent proteasomal | |
| | | | | recycling, Golgi to | degradation leading to NF- | |
| | | | | plasma membrane | kappa-B activation; the | |
| | | | | transport, negative | function may involve | |
| | | | | regulation of I-kappaB | association with COMMD8 | |
| | | | | kinase/NF-kappaB | and a CUL1-dependent E3 | |
| | | | | signaling, positive | ubiquitin ligase complex. May | |
| | | | | regulation of I-kappaB | down-regulate NF-kappa-B | |
| | | | | kinase/NF-kappaB | activity via association with | |
| | | | | signaling, positive | COMMD1 and involving a | |
| | | | | regulation of ubiquitin- | CUL2-dependent E3 ubiquitin | |
| | | | | dependent protein | ligase complex. Regulates the | |
| | | | | catabolic process, post- | cellular localization of COMM | |
| | | | | translational protein | domain-containing proteins, | |
| | | | | modification, protein | such as COMMD1 and | |
| | | | | transport, protein | COMMD10 | |
| | | | | ubiquitination, | (PubMed: 23563313). | |
| | | | | retrograde transport, | Component of the CCC | |
| | | | | endosome to plasma | complex, which is involved in | |
| | | | | membrane | the regulation of endosomal | |
| | | | | | recycling of surface proteins, | |
| | | | | | including integrins, signaling | |
| | | | | | receptor and channels. The | |
| | | | | | CCC complex associates with | |
| | | | | | SNX17, retriever and WASH | |
| | | | | | complexes to prevent | |
| | | | | | lysosomal degradation and | |
| | | | | | promote cell surface recycling | |
| | | | | | of numerous cargos such as | |
| | | | | | integrins ITGA5: ITGB1 | |
| | | | | | (PubMed: 28892079, | |
| | | | | | PubMed: 25355947). Plays a | |
| | | | | | role in copper ion | |
| | | | | | homeostasis. Involved in | |
| | | | | | copper-dependent ATP7A | |
| | | | | | trafficking between the trans- | |
| | | | | | Golgi network and vesicles in | |
| | | | | | the cell periphery; the function | |
| | | | | | is proposed to depend on its | |
| | | | | | association within the CCC | |
| | | | | | complex and cooperation with | |
| | | | | | the WASH complex on early | |
| | | | | | endosomes | |
| | | | | | (PubMed: 25355947). | |
| SPTSSA | 171546 | NC_000014.9: | 12 | Lipid metabolism, | Stimulates the activity of | SSSPTA; |
| | | c34462240- | | Sphingolipid metabolism | serine palmitoyltransferase | C14orf147 |
| | | 34432788 | | | (SPT). The composition of the | |
| | | | | | serine palmitoyltransferase | |
| | | | | | (SPT) complex determines the | |
| | | | | | substrate preference. The | |
| | | | | | SPTLC1-SPTLC2-SPTSSA | |
| | | | | | complex shows a strong | |
| | | | | | preference for C16-CoA | |
| | | | | | substrate, while the SPTLC1- | |
| | | | | | SPTLC3-SPTSSA isozyme | |
| | | | | | uses both C14-CoA and C16- | |
| | | | | | CoA as substrates, with a | |
| | | | | | slight preference for C14-CoA | |
| | | | | | (PubMed: 19416851). Plays a | |
| | | | | | role in MBOAT7 location to | |
| | | | | | mitochondria-associated | |
| | | | | | membranes (MAMs), may me | |
| | | | | | involved in fatty acid | |
| | | | | | remodeling | |
| | | | | | phosphatidylinositol (PI) | |
| | | | | | (PubMed: 23510452). | |
| KIAA1033 | 23325 | NC_000012.12: | 13 | endosomal transport, | Acts as a component of the | WASHC4; |
| | | 105107726- | | endosome organization, | WASH core complex that | SWIP; MRT43 |
| | | 105169134 | | protein transport | functions as a nucleation- | |
| | | | | | promoting factor (NPF) at the | |
| | | | | | surface of endosomes, where | |
| | | | | | it recruits and activates the | |
| | | | | | Arp2/3 complex to induce actin | |
| | | | | | polymerization, playing a key | |
| | | | | | role in the fission of tubules | |
| | | | | | that serve as transport | |
| | | | | | intermediates during | |
| | | | | | endosome sorting. | |
| SPTLC1 | 10558 | NC_000009.12: | 14 | Lipid metabolism, | Serine palmitoyltransferase | HSN1; LBC1; |
| | | c92115413- | | Sphingolipid metabolism | (SPT) (PubMed: 19416851). | LCB1; SPT1; |
| | | 92031141 | | | The heterodimer formed with | SPTI; HSAN1 |
| | | | | | SPTLC2 or SPTLC3 | |
| | | | | | constitutes the catalytic core | |
| | | | | | (PubMed: 19416851). The | |
| | | | | | composition of the serine | |
| | | | | | palmitoyltransferase (SPT) | |
| | | | | | complex determines the | |
| | | | | | substrate preference | |
| | | | | | (PubMed: 19416851). The | |
| | | | | | SPTLC1-SPTLC2-SPTSSA | |
| | | | | | complex shows a strong | |
| | | | | | preference for C16-CoA | |
| | | | | | substrate, while the SPTLC1- | |
| | | | | | SPTLC3-SPTSSA isozyme | |
| | | | | | uses both C14-CoA and C16- | |
| | | | | | CoA as substrates, with a | |
| | | | | | slight preference for C14-CoA | |
| | | | | | (PubMed: 19416851). The | |
| | | | | | SPTLC1-SPTLC2-SPTSSB | |
| | | | | | complex shows a strong | |
| | | | | | preference for C18-CoA | |
| | | | | | substrate, while the SPTLC1- | |
| | | | | | SPTLC3-SPTSSB isozyme | |
| | | | | | displays an ability to use a | |
| | | | | | broader range of acyl-CoAs, | |
| | | | | | without apparent preference | |
| | | | | | (PubMed: 19416851). | |
| | | | | | Required for adipocyte cell | |
| | | | | | viability and metabolic | |
| | | | | | homeostasis (By similarity) | |
| VPS54 | 51542 | NC 000002.12: | 15 | Golgi to vacuole | cts as component of the GARP | WR; HCC8; |
| | | c64019428- | | transport, homeostasis | complex that is involved in | SLP-8p; |
| | | 63892149 | | of number of cells within | retrograde transport from early | VPS54L; |
| | | | | a tissue, lysosomal | and late endosomes to the | hVps54L; |
| | | | | transport, | trans-Golgi network (TGN). | PPP1R164 |
| | | | | musculoskeletal | The GARP complex is | |
| | | | | movement, | required for the maintenance | |
| | | | | neurofilament | of the cycling of mannose 6- | |
| | | | | cytoskeleton | phosphate receptors between | |
| | | | | organization, protein | the TGN and endosomes, this | |
| | | | | transport, regulation of | cycling is necessary for proper | |
| | | | | growth, retrograde | lysosomal sorting of acid | |
| | | | | transport, endosome to | hydrolases such as CTSD | |
| | | | | Golgi | (PubMed: 18367545). Within | |
| | | | | | the GARP complex, required | |
| | | | | | to tether the complex to the | |
| | | | | | TGN. Not involved in | |
| | | | | | endocytic recycling | |
| | | | | | (PubMed: 25799061). | |
| TSSC1 | 7260 | NC_000002.12: | 16 | endocytic recycling, | Acts as a component of | EIPR1; |
| | | c3377818- | | positive regulation of | endosomal retrieval | EIPR-1 |
| | | 3188968 | | endocytic recycling, | machinery that is involved in | |
| | | | | positive regulation of | protein transport from early | |
| | | | | retrograde transport, | endosomes to either recycling | |
| | | | | endosome to Golgi, | endosomes or the trans-Golgi | |
| | | | | protein ubiquitination, | network (PubMed: 27440922). | |
| | | | | regulation of insulin | Mediates the recruitment of | |
| | | | | secretion | Golgi-associated retrograde | |
| | | | | | protein (GARP) complex to the | |
| | | | | | trans-Golgi network and | |
| | | | | | controls early endosome-to- | |
| | | | | | Golgi transport of internalized | |
| | | | | | protein(PubMed: 27440922). | |
| | | | | | Promotes the recycling of | |
| | | | | | internalized transferrin | |
| | | | | | receptor (TFRC) to the plasma | |
| | | | | | membrane through interaction | |
| | | | | | with endosome-associated | |
| | | | | | recycling protein (EARP) | |
| | | | | | complex (PubMed: 27440922). | |
| | | | | | Controls proper insulin | |
| | | | | | distribution and secretion, and | |
| | | | | | retention of cargo in mature | |
| | | | | | dense core vesicles (By | |
| | | | | | similarity). Required for the | |
| | | | | | stability of the endosome- | |
| | | | | | associated retrograde protein | |
| | | | | | (EARP) complex subunits and | |
| | | | | | for proper localization and | |
| | | | | | association of EARP with | |
| | | | | | membranes (By similarity). | |
| KIF11 | 3832 | NC_000010.11: | 17 | antigen processing and | Motor protein required for | EG5; HKSP; |
| | | 92593130- | | presentation of | establishing a bipolar spindle | KNSL1; |
| | | 92655395 | | exogenous peptide | during mitosis | MCLMR; |
| | | | | antigen via MHC class II, | (PubMed: 19001501). | TRIP5 |
| | | | | cell division, | Required in non-mitotic cells | |
| | | | | microtubule-based | for transport of secretory | |
| | | | | movement, mitotic cell | proteins from the Golgi | |
| | | | | cycle, mitotic | complex to the cell surface | |
| | | | | centrosome separation, | (PubMed: 23857769). | |
| | | | | mitotic spindle | | |
| | | | | assembly, mitotic | | |
| | | | | spindle organization, | | |
| | | | | regulation of mitotic | | |
| | | | | centrosome separation, | | |
| | | | | retrograde vesicle- | | |
| | | | | mediated transport, | | |
| | | | | Golgi to endoplasmic | | |
| | | | | reticulum, spindle | | |
| | | | | organization | | |
| NDUFS4 | 4724 | NC_000005.10: | 18 | Electron transport, | Accessory subunit of the | AQDQ; CI-18; |
| | | 53560610- | | Respiratory chain, | mitochondrial membrane | MC1DN1; CI- |
| | | 53683338 | | Transport | respiratory chain NADH | AQDQ; CI-18 |
| | | | | | dehydrogenase (Complex I), | kDa |
| | | | | | that is believed not to be | |
| | | | | | involved in catalysis. Complex | |
| | | | | | I functions in the transfer of | |
| | | | | | electrons from NADH to the | |
| | | | | | respiratory chain. The | |
| | | | | | immediate electron acceptor | |
| | | | | | for the enzyme is believed to | |
| | | | | | be ubiquinone. | |
| COMMD4 | 54939 | NC_000015.10: | 19 | Transcription, | May modulate activity of cullin- | |
| | | 75336035- | | Transcription | RING E3 ubiquitin ligase | |
| | | 75343227 | | regulation, Ubl | (CRL) complexes | |
| | | | | conjugation pathway | (PubMed: 21778237). Down- | |
| | | | | | regulates activation of NF- | |
| | | | | | kappa-B. | |
| COL4A3BP | 10087 | NC_000005.10: | 20 | Lipid transport, | Shelters ceramides and | CERT1; |
| | | c75511981- | | Transport | diacylglycerol lipids inside its | CERT; GPBP; |
| | | 75368486 | | | START domain and mediates | CERTL; |
| | | | | | the intracellular trafficking of | MRD34; |
| | | | | | ceramides and diacylglycerol | STARD11 |
| | | | | | lipids in a non-vesicular | |
| | | | | | manner. | |
| VPS51 | 738 | NC_000011.10: | 21 | autophagy, brain | Acts as component of the | FFR; ANG2; |
| | | 65096214- | | morphogenesis, | GARP complex that is | ANG3; |
| | | 65111862 | | endocytic recycling, | involved in retrograde | PCH13; |
| | | | | Golgi organization, | transport from early and late | C11orf2; |
| | | | | Golgi vesicle transport, | endosomes to the trans-Golgi | C11orf3 |
| | | | | lipid transport, | network (TGN). The GARP | |
| | | | | lysosomal transport, | complex is required for the | |
| | | | | protein transport, | maintenance of protein | |
| | | | | retrograde transport, | retrieval from endosomes to | |
| | | | | endosome to Golgi | the TGN, acid hydrolase | |
| | | | | | sorting, lysosome function, | |
| | | | | | endosomal cholesterol traffic | |
| | | | | | and autophagy. VPS51 | |
| | | | | | participates in retrograde | |
| | | | | | transport of acid hydrolase | |
| | | | | | receptors, likely by promoting | |
| | | | | | tethering and SNARE- | |
| | | | | | dependent fusion of | |
| | | | | | endosome-derived carriers to | |
| | | | | | the TGN | |
| | | | | | (PubMed: 20685960). Acts as | |
| | | | | | component of the EARP | |
| | | | | | complex that is involved in | |
| | | | | | endocytic recycling. The | |
| | | | | | EARP complex associates | |
| | | | | | with Rab4-positive | |
| | | | | | endosomes and promotes | |
| | | | | | recycling of internalized | |
| | | | | | transferrin receptor (TFRC) to | |
| | | | | | the plasma membrane | |
| | | | | | (PubMed: 25799061). | |
| VPS52 | 6293 | NC_000006.12: | 22 | ectodermal cell | Acts as component of the | ARE1; SAC2; |
| | | c33271965- | | differentiation, | GARP complex that is | SACM2L; |
| | | 33250272 | | embryonic ectodermal | involved in retrograde | dJ1033B10.5 |
| | | | | digestive tract | transport from early and late | |
| | | | | development, endocytic | endosomes to the trans-Golgi | |
| | | | | recycling, Golgi to | network (TGN). The GARP | |
| | | | | vacuole transport, | complex is required for the | |
| | | | | lysosomal transport, | maintenance of the cycling of | |
| | | | | protein transport, | mannose 6-phosphate | |
| | | | | retrograde transport, | receptors between the TGN | |
| | | | | endosome to Golgi | and endosomes, this cycling | |
| | | | | | is necessary for proper | |
| | | | | | lysosomal sorting of acid | |
| | | | | | hydrolases such as CTSD | |
| | | | | | (PubMed: 15878329, | |
| | | | | | PubMed: 18367545). Acts as | |
| | | | | | component of the EARP | |
| | | | | | complex that is involved in | |
| | | | | | endocytic recycling. The | |
| | | | | | EARP complex associates | |
| | | | | | with Rab4-positive | |
| | | | | | endosomes and promotes | |
| | | | | | recycling of internalized | |
| | | | | | transferrin receptor (TFRC) to | |
| | | | | | the plasma membrane | |
| | | | | | (PubMed: 25799061). | |
| VPS53 | 55275 | NC_000017.11: | 23 | endocytic recycling, | Acts as component of the | HCCS1; |
| | | c714846-508668 | | lysosomal transport, | GARP complex that is | PCH2E; |
| | | | | protein transport, | involved in retrograde | hVps53L; |
| | | | | retrograde transport, | transport from early and late | pp13624 |
| | | | | endosome to Golgi | endosomes to the trans-Golgi | |
| | | | | | network (TGN). The GARP | |
| | | | | | complex is required for the | |
| | | | | | maintenance of the cycling of | |
| | | | | | mannose 6-phosphate | |
| | | | | | receptors between the TGN | |
| | | | | | and endosomes, this cycling | |
| | | | | | is necessary for proper | |
| | | | | | lysosomal sorting of acid | |
| | | | | | hydrolases such as CTSD | |
| | | | | | (PubMed: 15878329, | |
| | | | | | PubMed: 18367545). Acts as | |
| | | | | | component of the EARP | |
| | | | | | complex that is involved in | |
| | | | | | endocytic recycling. The | |
| | | | | | EARP complex associates | |
| | | | | | with Rab4-positive | |
| | | | | | endosomes and promotes | |
| | | | | | recycling of internalized | |
| | | | | | transferrin receptor (TFRC) to | |
| | | | | | the plasma membrane | |
| | | | | | (PubMed: 25799061). | |

In one embodiment the loss of function mutation is a deletion of all or part of an exon or intron or regulatory region of a gene listed in Table 1.

In one embodiment the loss of function mutation is an insertion of one or more nucleotides in an exon or intron or regulatory region of a gene listed in Table 1.

In one embodiment the loss of function mutation is a substitution of one or more nucleotides in an exon or intron or regulatory region of a gene listed in Table 1.

In one embodiment the loss of function mutation is a deletion event of chromosome comprising a gene listed in Table 1.

In one embodiment the loss of function mutation is a duplication event of a chromosome comprising a gene listed in Table 1.

In one embodiment the loss of function mutation is a inversion event of a chromosome comprising a gene listed in Table 1.

In one embodiment the loss of function mutation is an insertion event of a chromosome comprising a gene listed in Table 1.

In one embodiment the loss of function mutation is a translocation event of a chromosome comprising a gene listed in Table 1.

In one embodiment the loss of function mutation is a somatic mutation.

In one embodiment the loss of function mutation causes a frame shift mutation in a gene listed in Table 1.

In one embodiment the loss of function mutation creates a stop signal in a gene listed in Table 1.

In one embodiment, the loss of function mutation results in no production, or in reduced production of a gene product in the form of a protein or RNA encoded by a gene listed in Table 1.

In one embodiment the loss of function mutation leads to the production of a gene product, preferably a protein that has sub-optimal function, or no function, when compared to the function of a gene product encoded by a gene listed in Table 1.

3.1.1 NBEAL1

In one embodiment, the mutation in NBEAL1 causes, or is associated with a defect in LDLR-mediated cholesterol uptake by the cancer cell as regulated by sterol regulatory element binding protein 2 (SREBP2), especially by minimising or precluding an interaction between NBEAL1 and sterol regulatory element binding protein cleavage activating protein (SOAP), or by minimising or precluding an interaction between NBEAL1 and adipoQ receptor family member 3 protein (PAQR).

In this embodiment the loss of function mutation may be a mutation of the NBEAL1 gene that leads to a reduction in production of a normal NBEAL1 protein, or no production of normal NBEAL1 protein at all.

A reduction in production of normal NBEAL1 protein in a cancer cell is likely to be associated with minimised interaction between NBEAL1 and SOAP, or NBEAL1 and PAQR, thereby leading to sub-optimal regulation of SREBP2 activity in mediating LDLR in cholesterol uptake. An absence of production of normal NBEAL1 protein in a cancer cell is likely to preclude interaction between NBEAL1 and SOAP, or NBEAL1 and PAQR thereby leaving SREBP2 activity unregulated by SOAP or PAQR.

A reduction or absence of production of normal NBEAL1 protein may be associated with production of NBEAL1 protein that is unable to functionally interact with SOAP or PAQR. Such an NBEAL1 protein may lack one or more functional domains required for SOAP or PAQR interaction. These NBEAL1 protein mutants may be detected by standard techniques.

A loss of function mutation may present as an absence of all or part of a genomic sequence for NBEAL as shown in Table 1, or an epigenetic change in a regulatory region of NBEAL1 gene that leads to decreased NBEAL1 gene expression. These mutation events may be detected by standard techniques as described further herein.

3.1.2 C18orf8

In one embodiment the mutation in C18orf8 causes, or is associated with a defect in the lysosomal maturation, thereby inducing lysosomal enlargement and dysfunction and inhibiting autophagy in a cancer cell. The mutation may minimise or preclude an interaction between C18orf8 and components of the CCZ1-Mon1 complex. The CCZ1-Mon1 complex is required for recruitment of Rab7 at the LE/L lysosomes and for lysosomal maturation. The mutation in C18orf8 may minimise or preclude the interaction between these components and C18orf8 thereby preventing the formation of a functional CCZ1-Mon1 complex.

In this embodiment the loss of function mutation may be a mutation of the C18orf8 gene that leads to a reduction in production of a normal C18orf8 protein, or no production of normal C18orf8 protein at all.

A reduction in production of normal C18orf8 protein in a cancer cell is likely to be associated with minimised interaction between C18orf8 and components of the CCZ1-Mon1 complex, thereby leading to sub-optimal recruitment of Rab7 protein at LE/L lysosomes. An absence of production of normal C18orf8 protein in a cancer cell is likely to preclude interaction between C18orf8 and components of CCZ1-Mon1 thereby leaving sub-optimal or no CCZ-Mon1 function in the cancer cell.

A reduction or absence of production of normal C18orf8 protein may be associated with production of C18orf8 protein that is unable to functionally interact with components of CCZ1-Mon1. Such a C18orf8 protein may lack one or more functional domains required for CCZ1-Mon1 assembly. These C18orf8 protein mutants may be detected by standard techniques.

A loss of function mutation may present as an absence of all or part of a genomic sequence for C18orf8 as shown in Table 1, or an epigenetic change in a regulatory region of C18orf8 gene that leads to decreased C18orf8 gene expression. These mutation events may be detected by standard techniques as described further herein.

3.1.3 COG6

In one embodiment the mutation in COG6 causes, or is associated with a defect in the ER to Golgi vehicle-mediated transport or intra-Golgi vehicle-mediated transport in the cancer cell especially by minimising or precluding an interaction between COG6 and Golgi complex components 5, 7, and 8, thereby preventing the formation of a functional lobe B subcomplex.

In this embodiment the loss of function mutation may be a mutation of the COG6 gene that leads to a reduction in production of a normal COG6 protein, or no production of normal COG6 protein at all.

A reduction in production of normal COG6 protein in a cancer cell is likely to be associated with minimised interaction between COG6 and the components of the functional lobe B subcomplex, thereby leading to sub-optimal intra Golgi and ER to Golgi transport of protein chains. An absence of production of normal COG6 protein in a cancer cell is likely to preclude interaction between COG6 and Golgi complex components 5, 7 and 8 thereby impairing transport from ER to Golgi and within the Golgi.

A reduction or absence of production of normal COG6 protein may be associated with production of COG6 protein that is unable to functionally interact with Golgi complex components 5, 7 and 8. Such an COG6 protein may lack one or more functional domains required for Golgi complex components 5, 7 and 8 interaction. These COG6 protein mutants may be detected by standard techniques.

A loss of function mutation may present as an absence of all or part of a genomic sequence for COG6 as shown in Table 1, or an epigenetic change in a regulatory region of COG6 gene that leads to decreased COG6 gene expression. These mutation events may be detected by standard techniques as described further herein.

3.1.4 SLC30A9

A loss of function mutation may present as an absence of all or part of a genomic sequence for SLC30A9 as shown in Table 1, or an epigenetic change in a regulatory region of SLC30A9 gene that leads to decreased SLC30A9 gene expression. These mutation events may be detected by standard techniques as described further herein

3.1.5 ARL1

A loss of function mutation may present as an absence of all or part of a genomic sequence for ARL1 as shown in Table 1, or an epigenetic change in a regulatory region of ARL1 gene that leads to decreased ARL1 gene expression. These mutation events may be detected by standard techniques as described further herein.

3.1.6 UNC50

A loss of function mutation may present as an absence of all or part of a genomic sequence for UNC50 as shown in Table 1, or an epigenetic change in a regulatory region of UNC50 gene that leads to decreased UNC50 gene expression. These mutation events may be detected by standard techniques as described further herein.

3.1.7 EPT1

In one embodiment the mutation in EPT1 causes, or is associated with a defect in phosphatidylethanolamine (PE) synthesis in a cancer cell by minimising or precluding the catalysis of transfer of PE to lipid acceptors.

In this embodiment the loss of function mutation may be a mutation of the EPT1 gene that leads to a reduction in production of a normal EPT1 protein, or no production of normal EPT1 protein at all.

A reduction in production of normal EPT1 protein in a cancer cell is likely to be associated with minimised transfer of PE to lipid acceptors, thereby leading to sub-optimal PE synthesis. An absence of production of normal EPT1 protein in a cancer cell is likely to require the cancer cell to source PE from other cellular compartments, thereby stressing the cancer cell.

A reduction or absence of production of normal EPT1 protein may be associated with production of EPT1 protein that is unable to catalyse the transfer of PE to lipid acceptor molecules. Such an EPT1 protein may lack one or more catalytic domains controlling PE transfer. These EPT1 protein mutants may be detected by standard techniques.

A loss of function mutation may present as an absence of all or part of a genomic sequence for EPT1 as shown in Table 1, or an epigenetic change in a regulatory region of EPT1 gene that leads to decreased EPT1 gene expression. These mutation events may be detected by standard techniques as described further herein.

3.1.8 RTN4IP1

A loss of function mutation may present as an absence of all or part of a genomic sequence for RTN4IP1 as shown in Table 1, or an epigenetic change in a regulatory region of RTN4IP1 gene that leads to decreased RTN4IP1 gene expression. These mutation events may be detected by standard techniques as described further herein.

3.1.9 SPTLC2

In one embodiment the mutation in SPTLC2 causes, or is associated with a defect in the lipid or sphinolipid pathway of the cancer cell as regulated by serine palmitoyltransferase activity arising from heterodimers containing SPTLC2, especially SPTLC1/SPLTC2.

The mutation may minimise or preclude an interaction between SPTLC1 and SPTLC2, or SPTLC2 and SPTSSA, or SPTLC2 and SPTSSB.

In this embodiment the loss of function mutation may be a mutation of the SPTLC2 gene that leads to a reduction in production of a normal SPTLC2 protein, or no production of normal SPTLC2 protein at all.

A reduction in production of normal SPTLC2 protein in a cancer cell is likely to be associated with minimised interaction between SPTLC2 and any one of SPTLC1, SPTSSA and SPTSSB, thereby leading to sub-optimal serine palmitoyltransferase activity. An absence of production of normal SPTLC2 protein in a cancer cell is likely to preclude interaction between between SPTLC2 and any one of SPTLC1, SPTSSA and SPTSSB thereby ostensibly ablating serine palmitoyltransferase activity in a cancer cell.

A reduction or absence of production of normal SPTLC2 protein may be associated with production of SPTLC2 protein that is unable to functionally interact with any one of SPTLC1, SPTSSA and SPTSSB. Such an SPTLC2 protein may lack one or more functional domains required for these interactions. These SPTLC2 protein mutants may be detected by standard techniques.

A loss of function mutation may present as an absence of all or part of a genomic sequence for SPTLC2 as shown in Table 1, or an epigenetic change in a regulatory region of SPTLC2 gene that leads to decreased SPTLC2 gene expression. These mutation events may be detected by standard techniques as described further herein.

3.1.10 SEC14L1

A loss of function mutation may present as an absence of all or part of a genomic sequence for SEC14L1 as shown in Table 1, or an epigenetic change in a regulatory region of SEC14L1 gene that leads to decreased SEC14L1 gene expression. These mutation events may be detected by standard techniques as described further herein.

3.1.11 CCDC22

In one embodiment the mutation in CCDC22 causes, or is associated with a defect in prevention of lysosomal degradation of cell surface proteins of the cancer cell as mediated by SNX17 and WASH complexes when CCDC22 is associated with CCD93 and COMMD proteins in a CCC complex.

The mutation in CCDC22 may minimise or preclude an interaction between CCDC22 and CCD93, or CCDC22 and COMMD proteins.

In this embodiment the loss of function mutation may be a mutation of the CCDC22 gene that leads to a reduction in production of a normal CCDC22 protein, or no production of normal CCDC22 protein at all.

A reduction in production of normal CCDC22 protein in a cancer cell is likely to be associated with minimised interaction between CCDC22 and CCD93, or CCDC22 and COMMD proteins, thereby leading to sub-optimal regulation of prevention of lysosomal degradation of cell surface proteins. An absence of production of normal CCDC22 protein in a cancer cell is likely to preclude interaction between CCDC22 and CCD93, or CCDC22 and COMMD proteins thereby leaving certain cell surface proteins of the cancer cell exposed to lysosomal degradation.

A reduction or absence of production of normal CCDC22 protein may be associated with production of CCDC22 protein that is unable to functionally interact with CCD93, or COMMD proteins. Such an CCDC22 protein may lack one or more functional domains required for CCD93 or COMMD proteins interaction for producing a functional CCC complex. These CCDC22 protein mutants may be detected by standard techniques.

A loss of function mutation may present as an absence of all or part of a genomic sequence for CCDC22 as shown in Table 1, or an epigenetic change in a regulatory region of CCDC22 gene that leads to decreased CCDC22 gene expression. These mutation events may be detected by standard techniques as described further herein.

3.1.12 SPTSSA

In one embodiment the mutation in SPTSSA causes, or is associated with a defect in the lipid or sphinolipid pathway of the cancer cell as regulated by serine palmitoyltransferase activity arising from heterotrimers containing SPTSSA, especially SPTLC1/SPLTC2/SPTSSA and SPTLC1/SPTLC3/SPTSSA.

The mutation may minimise or preclude an interaction between SPTSSA and SPTLC1, or SPTSSA and SPTLC2, or SPTSSA and SPTLC3.

In this embodiment the loss of function mutation may be a mutation of the SPTSSA gene that leads to a reduction in production of a normal SPTSSA protein, or no production of normal SPTSSA protein at all.

A reduction in production of normal SPTSSA protein in a cancer cell is likely to be associated with minimised interaction between SPTSSA and any one of SPTLC1, SPTLC2 and SPTLC3, thereby leading to sub-optimal serine palmitoyltransferase activity. An absence of production of normal SPTSSA protein in a cancer cell is likely to preclude interaction between between SPTSSA and any one of SPTLCC1, SPTLC2, or SPTLC3, thereby ostensibly ablating serine palmitoyltransferase activity in a cancer cell.

A reduction or absence of production of normal SPTSSA protein may be associated with production of SPTSSA protein that is unable to functionally interact with any one of SPTLC1, SPTLC2, or SPTLC3. Such an SPTSSA protein may lack one or more functional domains required for these interactions. These SPTSSA protein mutants may be detected by standard techniques.

A loss of function mutation may present as an absence of all or part of a genomic sequence for SPTSSA as shown in Table 1, or an epigenetic change in a regulatory region of SPTSSA gene that leads to decreased SPTSSA gene expression. These mutation events may be detected by standard techniques as described further herein.

3.1.13 KIAA1033

A loss of function mutation may present as an absence of all or part of a genomic sequence for KIAA1033 as shown in Table 1, or an epigenetic change in a regulatory region of KIAA1033 gene that leads to decreased KIAA1033 gene expression. These mutation events may be detected by standard techniques as described further herein.

3.1.14 SPTLC1

In one embodiment the mutation in SPTLC1 causes, or is associated with a defect in the lipid or sphinolipid pathway of the cancer cell as regulated by serine palmitoyltransferase activity arising from heterodimers containing SPTLC1, especially SPTLC1/SPLTC2 and SPTLC1/SPTLC3.

The mutation may minimise or preclude an interaction between SPTLC1 and SPTLC2, or SPTLC1 and SPTLC3, or SPTLC1 and SPTSSA, or SPTLC1 and SPTSSB.

In this embodiment the loss of function mutation may be a mutation of the SPTLC1 gene that leads to a reduction in production of a normal SPTLC1 protein, or no production of normal SPTLC1 protein at all.

A reduction in production of normal SPTLC1 protein in a cancer cell is likely to be associated with minimised interaction between SPTLC1 and any one of SPTLC2, SPTLC3, SPTSSA and SPTSSB, thereby leading to sub-optimal serine palmitoyltransferase activity. An absence of production of normal SPTLC1 protein in a cancer cell is likely to preclude interaction between between SPTLC1 and any one of SPTLC2, SPTLC3, SPTSSA and SPTSSB thereby ostensibly ablating serine palmitoyltransferase activity in a cancer cell.

A reduction or absence of production of normal SPTLC1 protein may be associated with production of SPTLC1 protein that is unable to functionally interact with any one of SPTLC2, SPTLC3, SPTSSA and SPTSSB. Such an SPTLC1 protein may lack one or more functional domains required for these interactions. These SPTLC1 protein mutants may be detected by standard techniques.

A loss of function mutation may present as an absence of all or part of a genomic sequence for SPTLC1 as shown in Table 1, or an epigenetic change in a regulatory region of SPTLC1 gene that leads to decreased SPTLC1 gene expression. These mutation events may be detected by standard techniques as described further herein.

3.1.15 VPS54

In one embodiment the mutation in VPS54 causes, or is associated with a defect in retrograde transport from late endosomes to the trans Golgi network, or with recycling of mannose 6 phosphate receptor (M6PR) in the cancer cell, either as mediated by the Golgi associated retrograde protein (GARP) complex which comprises VPS54.

The mutation may minimise or preclude an interaction between VPS54 and components of the GARP complex. The GARP complex regulates retrograde transport from late endosomes to the trans Golgi network. A functional GARP complex comprises the following components: ANG2, VPS52, VPS53, and VPS54. The mutation in VPS54 may minimise or preclude the interaction between these components and VPS54 thereby preventing the formation of a functional GARP complex.

In this embodiment the loss of function mutation may be a mutation of the VPS54 gene that leads to a reduction in production of a normal VPS54 protein, or no production of normal VPS54 protein at all.

A reduction in production of normal VPS54 protein in a cancer cell is likely to be associated with minimised interaction between VPS54 and components of the GARP complex, thereby leading to impaired retrograde transport and M6PR recycling. An absence of production of normal VPS54 protein in a cancer cell is likely to preclude interaction between VPS54 and components of GARP thereby leaving sub-optimal or no GARP function in the cancer cell.

A reduction or absence of production of normal VPS54 protein may be associated with production of VPS54 protein that is unable to functionally interact with components of GARP. Such a VPS54 protein may lack one or more functional domains required for GARP assembly. These VPS54 protein mutants may be detected by standard techniques.

A loss of function mutation may present as an absence of all or part of a genomic sequence for VPS54 as shown in Table 1, or an epigenetic change in a regulatory region of VPS54 gene that leads to decreased VPS54 gene expression. These mutation events may be detected by standard techniques as described further herein.

3.1.16 TSSC1

In one embodiment the mutation in TSSC1 causes, or is associated with a defect in the early endosome retrieval pathway of the cancer cell, especially by minimising or precluding an interaction between TSSC1 and Golgi associated retrograde protein (GARP) complexes (composed of ANG2, VPS52, VPS53, and VPS54 subunits) or by minimising or precluding an interaction between TSSC1 and endosome associated retrograde protein (EARP) complexes (composed of ANG2, VPS52, VPS53, and Syndetin subunits).

In this embodiment the loss of function mutation may be a mutation of the TSSC1 gene that leads to a reduction in production of a normal TSSC1 protein, or no production of normal TSSC1 protein at all.

A reduction in production of normal TSSC1 protein in a cancer cell is likely to be associated with minimised interaction between TSSC1 and GARP complexes, or TSSC1 and EARP complexes, thereby leading to sub-optimal endocytic recycling or sub optimal retrograde transport at the Golgi. An absence of production of normal TSSC1 protein in a cancer cell is likely to preclude interaction between TSSC1 and GARP or EARP complexes thereby interfering with endocytic protein sorting within the cancer cell.

A reduction or absence of production of normal TSSC1 protein may be associated with production of TSSC1 protein that is unable to functionally interact with GARP or EARP complexes. Such an TSSC1 protein may lack one or more functional domains required for GARP or EARP complex interaction. These TSSC1 protein mutants may be detected by standard techniques.

A loss of function mutation may present as an absence of all or part of a genomic sequence for TSSC1 as shown in Table 1, or an epigenetic change in a regulatory region of TSSC1 gene that leads to decreased TSSC1 gene expression. These mutation events may be detected by standard techniques as described further herein.

3.1.17 KIF11

In one embodiment the mutation in KIF1 1 causes, or is associated with a defect in the transport of secretory proteins from the trans Golgi network to the periphery of the cancer cell, especially by minimising or precluding the cross linking or sliding of anti parallel microtubules emanating from the trans Golgi network.

In this embodiment the loss of function mutation may be a mutation of the KIF11 gene that leads to a reduction in production of a normal KIF11 protein, or no production of normal KIF11 protein at all.

A reduction in production of normal KIF11 protein in a cancer cell is likely to be associated with minimised cross linking or sliding of trans Golgi network associated microtubules, leading to sub-optimal transport of secretory proteins to the cancer cell surface. An absence of production of normal KIF11 protein in a cancer cell is likely to preclude interaction between KIF11 and anti parallel microtubules emanating from the trans Golgi network.

A reduction or absence of production of normal KIF11 protein may be associated with production of KIF11 protein that is unable to cross link or slide anti parallel microtubules associated with the trans Golgi network. Such an KIF11 protein may lack one or more functional domains required for this interaction. These KIF11 protein mutants may be detected by standard techniques.

A loss of function mutation may present as an absence of all or part of a genomic sequence for KIF11 as shown in Table 1, or an epigenetic change in a regulatory region of KIF11 gene that leads to decreased KIF11 gene expression. These mutation events may be detected by standard techniques as described further herein.

3.1.18 NDUFS4

A loss of function mutation may present as an absence of all or part of a genomic sequence for NDUFS4 as shown in Table 1, or an epigenetic change in a regulatory region of NDUFS4 gene that leads to decreased NDUFS4 gene expression. These mutation events may be detected by standard techniques as described further herein.

3.1.19 COMMD4

A loss of function mutation may present as an absence of all or part of a genomic sequence for COMMD4 as shown in Table 1, or an epigenetic change in a regulatory region of COMMD4 gene that leads to decreased COMMD4 gene expression. These mutation events may be detected by standard techniques as described further herein.

3.1.20 COL4A3BP

In one embodiment the mutation in COL4A3BP causes, or is associated with a defect in intra-cellular lipid transport in the cancer cell, especially transport of ceramides and diacylglycerol lipids, especially by minimising or precluding an interaction between steroidogenic acute regulatory protein related lipid-transfer (START) domain of COLA3BP and these lipids.

In this embodiment the loss of function mutation may be a mutation of the COL4A3BP gene that leads to a reduction in production of a normal COL4A3BP protein, especially COL4A3BP having a functional START domain, or no production of normal COL4A3BP protein at all.

A reduction in production of normal COL4A3BP protein in a cancer cell is likely to be associated with minimised interaction between the START domain and ceramides and diacylglycerol lipids, thereby leading to sub-optimal intracellular trafficking of these lipids within cancer cells, potentially effecting the amount of lipid available for lysosome development. An absence of production of normal COL4A3BP protein in a cancer cell is likely to preclude interaction between the START domain and these lipids thereby reducing or ablating intracellular lipid trafficking.

A reduction or absence of production of normal COL4A3BP protein may be associated with production of COL4A3BP protein that does not have a functional START domain. These COL4A3BP protein mutants may be detected by standard techniques.

A loss of function mutation may present as an absence of all or part of a genomic sequence for COL4A3BP as shown in Table 1, or an epigenetic change in a regulatory region of COL4A3BP gene that leads to decreased COL4A3BP gene expression. These mutation events may be detected by standard techniques as described further herein.

3.1.21 VPS51

A loss of function mutation may present as an absence of all or part of a genomic sequence for VPS51 as shown in Table 1, or an epigenetic change in a regulatory region of VPS51 gene that leads to decreased VPS51 gene expression. These mutation events may be detected by standard techniques as described further herein.

3.1.22 VPS52

A loss of function mutation may present as an absence of all or part of a genomic sequence for VPS52 as shown in Table 1, or an epigenetic change in a regulatory region of VPS52 gene that leads to decreased VPS52 gene expression. These mutation events may be detected by standard techniques as described further herein.

3.1.23 VPS53

A loss of function mutation may present as an absence of all or part of a genomic sequence for VPS53 as shown in Table 1, or an epigenetic change in a regulatory region of VPS53 gene that leads to decreased VPS53 gene expression. These mutation events may be detected by standard techniques as described further herein.

3.2 Individuals

An individual to whom the methods of the invention are applied is mammalian, preferably a human being.

An individual may not have cancer at the time of treatment. Such an individual may be at risk for cancer i.e. may have one or more risk factors for cancer. The individual may have had prior surgery or chemotherapy for removal and/or treatment of cancer. The individual may be in remission. The invention may be applied to such an individual to prevent the development of cancer, or to prevent cancer.

In another embodiment, an individual may have cancer at the time of treatment. The cancer may be benign or malignant. The cancer may be a primary or metastatic cancer. The cancer may be a solid tumour or encapsulated tumour. The cancer may be disseminated. Such an individual may be asymptomatic for cancer, or symptomatic for cancer. The invention may be applied to such an individual to treat or ameliorate or alleviate cancer.

In one embodiment, the individual to be administered itraconazole has a cancer, preferably a carcinoma, i.e. an epithelial cancer. Examples of epithelial cancer include cancers of prostate, lung, ovarian, breast, pancreatic, renal and biliary tract, and basal cell carcinoma.

In one embodiment, the individual to be administered itraconazole has a refractory or platinum resistant ovarian cancer.

In one embodiment, the individual to be administered itraconazole has a cancer of non epithelial origin, such as a leukemia, sarcoma or neuronal tumors.

3.3 Screening Individuals for Itraconazole Sensitivity

Individuals may be screened for itraconazole sensitivity generally by assessing whether cells of the individual have a biomarker in the form of a mutation of a gene as described in Table 1, or a loss of production of a gene product encoded by a gene listed in Table 1.

In a particularly preferred embodiment, an individual may be selected for treatment or prevention of cancer, or screened for sensitivity to itraconazole therapy, or assessed for likelihood of having cells that are sensitive to itraconazole therapy by assessing whether cancer cells of the cancer in need of treatment in the individual have a mutation of a gene listed in Table 1.

In another embodiment, an individual may be selected for treatment or prevention of cancer, or screened for sensitivity to itraconazole therapy, or assessed for likelihood of having cells that are sensitive to itraconazole therapy by assessing whether non cancer cells in the individual have a mutation of a gene listed in Table 1. The detection of non cancer cells having the relevant mutation may indirectly indicate the presence of cancer cells having the same or similar mutation and hence sensitivity to itraconazole.

In another embodiment, an individual may be selected for treatment or prevention of cancer, or screened for sensitivity to itraconazole therapy, or assessed for likelihood of having cells that are sensitive to itraconazole therapy by assessing whether a body fluid, such as blood or plasma contains a gene product indicating that the individual has a mutation of a gene listed in Table 1. Nucleic acids in circulating blood may be detected by standard amplification techniques including PCR. Proteins may be detected by standard serological techniques.

Whole cancer cells, or fragments or lysates thereof may be obtained from an individual in the form of a solid biopsy or cytological smear by routine techniques.

The identity of cells as cancer cells may be determined by routine serological, histological or molecular techniques known in the art for detection of a relevant cancer cell.

In one embodiment, the method may involve the step of obtaining, or being provided with cancer cells from the individual in need of treatment for said cancer, thereby forming a test sample.

The method may comprise further steps of comparing the test sample with a control defining the amount or activity or function of a gene product of a gene having a sequence described by an accession number listed in Table 1, thereby utilising the control sample to determine whether the test sample comprises a gene product of a gene listed in Table 1 that is abnormal in quantity, preferably reduced or absent, or abnormal in function.

In one embodiment, a control may be may be derived from a single individual. In another embodiment, a control may be derived from a cohort of individuals.

A control may provide a reference point against which a determination regarding implementation of subsequent prophylaxis or therapy with itraconazole can be made. The determination may be made on the basis of the comparison between test sample and the control.

In certain embodiments, the control may be provided in the form of data that has been derived by another party, and/or prior to assessment of the subject for treatment. For example, the control may be derived from a commercial database or a publically available database.

In one embodiment the individual is selected for treatment or prevention of cancer, or screened for sensitivity of cancer cells to itraconazole, or administered itraconazole for treatment of cancer where a cancer cell of the individual comprises a gene product that is abnormal with respect to amount or function as compared with a control.

In certain embodiments, the samples to be tested are body fluids such as blood, serum, plasma, urine, tears, saliva, CSF and the like.

In other embodiments where the cancer is a solid tumor, the sample may be a solid tissue core as obtainable from a needle biopsy.

In certain embodiments, the sample from the individual may require processing prior to assessment of a loss of function mutation. For example, the sample may be centrifuged or diluted to a particular concentration or adjusted to a particular pH prior to testing. Conversely, it may be desirable to concentrate a sample that is too dilute, prior to testing. A sample may be subjected to routine histological, serological or molecular biological process.

In certain embodiments detection means may be utilised that bind directly to the gene product or relevant portion thereof to detect for the presence and/or quantity of the gene product or relevant portion thereof. In other embodiments a probe that binds with the detection means may be measured to indirectly detect for the presence and/or quantity of the gene product or relevant portion thereof. Such a probe or detection means may be an antibody or fragment thereof or a nucleic acid, such as an oligomer.

In one embodiment the individual is selected for treatment or prevention of cancer, or screened for sensitivity of cancer cells to itraconazole, or administered itraconazole for treatment of cancer where a cancer cell of the individual has a reduced production or no production of a protein or RNA encoded by a gene selected from the group consisting of: NBEAL1 (SEQ ID No:1), C18orf8 (SEQ ID No:2), COG6 (SEQ ID No:3), SLC30A9 (SEQ ID No:4), ARL1 (SEQ ID No:5), UNC50 (SEQ ID No:6), EPT1 (SEQ ID No:7), RTN4IP1 (SEQ ID No:8), SPTLC2 (SEQ ID No:9), SEC14L1 (SEQ ID No:10), CCDC22 (SEQ ID No:11), SPTSSA (SEQ ID No:12), KIAA1033 (SEQ ID No:13), SPTLC1 (SEQ ID No:14), VPS54 (SEQ ID No:15), TSSC1 (SEQ ID No:16), KIF11 (SEQ ID No:17), NDUFS4 (SEQ ID No:18), COMMD4 (SEQ ID No:19), COL4A3BP (SEQ ID No:20), VPS51 (SEQ ID No:21), VPS52 (SEQ ID No:22) and VPS53 (SEQ ID No:23). The amount of such a protein or RNA can be directly detected using standard techniques including serological techniques, or amplification techniques and the detected amount can be compared with a control as described above to determine whether the individual has a reduced production of the protein. Further, the amount of such a protein or RNA can be indirectly detected by determining whether the cell contains a gene mutation that results in decreased production or no production of the protein. Decreased production can be detected by analysing the nucleotide sequence of the gene in the cell to determine whether the gene contains a mutation such as a deletion, substitution or insertion that would lead to decreased or no production of a gene product of a gene listed in Table 1. Further, decreased production can be detected by determining the methylation status of the gene in the cell to determine whether there is an epigenetic change in the gene that would lead to decreased or no production of a gene product of a gene listed in Table 1.

3.4 Pharmaceutical Compositions and Administration

Itraconazole and the pharmaceutically acceptable salts can be used as therapeutically active substances, e.g. in the form of pharmaceutical preparations. The pharmaceutical preparations can be administered orally, e.g. in the form of tablets, coated tablets, dragées, hard and soft gelatin capsules, solutions, emulsions or suspensions. The administration can, however, also be effected rectally, e.g. in the form of suppositories, or parenterally, e.g. in the form of injection solutions.

Itraconazole and the pharmaceutically acceptable salts thereof can be processed with pharmaceutically inert, inorganic or organic carriers for the production of pharmaceutical preparations. Lactose, corn starch or derivatives thereof, talc, stearic acids or its salts and the like can be used, for example, as such carriers for tablets, coated tablets, dragees and hard gelatin capsules. Suitable carriers for soft gelatin capsules are, for example, vegetable oils, waxes, fats, semi-solid and liquid polyols and the like. Depending on the nature of the active substance no carriers are however usually required in the case of soft gelatin capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical preparations can, moreover, contain pharmaceutically acceptable auxiliary substances such as preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

Medicaments containing itraconazole and the pharmaceutically acceptable salts and a therapeutically inert carrier are also provided by the present invention, as is a process for their production, which comprises itraconazole and the pharmaceutically acceptable salts and, if desired, one or more other therapeutically valuable substances into a galenical administration form together with one or more therapeutically inert carriers.

The dosage can vary within wide limits and will, of course, have to be adjusted to the individual requirements in each particular case. In the case of oral administration the dosage for adults can vary from about 0.01 mg to about 1000 mg per day of itraconazole and the pharmaceutically acceptable salts. The daily dosage may be administered as single dose or in divided doses and, in addition, the upper limit can also be exceeded when this is found to be indicated.

In one embodiment itraconazole is administered in an amount from 100 to 600 mg daily for a period of 4 weeks to 9 months.

The pharmaceutical preparations may conveniently contain about 1-500 mg, particularly 1-100 mg, of itraconazole.

### EXAMPLES

Matherials and Methods

Preclinical Studies

Cell Lines and Cell Culture

Ovarian cancer lines were obtained from different sources and cultured in either OSE (Wisent), RPMI 1640 supplemented with 10 mM HEPES (Life Technologies), DMEM (Life Technologies), or DMEM/F12 (Wisent), with 10% fetal bovine serum (FBS, Gibco) as previously reported³³. 293T cells were obtained from ATCC. All cell lines were cultured at 37° C. in a 5% CO2 humidified incubator. All cell lines were routinely tested to confirm the absence of Mycoplasma using the Mycoalert Detection Kit (Lonza).

Antibodies, Drugs and Reagents

Anti-Cas9 (#sc-517386) antibody was purchased from Santa Cruz Biotechnology. Anti-beta Tubulin (#ab6046) antibody, anti-p62 (ab56416) were purchased from Abcam. Anti-elF4E (#610270) and antibodies were purchased from BD BiosciencesAnti-LAMP1 (#9091) and anti-Rab7 (#9367) and anti-CC3 (#9661) were purchased from Cell Signaling Technology. Anti-VPS54 antibody (13327-1-AP) was purchased from Proteintech. Anti-Ki-67 (clone: MIB1, M7240) was purchased from Agilent Dako.

Secondary antibodies used for Western blotting were purchased from Licor. Secondary antibodies used from immunofluorescence, Hoechst (NucBlue® Live Ready Probes) and DAPI were purchased from Thermo Fisher Scientific.

Itra and CQ were purchased from Sigma-Aldrich. Alamar blue cell viability reagent was purchased from Thermo Fisher Scientific. Puromycin and blasticydin solutions were purchased from Invivogen.

Filipin III from Streptomyces filipinensis reagent was purchased from Sigma-Aldrich. Lysosomal Intracellular Activity Assay Kit was purchased from Biovision. Polybrene Infection/Transfection reagent was purchased from Milllipore. The QIAamp Blood Maxi Kit used for genomic isolation of CRISPR samples was purchased from QIAGEN.

Genomic DNA isolation kit was purchased from Norgen Biotech Corp. High fidelity master mix used for PCR was purchased from New England Biolabs. All-Prep DNA/RNA/miRNA universal Co-Isolation kit was purchased from QIAGEN. UltraView Detection Kit was purchased from Vantana.

Lentiviral Constructs, Lentivirus Generation and Infection

Lenti-Cas9-2A-Blast (Plasmid #73310) was purchased from Addgene. Lentivirus containing control LacZ and sgRNA targeting the essential genes PSMB1 and PSMD2 coding for proteosomal subunits were kindly provided by the Princess Margaret Genomic Centre (Toronto, ON, Canada). Lentiviral particles were generated by co-transfection of 293T cells with packaging plasmids psPAX2 (Addgene) and pMD2.G (Addgene) together with Lenti-Cas9-2A-Blast using Lipofectamine 3000 according to the manufacturer's instructions. Virus supernatant was harvested 48 and 72 hrs post-transfection. Cell lines were transduced with lentiviral supernatant in the presence of 8 μg/ml polybrene. Infected cells were selected for 48 hrs in puromycin containing media (OVCAR5 3.5 μg/ml and TOV1946 2 μg/ml) or 7 days in 5 μg/ml blasticidin containing media. Lentiviral particles used to knock out VPS54 and c18orf8 were purchased from Horizon discovery (Edit-R Human All-in-one Lentiviral VPS54 sgRNA, #VSGH11936-247734196 and #VSGH11936-247699202 respectively) and cells were infected according to the manufacturer's instructions.

Drug Treatments and Alamar Blue Assay

Cell viability was determined using the Alamar blue assay according to the manufacturer's protocol. Briefly, cells were plated in 96-well plates (2-4×10³cells/100 μL/well) and let attach overnight. Then they were treated with serial dilutions of Itra at a final concentration of 0-40 μM with or without chloroquine 5-10 μM. Cells were tested after 5 days adding Alamar blue solution to each well, incubating for 6 hours and measuring absorbance with a microplate reader (FLUOstar Omega, BMG Labtech) at a test wavelength of 550 nm. All the experiments were performed independently 3 times.

CRISPR Screen

Production of the whole genome sgRNA library TKOV3 lentivirus, 293T cells were transfected with psPAX2 (lentiviral packaging; Addgene #12260), pMD2.G (VSV-G envelope; Addgene #12259), and TKOV3 (Toronto KnockOut CRISPR Library; Addgene #90294) as previously reported³⁴.

For transduction of OVCAR5 AND TOV1946 cells, the TKOV3 virus was added with 8 μg/ml Polybrene in 15 cm dishes. Cells were selected with puromycin at 48 h post-infection. After selection cells were let grown for 5 days to stabilize and then divided in triplicates. Then Itra was added to separate replicates at a final concentration of 1 μM, with one set of replicates receiving no drug treatment. Both drug-treated and untreated replicates were not allowed to reach confluence in the 15 cm dishes. Cells were lifted, counted, and re-plated at the coverage stated above, and the excess cell pellets were frozen at −20° C. as a time point. Once at least 8 doublings were reached from T0, the screens were terminated, and pellets frozen at −20° C. Coverage of screens was kept at 400 cells per gRNA.

Genomic DNA (gDNA) was isolated from the frozen cell pellets using the QIAamp Blood Maxi Kit (Qiagen), submitted for PCR and next generation sequencing and analyzed as previously reported³⁴.

DNA Sequencing of c18orf8

Genomic DNA from CRISPR and control cells was isolated from one million cells pellets using the Genomic DNA Isolation Kit from Norgen Biotech Corp. DNA purity and concentration was determined using Nanodrop (Thermo Fisher Scientific). Then, a region in Exon2 (targeted by c18orf8 sgRNA) was amplified using the Q5 High-Fidelity 2X Master Mix (New England Biolabs) and the following primers: forward primer CTTGCTGCTTTTCCCTCTCA, reverse primer ACCTAAATGAGATGGGATTCCT. C18orf8 specific PCR band was isolated from agarose gel using the QIAquick Gel Extraction kit (QIAGEN) and eluted in water. Samples were submitted for sequencing using the above primers to the ACGT Corp (//acgtcorp.com/) (Toronto,ON, Canada).

Western Blotting

Proteins were extracted from cell lines by RIPA buffer (NORGEN Biotek Corp) with protease and phosphatase inhibitors (Roche). After centrifugation at 10,000 g supernatants were boiled in Laemmli buffer for 10 min and proteins were resolved by SDS-PAGE in Bolt 4-12% Bis-Tris Plus Gels (Thermo Fisher Scientific). Proteins were subsequently transferred onto PVDF membrane (Fisher scientific) and blocked for 1 hour with Odyssey Blocking Buffer in TBS (Licor), incubated overnight with primary antibodies Anti-beta Tubulin (#ab6046), anti-elF4E (#610270) anti-VPS54 antibody (13327-1-AP), and for 1 hour at room temperature in IRDye 680RD and IRDye 800CW conjugated IgG (Licor). Western blots were visualized using the Odyssey Infrared Imaging System (LI-COR Biosciences). Anti-beta Tubulin (#ab6046) and anti-elF4E (#610270) were used as loading control.

FILIPIN Staining

20×10³ cells were plated onto 8 well cell imaging coverglass chamber slides (Eppendorf). After 48 hours cells were washed 3 times with PBS and fixed with freshly prepared 4% paraformaldehyde for 20 minutes at room temperature followed by 10 minutes incubation with 20 mM Ammonium Chloride in PBS for 10 min to quench the paraformaldehyde. After 3 washes with PBS cells were stained with FILIPIN (Sigma-Aldrich) at a final concentration of 50 ug/ml for 1 h RT. After rinsing cells images were taken using confocal microscope (Zeiss LSM700) equipped with an UV laser.

Immunofluorescence and Immunohistochemistry.

Cells were plated onto 8 well cell imaging coverglass chamber slides (Eppendorf). The day after cells were treated with drugs for 48 hours cells and fixed with freshly prepared 4% paraformaldehyde. Then cells were blocked and permeabilized with 0.5% BSA and 0.1% saponin in PBS for 1 hour and incubated overnight with primary antiboby (LAMP1, Cell Signaling Technology #9091) in blocking Buffer. After incubation with Alexa-Fluor688 conjugated anti-rabbit (Thermo Fisher Scientific) for 1 hour and with DAPI (Thermo Fisher Scientific) 1 μg/ml for 5 minutes cells were washed with PBS and analyzed by confocal microscopy (Zeiss LSM700). Lysosome diameter was measured using Fiji software³⁵.

Lysosomal Assay

Lysosomal function was performed using the Lysosomal Intracellular Activity Assay Kit (Biovision) according to the manufacturer's instructions. Briefly, cells were plated onto 8 well cell imaging coverglass chamber slides, let adhere overnight and treated with drugs for 48 hours. Then medium was removed and replaced with 0.5% FBS medium with or without drugs supplemented with Self-Quenched Substrate for 2 hours in the incubator. Nuclei were stained incubating cells for 5 minutes with Hoechst 33342 dye (NucBlue® Live Ready Probes, Thermo Fisher Scientific) and washed twice with the Lysosomal Assay Buffer. Images were taken using confocal microscope with 488 nm excitation filter and fluorescence intensity was calculated using Fiji software³⁵.

Bioinformatic Analysis.

Itra activity area was calculated using GraphPad Prism v6. Cell lines with normalized activity area at least 0.8 standard deviations (SDs) above the mean were defined as sensitive to the compound, whereas those with activity area at least 0.8 SDs below the mean were defined as resistant. Cell lines with activity area within 0.8 SDs of the mean were considered to be intermediate^{36,37}. The drugZ algorithm was used to identify chemogenetic interactions from CRISPR screen as previously reported³⁴ . Venny software (//bioinfogp.cnb.csic.es/tools/venny/) was used to generate Venn diagrams. Pathway analysis on significant CRISPR screen hits (FDR and pvalue<0.05) was performed using pathDIP (//ophid.utoronto.ca/pathDIP/)³⁸. Itra inhibitory percentage activity and heat maps were generated using SynergyFinder 2.0 (//synergyfinder.fimm.fi/) and Excel. Itra/CQ synergy score was calculated based Bliss reference model. Calculation of synergy scores and generation of 2D synergy maps were obtained using SynergyFinder 2.0 as previously reported³⁹.

Phase 1 Clinical Trial

Study Design HYDRA-1 (NCT03081 702)

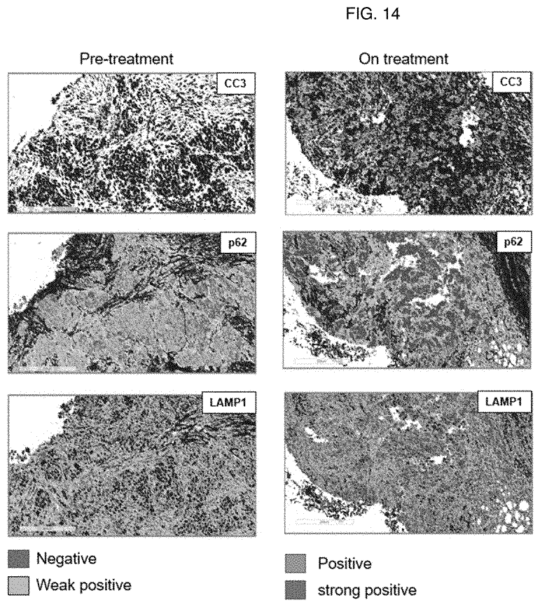

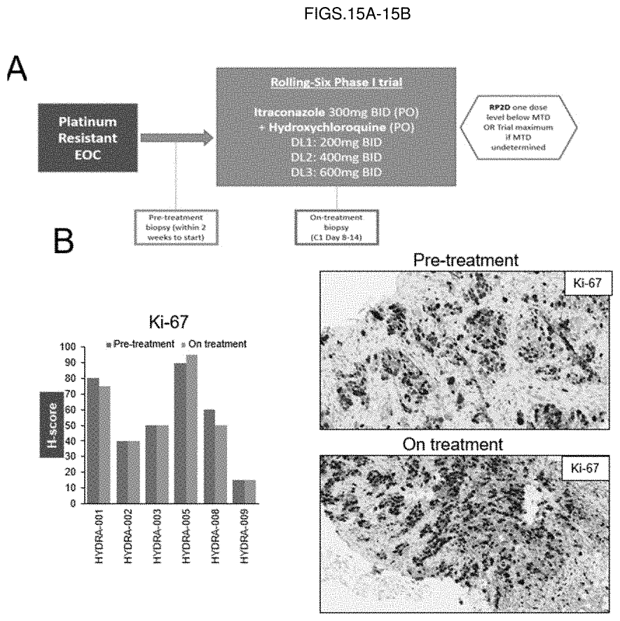

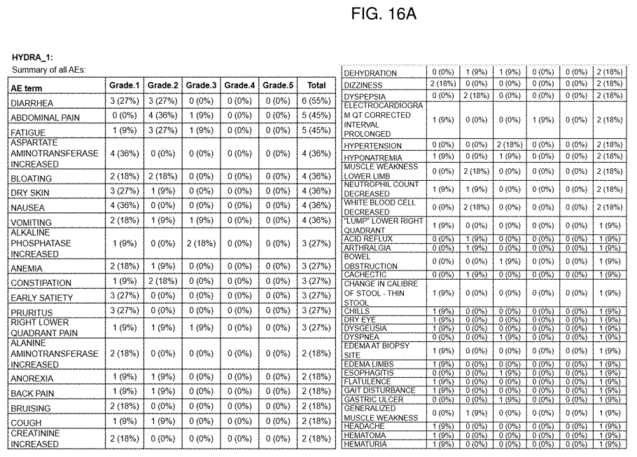

A rolling-six phase I design was used to assess the combination of Itra (PrMint-Itraconazole, itraconazole capsules, Mint Pharmaceuticals Inc., Canada) and hydroxychloroquine (HCQ), a less toxic derivative of CQ⁴⁰ (PrMint-Hydroxychloroquine, hydroxychloroquine sulfate capsules, Mint Pharmaceuticals Inc., Canada), in patients with platinum resistant or refractory EOC (FIG. 14). Women received Itra 300 mg twice daily (BID) with HCQ as per dose escalation schedule (200 mg BID in DL1; 400 mg BID in DL2; 600 mg BID in DL3), continuously in a 28-day cycle. The fixed dose of Itra was determined in previous phase II trials⁶, and since there is no known interaction based on metabolism and pharmacokinetics, a rapid dose escalation of HCQ, through a rolling-six design was incorporated⁴¹⁻⁴³. The dose-limiting toxicities (DLT) include grade≥4 anemia or thrombocytopenia, grade≥3 diarrhea or rash and grade≥2 ocular toxicity, during the initial 28 days of treatment (Protocol in Supplementary Material). The calculated sample size for the phase I trial was between 6 and 18.

Tumour assessment occurred every 8 weeks (+/−1 week) by CT scan. Toxicity was assessed by Common Terminology Criteria (CTCAE) version 4.0. Pre- and on treatment biopsies (on cycle 1, day 8 to 14) were mandatory.

The primary objective was establishment of maximum tolerated dose; secondary objective was objective response rate (ORR) by RECIST v1.1 criteria, and progression free survival (PFS). Exploratory objectives were assessment of apoptosis, proliferation and angiogenesis pathways.

Patients

Enrolled patients were ≥18 years old with an ECOG performance status of 0-1, and platinum resistant or refractory EOC. Eligible patients had adequate blood and marrow function (hemoglobin ≥90 g/L, absolute neutrophils ≥1.5×109/L, platelets ≥100×10⁹/L, bilirubin within normal limits, AST ≤2.5×institutional upper limit of normal, ALT, serum creatinine within normal limits or creatinine clearance≥60 ml/min). Women on CYP3A4 inhibitors/inducers or on statins were ineligible. Patients with a known G6PD deficiency or a known retinopathy were ineligible. Patients with a clinical indication for treatment with Itra or HCQ, and those with intestinal malabsorption or active bowel obstruction were excluded. There was no limitation regarding prior number of lines of therapy.

Tissue Pharmacokinetic Analyses

Tumour Itra and HCQ concentrations were determined by a HPLC-MS/MS method with itraconazole-D9 and hydroxychloroquine-D4 as the internal standard. The HPLC system was interfaced to a SCIEX TRIPLE QUAD 6500+ mass spectrometer operating in the negative electrospray ionization mode. Data collection, peak integration, and calculation were performed using Analyst® 1.7 software.

Exploratory Objective Methods

RNA was isolated from FFPE tissue using the QIAGEN All-Prep DNA/RNA/miRNA universal Co-Isolation kit and sequenced using the using the TruSeq Stranded Total RNA kit with Ribo-Zero Gold from Illumina followed by sequencing using an Illumina Nextseq500 sequencer. RNA-Seq reads were aligned using STAR aligner v2.6.0c (https://doi.org/10.1093/bioinformatics/bts635). Gene expression was quantified using RSEM v1.3.0. Gene fusions were detected using StarFusion v1.5.0 (https://doi.org/10.1101/120295).

Immunohistochemistry (IHC) was performed using the BenchMark XT automated stainer (Ventana Medical System) with antigen retrieval (CC1, Tris/Borate/EDTA pH8.0, #950-124) for 64 minutes. The Ki-67 (clone: MIB1, M7240, Dako) dilution was 1:100 with 60-minute incubation. The anti-CC3 (D175, 9661, Cell Signaling Technology) dilution was 1:500 with 32-minute incubation. The anti-LAMP1 (9091, Cell Signaling Technology) dilution was 1:1000 with 60-minute incubation. The anti-P62 (2C11, WH000887M1, Sigma) dilution was 1:4000 with 32-minute incubation. The Ventana's ultraView Detection Kit (#760-500) was utilized and the slides were counterstained with Gill modified hematoxylin.

The H-score method was used to assess immunoreactivity for CC3, p62 and LAMP1. In brief, the H-score is obtained by the formula: 3×percentage of strong staining+2×percentage of moderate staining+percentage of weak staining, resulting in a range from 0 to 300. The Ki-67 proliferation index was visually estimated to the nearest 5% increment.

IHC slides were scanned at a 20× magnification using the Aperio Scanscope AT2 whole Slide Scanner (Leica) and IHC images and heat map analysis were obtained using Aperio ImageScope software (Leica).

Statistical Analysis

All dose response graphs were generated using GraphPad Prism v6. Histograms and Swimmer plots were generated using R software. Patient clinical features and response details were described using summary statistics, such as medians, ranges, frequencies and proportions. PFS analysis was conducted using the Kaplan-Meier method for all patients. Median and confidence interval were reported to assess PFS. Treatment related toxicity was evaluated using frequencies and proportions of adverse events based on severities and attributions. Statistical significance of differences among groups in lysosomal pattern, lysosomal function and TFEB nuclear translocation were assessed using the one-way analysis of variance (ANOVA) in which a p value≤0.05 is considered to be statistically significant. Pathway analysis on CRISPR screen results using PathDIP software uses Fisher's exact test and corrects raw P-values for multiple hypothesis testing based on Bonferroni and false discovery rate (BH method) methods³⁸.

Results

Evaluation of Therapeutic Potential of Itraconazole in Ovarian Cancer

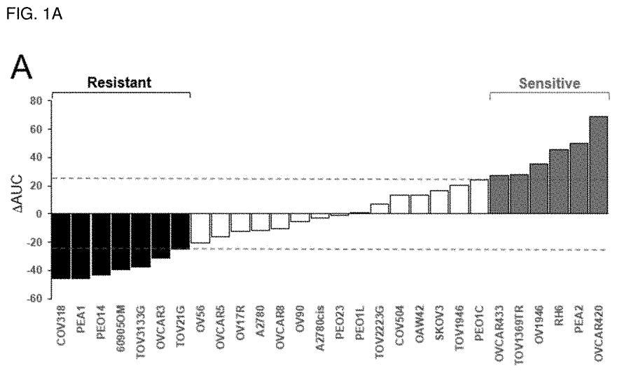

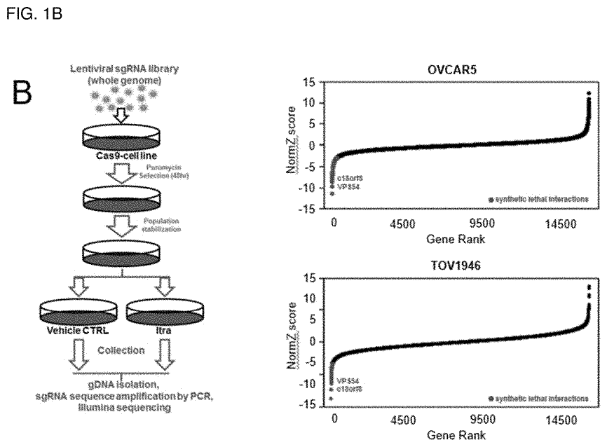

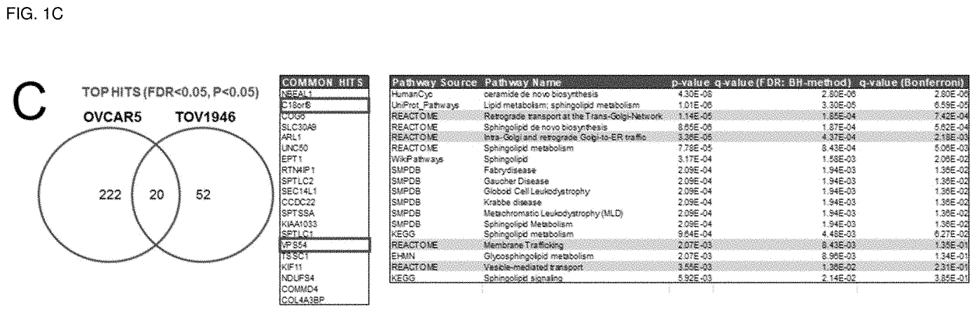

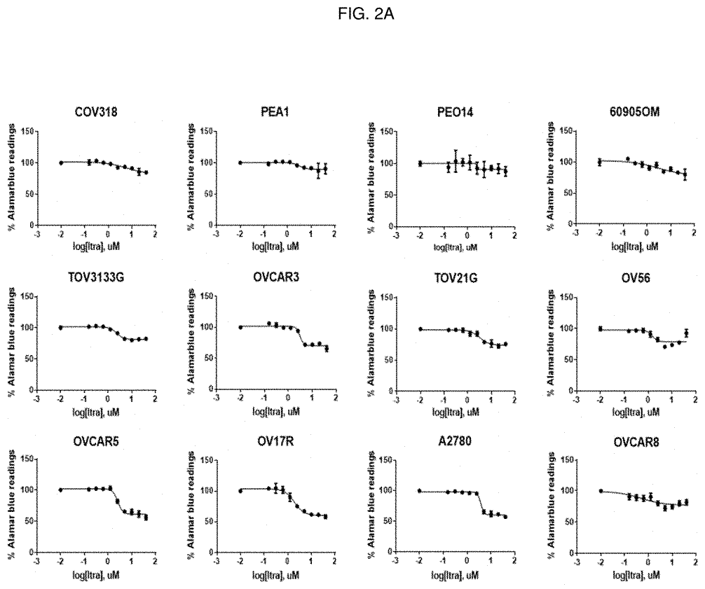

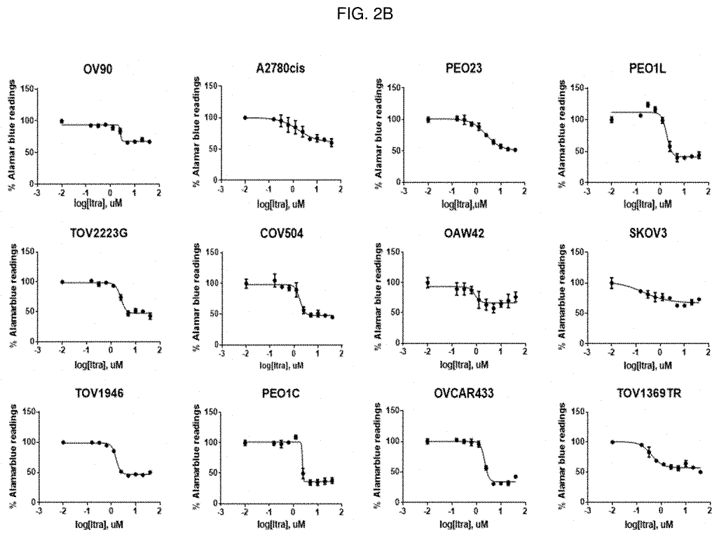

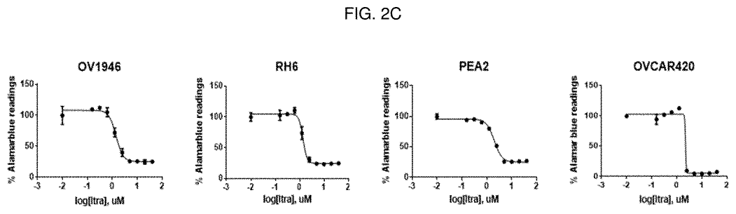

To explore the therapeutic potential of Itra in the treatment of EOC, we screened a panel of 28 cells lines over 5 days of exposure. Quantitative scoring of differential Itra sensitivity was calculated using the activity area method (corresponding to the area over the drug response curve) as previously reported^{36,37,44} (FIG. 1A, FIGS. 2A-2C), and a threshold of 0.8 SD on the mean value was employed to identify sensitive and resistant cell lines, resulting in 7 resistant, 15 intermediate and 6 sensitive cell lines³⁷. These data suggest that Itra has an effect in a subgroup of ovarian cancer cells.

Lysosomal Compartments as Important Regulators of Itraconazole Resistance Identified Through a CRISPR Screen

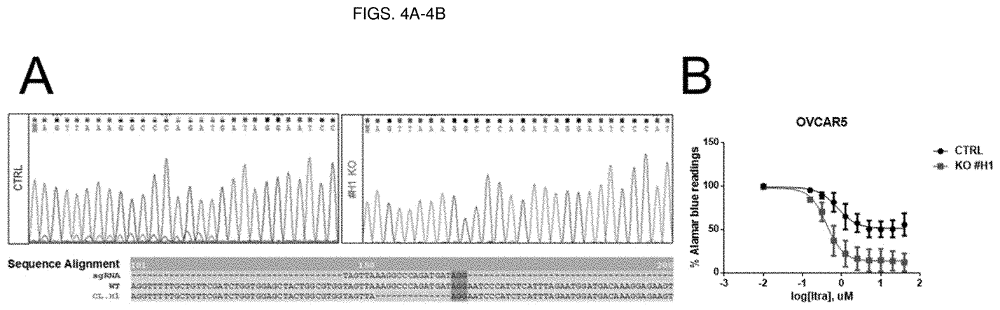

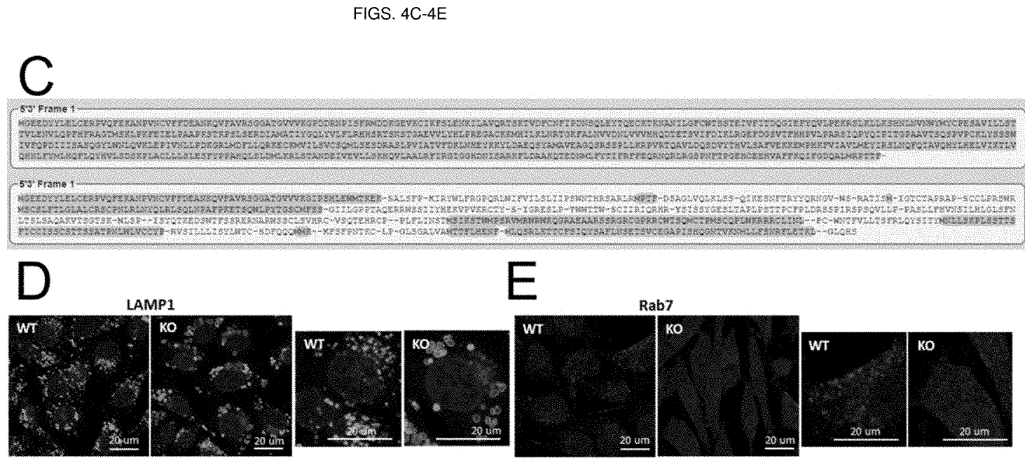

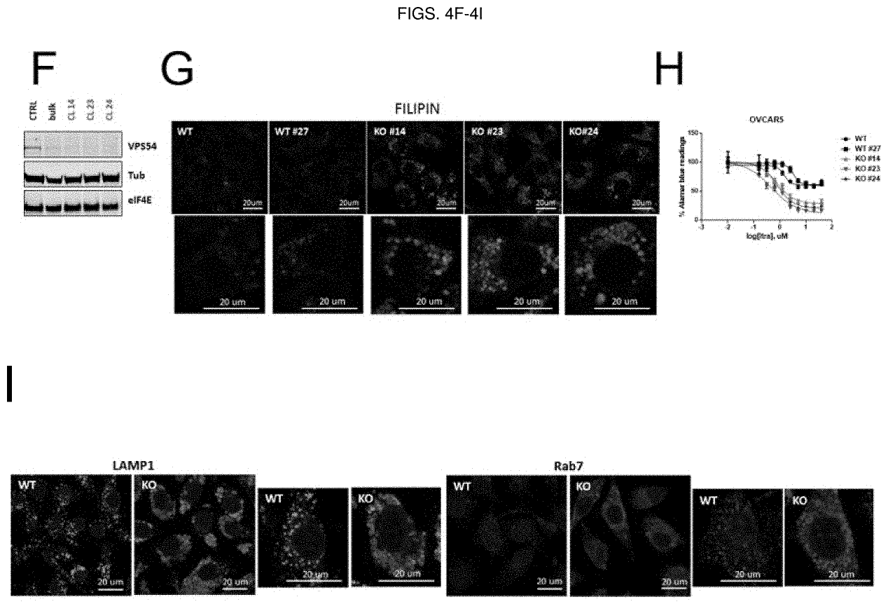

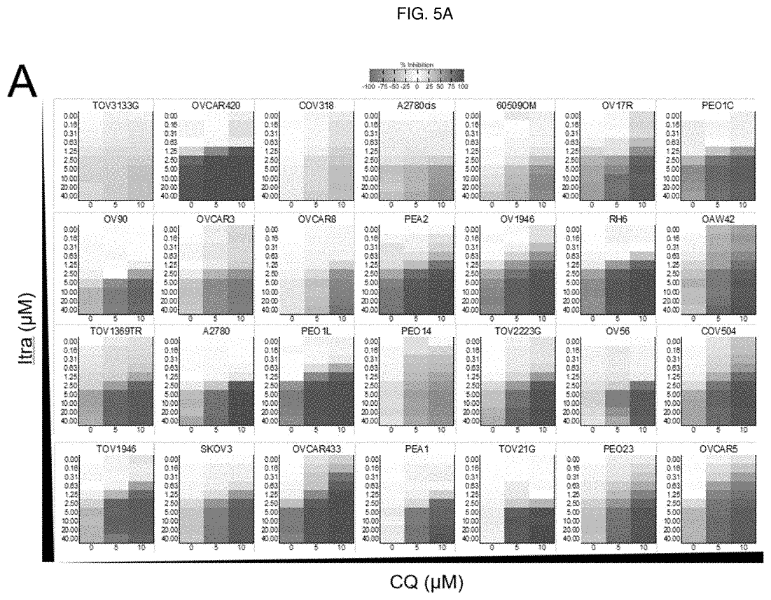

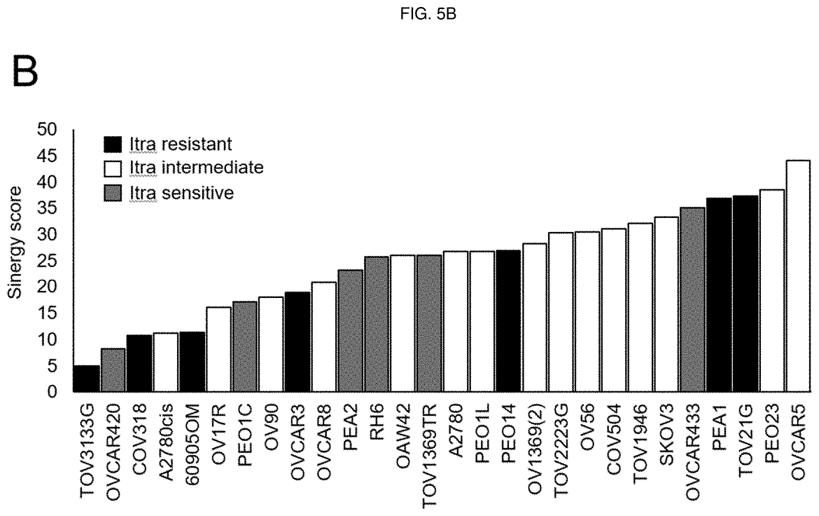

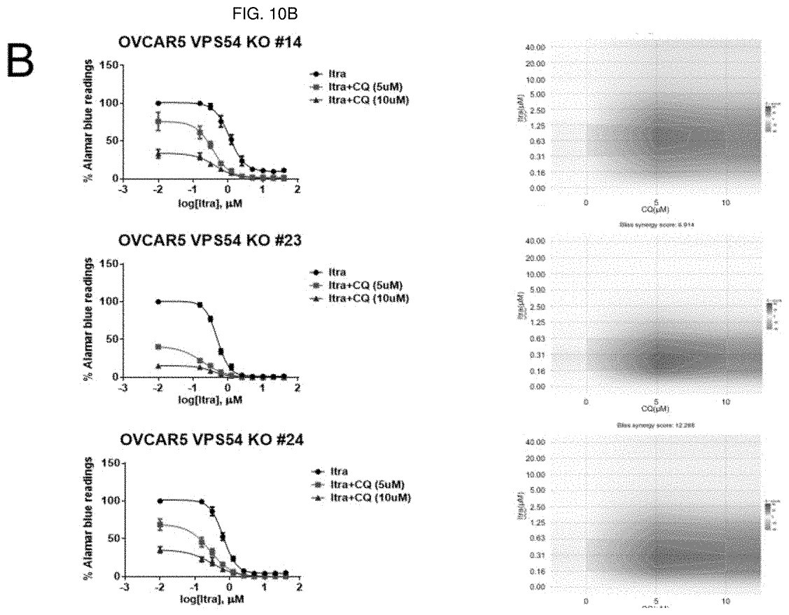

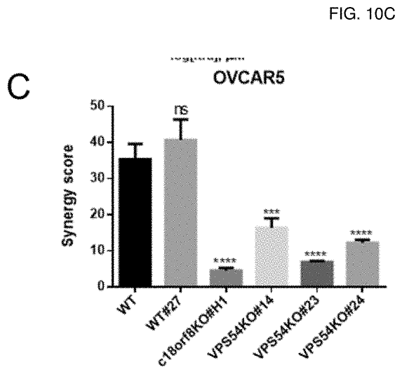

To identify genes and pathways involved in resistance to Itra, we performed a whole genome sensitizing CRISPR screen in two cell lines (TOV1946 and OVCAR5). We stably expressed Cas9 (FIG. 1B, FIGS. 5A-C) and verified its activity with appropriate controls (FIGS. 4A-C). Infected cells were treated with non-toxic concentrations of Itra (1 μM) (FIG. 1B, FIGS. 4A-C). Analysis of the synthetically lethal hits using DrugZ algorithm³⁴ with a false discovery rate (FDR) and p value<0.05 reported 76 genes for TOV1946 and 242 genes for OVCAR5. Twenty genes were common in the two screens. To identify common pathways in the two screens, we carried out a pathway analysis³⁸ and found that many genes were related to vesicular trafficking and the dynamics between the transgolgi network (TGN) and late endosomal/lysosomal compartments (LE/L) (FIG. 10). To validate our CRISPR screen results we selected two genes involved in these pathways for further analysis, VSP54 and c18orf8, previously reported to have a role in lysosomal biology and dynamics.

C18orf8, also known as MIC1 or RMC1, is a component of CCZ1-MON1 complex that is required for recruitment of Rab7 at LE/L and lysosomal maturation. Knockdown of c18orf8 has been shown to impair lysosomal maturation, induce lysosomal enlargement and dysfunction and inhibit autophagy⁴⁵. Consistent with the CRISPR screen, stable knockout of c18orf8 (FIG. 4A) strongly sensitized OVCAR5 to Itra (FIG. 4B) and dramatically increased lysosomal size and decreased maturation as shown by a diffuse cytoplasm staining in knockout cells compared to a dotted like in control conditions (FIGS. 4D, 4E).

Vacuolar protein sorting-associated protein 54 (VPS54) is a component of the Golgi-associated retrograde protein (GARP) complex, which regulates retrograde transport from late endosomes to TGN and is particularly important for recycling of mannose 6 phosphate receptor (M6PR) required for proper delivery of lysosomal proteins⁴⁶. Knockdown of GARP VPS subunits leads to cholesterol accumulation in the lysosomes as a reflection of impaired delivery to the LE/L of cholesterol transporters and lysosomal enlargement and dysfunction. Similar to knockout of c18orf8, knockout of VPS54 lead to increased sensitivity to Itra, cholesterol accumulation (as measured by filipin staining) and lysosomal enlargement (FIGS. 4F, 4G, 4H). Absence of VPS54 did not influence lysosomal maturation as knockout cells showed a Rab7 dotted pattern in enlarged vesicular structures (FIG. 4I). Taken together, these results indicate that efficient lysosomal flux and function are important regulators of Itra resistance and impairment of these pathways strongly sensitizes ovarian cancer cells to this drug.

Synergistic Effects of Itraconazole and the Lysosomotropic Drug Chloroquine

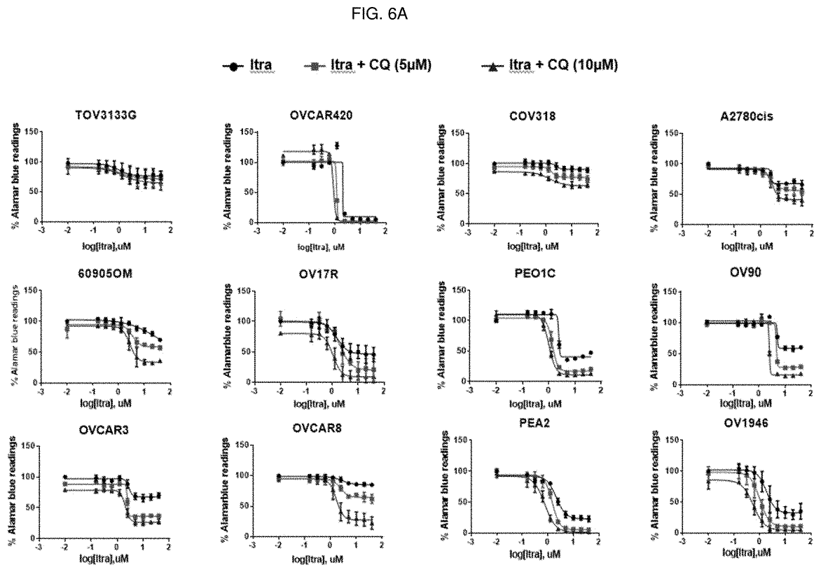

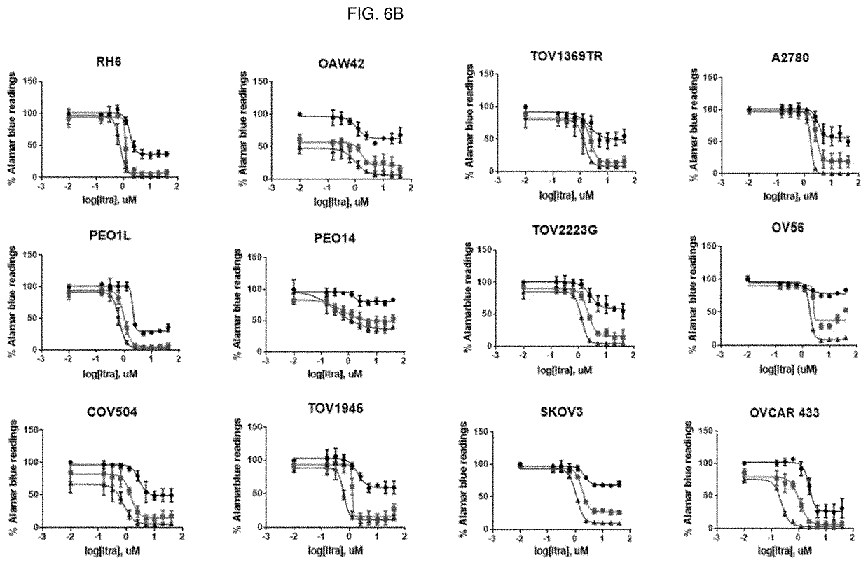

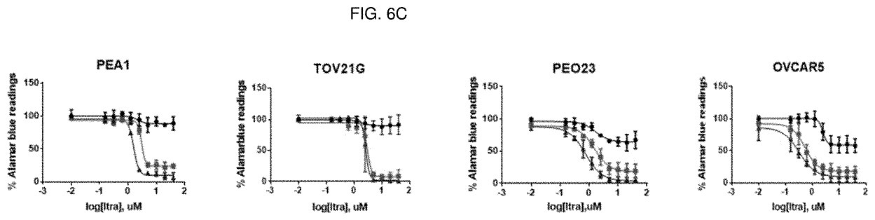

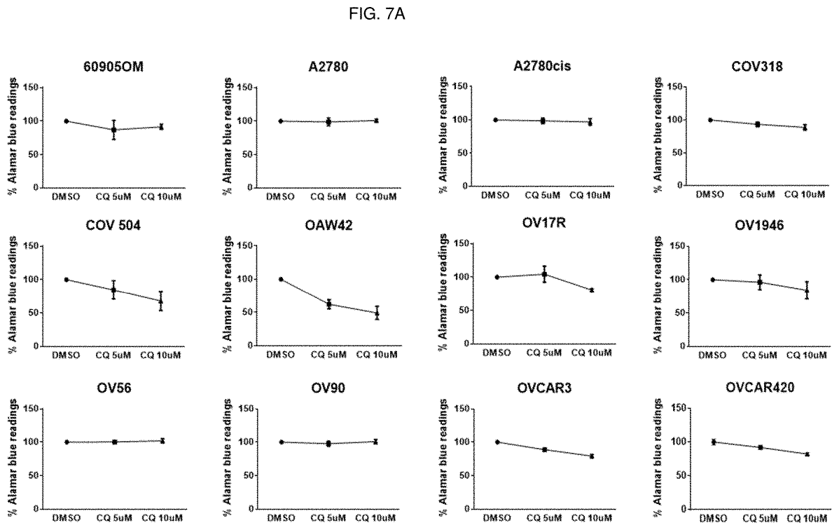

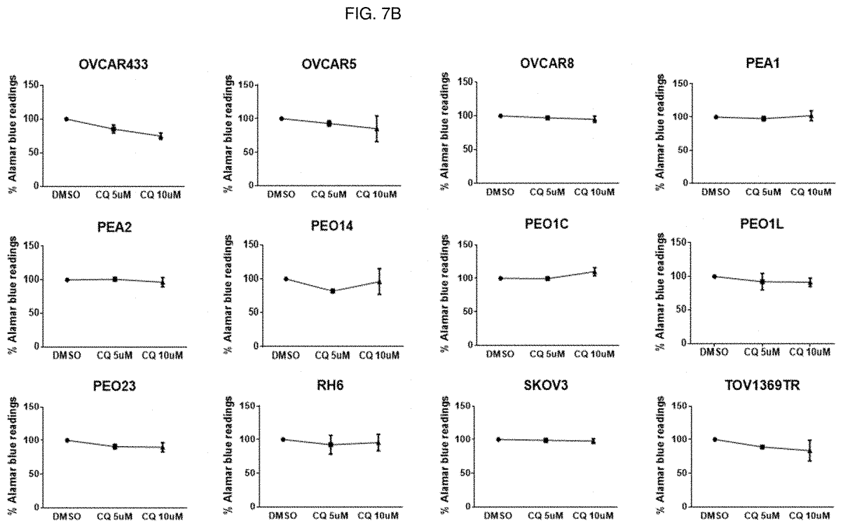

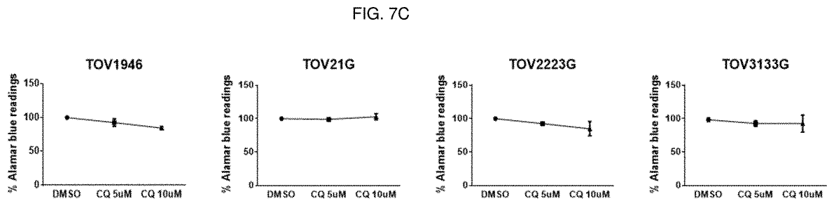

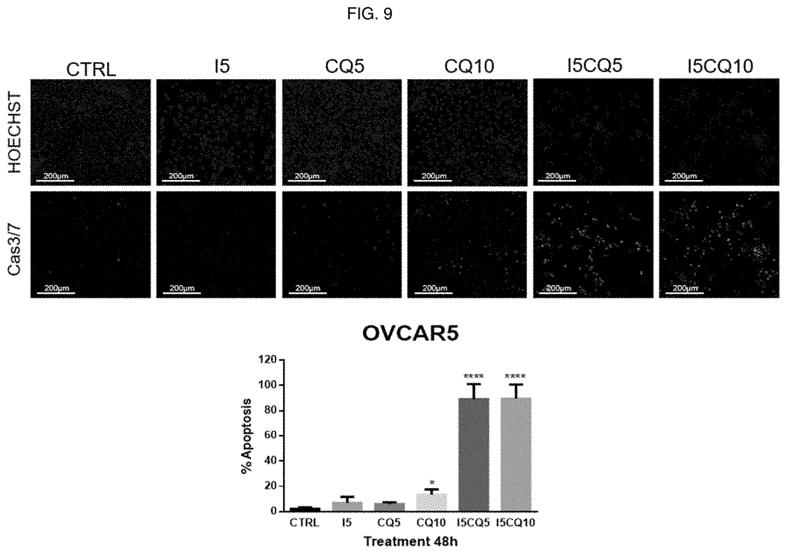

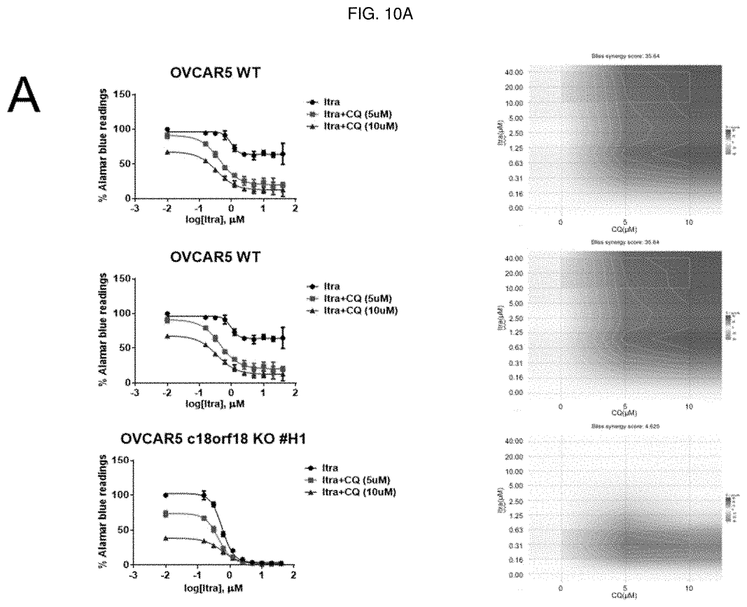

We next postulated that drugs capable of phenocopying the effects of knocking out these two genes would result in an increased sensitivity to Itra. The antimalarial drug chloroquine (CQ) and its derivative, hydroxychloroquine (HCQ) are drugs that have been repurposed in several cancer related clinical trials for their anti-inflammatory properties and for targeting autophagy at lysosomal level³¹. Similar to VPS54 knockdown, CQ was shown to impair recycling of M6PR from late endosomes to TGN and to induce lysosomal enlargement and dysfunction⁴⁷. We thus tested and quantified the effects of combining Itra with CQ in the above used cell lines via the Bliss model measuring synergy⁴⁸. Similar, to what we observed for Itra alone, we found a different spectrum of activity to Itra/CQ combinations (FIGS. 5A-B, FIGS. 6A-8C). Interestingly, TOV1 946 and OVCAR5 were among the cells that showed the highest levels of synergy, showing an increase in sensitivity similar to previously reported for c18orf8/VPS54 knockout^{45,46} (FIGS. 5A-B, FIG. 6). Moreover, cell lines such as TOV21g exquisitely resistant to Itra alone as reported by low Itra activity area (FIGS. 1A-C), become very sensitive to the combinations with Itra and CQ being the second cell line ranked for synergy score (FIGS. 5A-B, FIG. 6). As expected, the Itra/CQ combined effect was observed but significantly reduced in c18orf8 and VPS54 knockout cell lines compared to controls (FIG. 9).

To better understand the biological effects of co-treatment with Itra and CQ, we examined lysosomal pattern, function and stress response in cells that displayed high levels of Bliss defined synergy (FIGS. 5A-B).

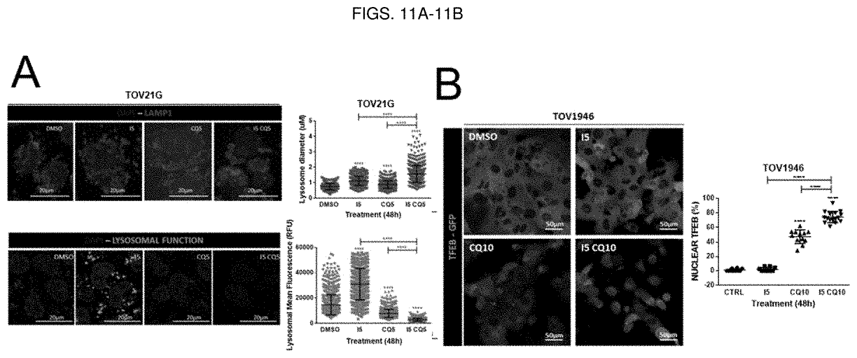

Lysosomal pattern and morphology was assessed by examining the Lysosomal-associated membrane protein 1 (LAMP1), one of the most abundant lysosomal membrane proteins that generally localizes to lysosomes and late endosomes^{24,27}. In TOV21g cells, we observed a combined effect in lysosomal enlargement (FIG. 11A) induction in parallel with a reduced lysosomal function (FIG. 11B). In particular, when evaluating lysosomal function using a the lysosomal Intracellular activity assay based on the employment of selfquenched substrate that become fluorescent once internalized and exposed to the lysosomal enzymatic activity⁴⁹, we observed an increased fluorescence (reflective of lysosomal function) in cells treated with Itra alone and a slight but significant reduction in cells treated with CQ. However, in cells co-treated with Itra and CQ fluorescence was barely detectable. Taken together these results indicate that the two drugs have a combined effect at lysosomal level, by reducing its degradative potential and affecting the ability to breakdown molecules, resulting in an aberrant accumulation of endocytic cargo that leads to lysosomal enlargement.

These results were corroborated by the effects obtained analyzing lysosomal stress response in cells co-treated with Itra and CQ (FIG. 11C). Member of the microphtalmia (MiT) family (TFEB, TFEB, TFE3) are transcription factors activated in starvation or lysosomal dysfunction conditions. Once activated, they translocate to the nucleus where they promote the expression of a network of genes related to lysosomal function and biogenesis⁵⁰. At a concentration of 10 μM CQ, we observed TFEB nuclear translocation in about 50% of cells; however, when we combined the two drugs, nuclear translocation increased significantly to 90%, indicating a substantial augmentation of lysosomal stress and biogenesis by the affected cells.

Validation cohort—Phase 1 clinical trial (HYDRA-1, NCT03081702)

Baseline Demographics

Between 2017 and 2019, 13 patients were enrolled and two withdrew consent to participate prior to the initiation of the therapy. Median age was 54 (44, 77). At least one cycle of treatment was administered to 11 women; five in dose-level one (DL1), three in dose-level two (DL2) and three in dose-level three (DL3). Out of the 11 patients, ten were evaluable for efficacy. Histology was high grade (91%, N=10) and low grade (9%, N=1) serous ovarian cancer, and median prior lines of systemic therapy was seven. Median number of prior lines of treatment was seven (range 3-9).

Safety and Clinical Activity

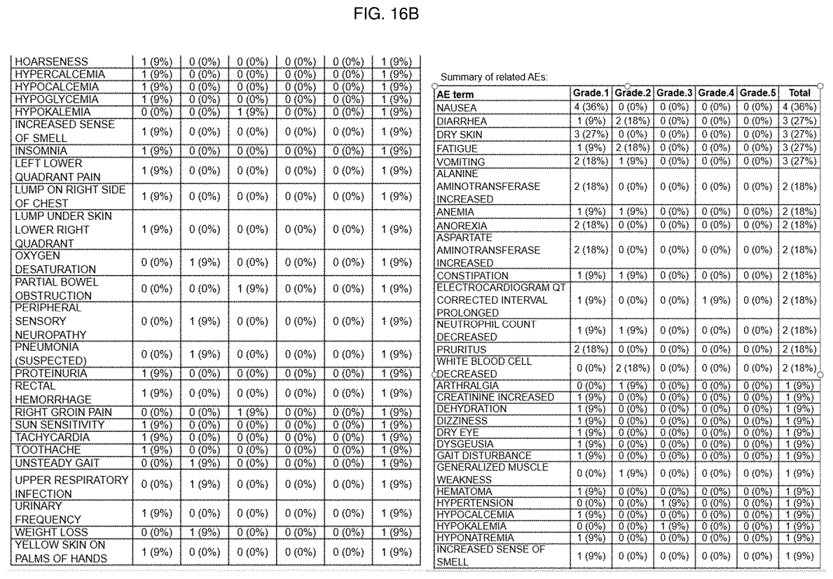

The most frequent treatment related toxicity was nausea in 36% of the patients (grade 1), followed by diarrhea, vomiting, fatigue and dry skin in 27% of patients (grade 1-2) (FIG. 16). Other grade≤3 adverse events were grade 3 hypokalemia and grade 4 QTc prolongation (in one patient, DL3). A DLT was seen in DL2, grade 3 hypertension, which was manageable with medication. There were no treatment discontinuations due to toxicity. Treatment was held in one patient (DL1) due to intolerable fatigue and muscle weakness. The recommended phase 2 dose (RP2D) was ltr 300 mg BID and HCQ 600 mg BID (DL3).

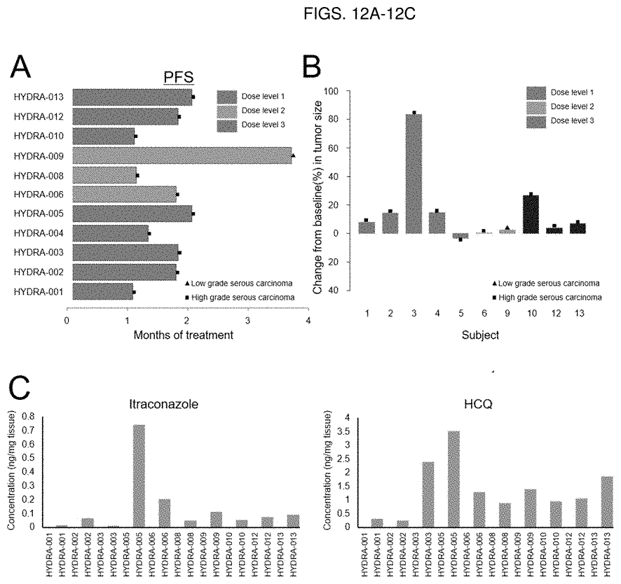

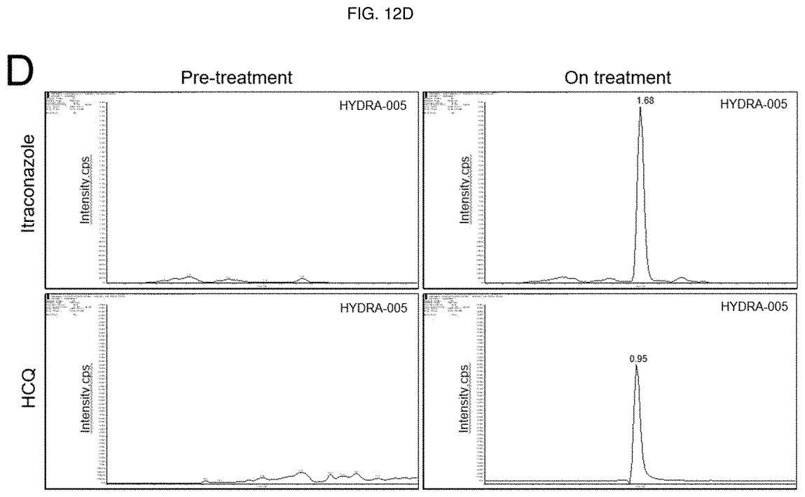

No objective responses were seen and one patient with low grade serous histology had stable disease for 3.7 months. Median PFS was 1.6 months (95% Confidence Interval 1-1.7 months) (FIGS. 12A,B). Median cycles of treatment per patient was two (range 2-4).

The treatment combination was feasible.

Pharmacokinetics

Pre and on-treatment biopsies were available for ten patients. The concentrations of

Itra and HCQ were detectable in all patients in the post-treatment samples and none of the pre-treatment samples. The highest concentrations of both drugs were detected in one patient, HYDRA-005 that received treatment at DL1, with an intratumor concentration of 0.745 ng/mg of tissue for Itra and 3.5 ng/mg tissue for HCQ (FIGS. 12C,D).

IHC and RNA Sequencing

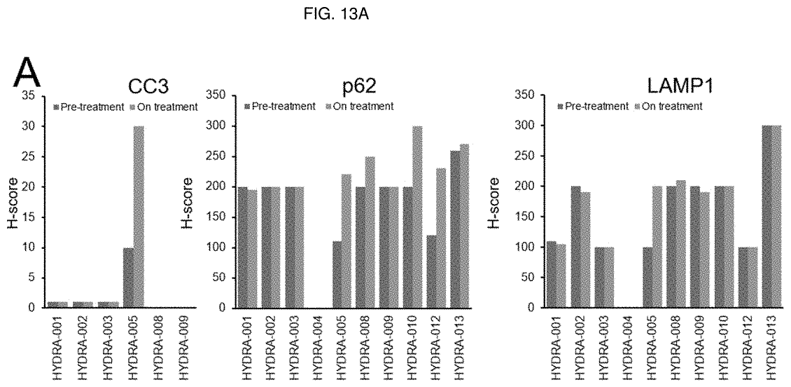

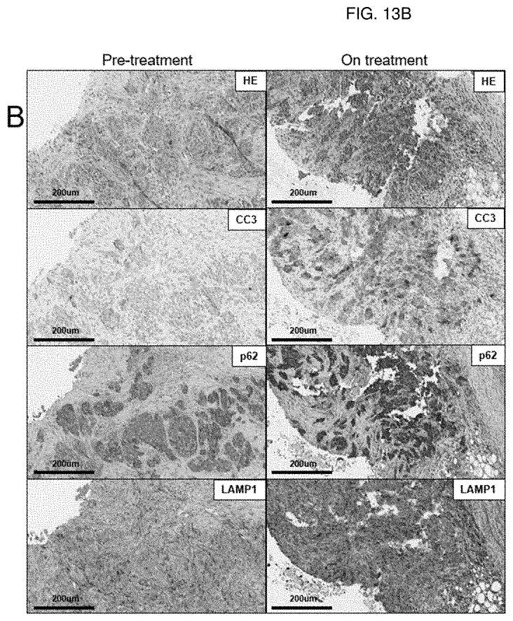

Pre- and on-treatment biopsies were available for IHC analysis in ten patients. No significant changes were detected in the overall population in terms of IHC markers (autophagy, apoptosis, DNA damage and lysosomal markers), morphology, mitosis and proliferation index (Ki-67). To explore the pharmacodynamic effect of the drugs we stained the tumour sections for LAMP1 (a marker of late endosomes/lysosomes), as well as p62 (a marker that correlates with impaired autophagy). The expression of lysosomal marker, LAMP1, increased in one patient (HYDRA-005) in the on-treatment biopsy compared to the baseline biopsy. This patient also had an increase in the autophagy related protein (P62), previously shown to accumulate in tumours treated with CQ^{51,52}, and apoptosis related protein (cleaved caspase 3, CC3) (FIGS. 13A-B, FIGS. 10A-C). Non-significant differences were observed in Ki-67 staining in tumour tissues (FIG. 14B). This patient had higher intra-tumour drug concentrations, and a decrease in size in the target lesions (FIGS. 12C-D); however, there was no correlation with clinical benefit.

Single-sample gene-set enrichment analysis using RNA sequencing data did reveal any change from pre- to post-treatment samples in relevant pathways including apoptosis, angiogenesis, cholesterol metabolism, lysosomal-autophagy and Pl3K-AKT-mTOR pathways pre-treatment and on-treatment samples.

Discussion:

Despite optimal front-line treatment, recurrence rates in stage III/IV disease are high, with median overall survival in platinum resistant setting estimated at only 12 months⁴. Platinum resistance is multifactorial and evolves over time in response to selective pressure of treatment⁴. By repurposing two U.S. Food and Drug Administration (FDA) approved drugs, the antifungal drug Itra and the antimalarial drug HCQ, we explored lysosomal homeostasis as a potential therapeutic target in the treatment of EOC.

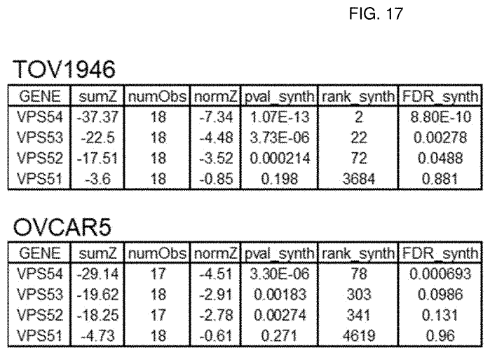

Based on previous studies showing the beneficial effects of Itra in patients with EOC^{5,22,23,53}, we investigated the therapeutic potential of Itra across a wide panel of EOC cell lines and identified synthetically lethal gene deletions of genes involved in vesicular trafficking and dynamic exchanges between TGN and lysosomes. We validated hits from our CRISPR screen known to affect lysosomal maturation and function, VPS54 and c18orf8 to show in both cases, knock-out cell lines resulted in an abnormal lysosomal morphology as characterized by lysosomal enlargement. This change in lysosomal pattern is frequently associated with acidification defects, aberrant accumulation of endocytic cargo, lysosomal dysfunction and observed in many lysosomal storage disease⁵⁴. Most importantly, knockout of VPS54 and c18orf8 dramatically increased sensitivity to Itra, confirming results obtained from the CRISPR screen. We also saw evidence of synthetic lethal interactions in other members of the GARP complex, a heterotetrameric tethering factor consisting of four subunits, VPS51, VPS52, VPS53 and VPS54. Interestingly, other GARP subunits resulted among the top hits in the CRISPR screen as significantly associated with Itra sensitization, with VPS53 and VPS52 subunits having an FDR and p value<0.05 in TOV1946 and a FDR close to 0.1 and p value<0.05 in OVCAR5 (FIG. 17).

The GARP complex has been reported to be recruited to the trans Golgi network by Rab6 in mammalian cells. Depletion of VPS subunits results in a block in transport of a several cargos, including the cation-independent mannose-6-phosphate receptor (CI-MPR), indicating that GARP has a general role in endosome-to-TGN transport pathways^{46,55}.

The antimalarial drug CQ, and its derivate used in therapy HCQ, are lysosomotropic compounds sharing similar chemical structures and mechanisms of acting as a weak base³¹. As liphophilic molecules with weak base properties they can readily diffuse across cell membranes via passive diffusion and upon entry into acidic lysosomes and late endosomes, undergo protonation and are hence entrapped in lysosomes in their cationic state^{56,57}. The pH gradient between the lysosomal lumen and the cytosol drives hyper-accumulation of these drugs leading to high intra lysosomal concentration²⁹. Prolonged treatments with CQ/HCQ and other lysosomotropic cationic drugs induce the formation of large vacuole-like structures that are formed following osmotic imbalance possibly causing water influx and lysosomal swelling⁵⁸. Moreover, the increase in lysosomal pH impairs the release of lysosomal proteins from Mannose 6 Phosphate receptors that normally are released at low pH and thus a reduced vesicular recycling to TGN⁴⁷. Thus, these drugs have been shown to phenocopy the effects observed with knockout of VPS54 and c18orf8⁴⁷.

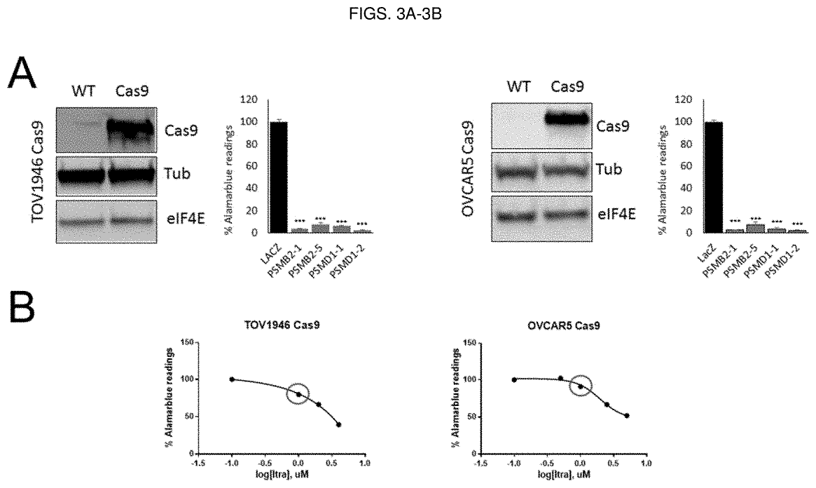

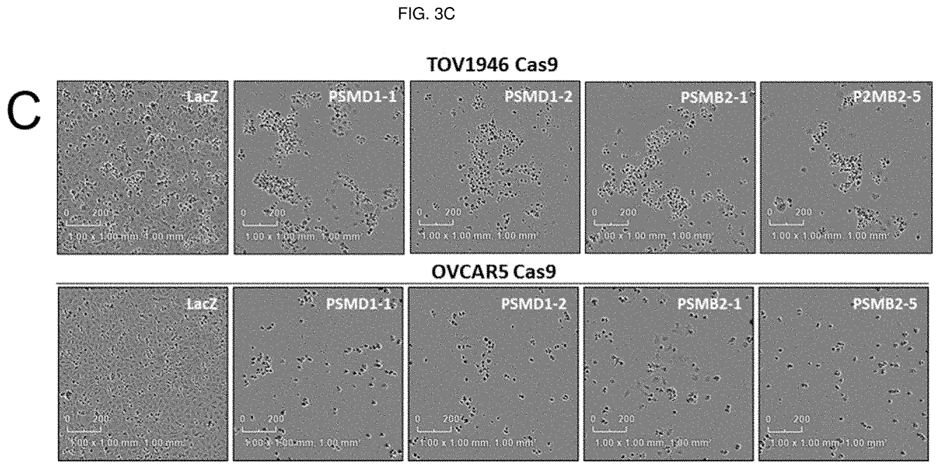

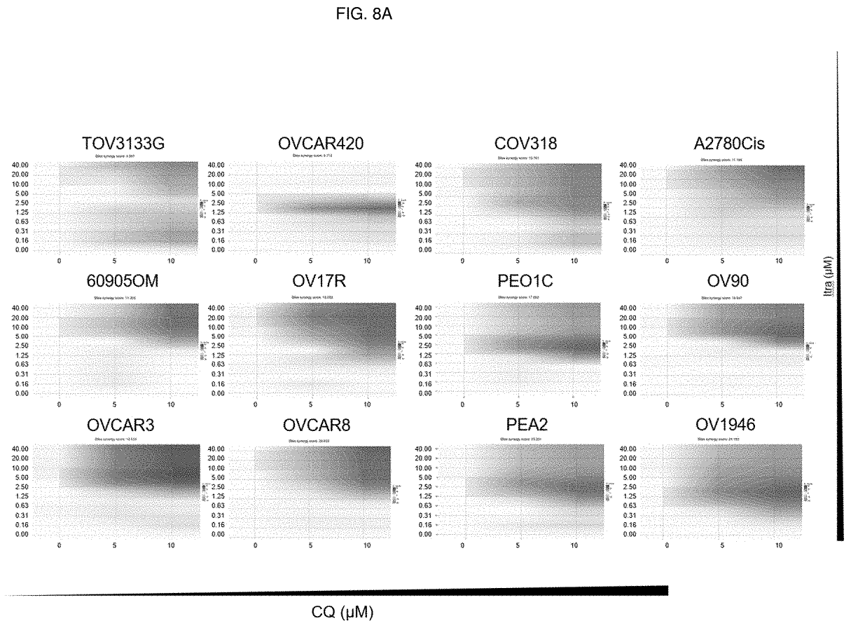

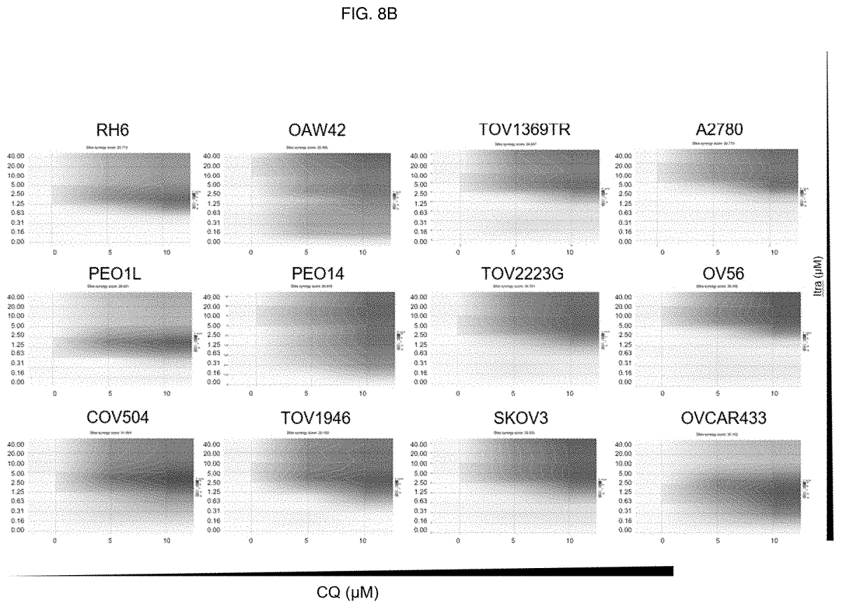

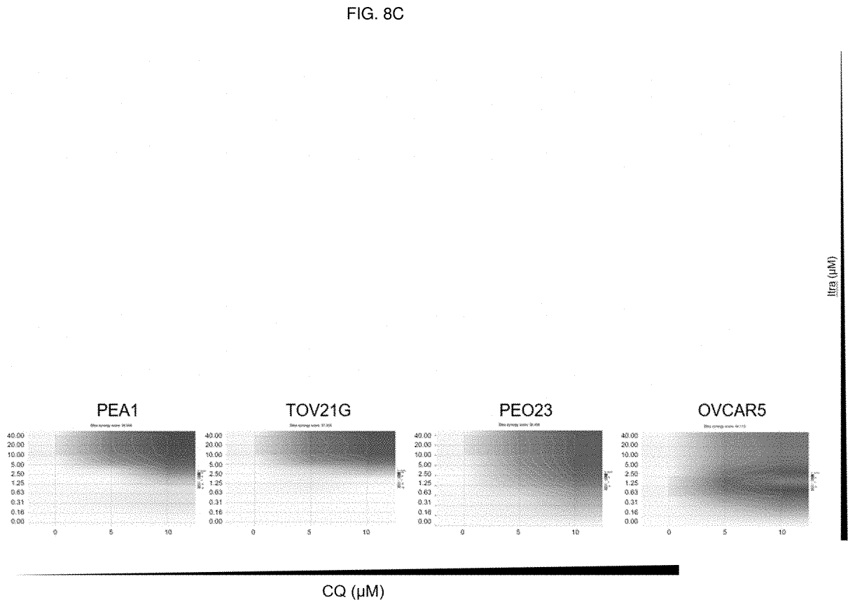

This combination treatment was explored to all the cell lines previously tested with Itra and interestingly we observed a synergistic effect in several cell lines, including some that previously showed high resistance to Itra (FIGS. 5A-B, FIG. 3, FIG. 8). These data were corroborated by a (I) combined effect of Itra with other potent lysosome targeting agents like Bafilomycin A and Concanamycin A)⁵⁹ (data not shown) and (ii) in cells stably expressing fluorescent TFEB, we observed a significant increase in nuclear localization, and thus an increase of a lysosomal stress condition, in presence of both drugs compared to single treatments. A recent paper by Weber et al., described the importance of iron and cholesterol metabolism in combination with lysosomal de-acidification, yet we are unaware of any literature linking itraconazole and iron metabolism. It is however possible that sensitive cell lines have a greater degree of cholesterol dependency in the presence of both lysosomal disruption and cholesterol antagonism.

To gather more knowledge about the therapeutic potential of this drug combination translated to a clinical scenario, we then conducted a phase I dose-escalation study assessing the combination of Itra and HCQ in platinum resistant or refractory EOC. This combination was safe at the determined dose but did not lead to anti-tumour activity with the combination. Interestingly, one of the patients in DL1 (HYDRA-005) had evidence of higher drug concentrations in tumour and increase in the relevant pathways including apoptosis, autophagy and lysosomal markers with evident signs of lysosomal enlargement, per IHC. These data suggest that Itra/CQ combination can be effective in conditions where both drugs accumulate at a minimum effective concentration in tumour tissue; however, although target lesions decreased in size, this did not correlate with clinical benefit as the patient had progressive disease with formation of new lesions. The limitations of our study include that given that it was a pilot study only a small number of patients were enrolled. Women had predominantly high grade serous ovarian carcinoma and were heavily pre-treated with a median of seven prior lines. In addition, (excluding patient HYDRA-005), generally the tissue concentration of Itra was insufficient to achieve an anti-tumour effect. Making the assumption that 1 μg of tissue correspond to a volume of 1 μl, we can estimate a concentration of maximum of 1 μM for Itra in patient HYDRA-005, and a range of HCQ of 5-10 μM. By comparing these concentrations to the in vitro experiments, it becomes clear that Itra concentration in tissues may be the limiting factor for a proper efficacy of the two drugs and that a strategy aimed at augmenting Itra concentration in the tissues could provide better result. Previous retrospective studies in EOC suggested a signal of activity of Itra in combination with chemotherapy in clear cell histology²³. Advanced clear cell tumours are an aggressive chemo-resistant subtype of EOC,³ whose biology often involves canonical signaling pathways such as e PI3K/Akt signaling pathway are is the major molecular aberrations observed in 40% of patients³. Itra can exert anti-tumour effect by both suppressing the PI3K signaling pathway, being a potential therapeutic alternative to explore in this aggressive subtype of EOC as well as potentially releasing cytotixic free cholesterol from lysosome disruption. Remarkably, one of the cell lines that showed the highest synergy of Itra in combination with CQ was a clear cell carcinoma cell line indicating that this clinical subgroup could be particularly sensitive to lysosomal targeting. This hypothesis is supported by parallel in vitro studies in renal clear cell carcinoma cell lines where Itra and CQ cytotoxicity synergy was observed (data not shown). Unfortunately, one of the limitations of our study is that patients with clear cell histology were not enrolled in the clinical trial

### REFERENCES

1. Noone A M Krapcho M, Miller D, Brest A, Yu M, Ruhl J, Tatalovich Z, Mariotto A, Lewis D R, Chen H S, Feuer E J, Cronin K A, H. N. SEER Cancer Statistics Review, 1975-2015, National Cancer Institute. Bethesda, Md., https//seer.cancer.gov/csr/1975_2015/, based November 2017 SEER data submission, posted to SEER web site, April 2018.

2. Ramalingam, P. Morphologic, Immunophenotypic, and Molecular Features of Epithelial Ovarian Cancer. Oncology (Williston Park). 30, 166-176 (2016).

3. Lheureux, S., Braunstein, M. & Oza, A. M. Epithelial ovarian cancer: Evolution of management in the era of precision medicine. CA. Cancer J. Clin. 69, 280-304 (2019).

4. McMullen, M., Madariaga, A. & Lheureux, S. New approaches for targeting platinum-resistant ovarian cancer. Semin. Cancer Biol. (2020). doi:10.1016/j.semcancer.2020.08.013

5. Pantziarka, P., Sukhatme, V., Bouche, G., Meheus, L. & Sukhatme, V. P. Repurposing Drugs in Oncology (ReDO)-itraconazole as an anti-cancer agent. Ecancermedicalscience 9, 521 (2015).

6. Antonarakis, E. S. et aL Repurposing itraconazole as a treatment for advanced prostate cancer: a noncomparative randomized phase II trial in men with metastatic castration-resistant prostate cancer. Oncologist 18, 163-173 (2013).

7. Tsubamoto, H. et al. Repurposing itraconazole as an anticancer agent. Oncol. Lett. 14, 1240-1246 (2017).

8. Lam, A., Hoang, J. D., Singleton, A., Han, X. & Bleier, B. S. Itraconazole and clarithromycin inhibit P-glycoprotein activity in primary human sinonasal epithelial cells. Int. Forum Allergy Rhinol. 5, 477-480 (2015).

9. Aftab, B. T., Dobromilskaya, I., Liu, J. O. & Rudin, C. M. Itraconazole inhibits angiogenesis and tumor growth in non-small cell lung cancer. Cancer Res. 71, 6764-6772 (2011).

10. Wang, Y. et al. Itraconazole can inhibit malignant pleural effusion by suppressing lymphangiogenesis in mice. Transl. lung cancer Res. 4, 27-35 (2015).

11. Nacev, B. A., Grassi, P., Dell, A., Haslam, S. M. & Liu, J. O. The antifungal drug itraconazole inhibits vascular endothelial growth factor receptor 2 (VEGFR2) glycosylation, trafficking, and signaling in endothelial cells. J. Biol. Chem. 286, 44045-44056 (2011).

12. Ueda, T. et al. Itraconazole Modulates Hedgehog, WNT/beta-catenin, as well as Akt Signalling, and Inhibits Proliferation of Cervical Cancer Cells. Anticancer Res. 37,3521-3526 (2017).

13. Liang, G. et al. Itraconazole exerts its anti-melanoma effect by suppressing Hedgehog, Wnt, and PI3K/mTOR signaling pathways. Oncotarget 8,28510-28525 (2017).

14. Kim, J. et al. Itraconazole, a commonly used antifungal that inhibits Hedgehog pathway activity and cancer growth. Cancer Cell 17, 388-399 (2010).
15. Kim, J. et al. Itraconazole and arsenic trioxide inhibit Hedgehog pathway activation and tumor growth associated with acquired resistance to smoothened antagonists. Cancer Cell 23, 23-34 (2013).

16. Head, S. A. et al. Antifungal drug itraconazole targets VDAC1 to modulate the AMPK/mTOR signaling axis in endothelial cells. Proc. Natl. Acad. Sci. U.S.A. 112, E7276-85 (2015).

17. Xu, J., Dang, Y., Ren, Y. R. & Liu, J. O. Cholesterol trafficking is required for mTOR activation in endothelial cells. Proc. Natl. Acad. Sci. U.S.A. 107, 4764-4769 (2010).

18. Head, S. A. et al. Simultaneous Targeting of NPC1 and VDAC1 by Itraconazole Leads to Synergistic Inhibition of mTOR Signaling and Angiogenesis. ACS Chem. Biol. 12, 174-182 (2017).

19. Kim, D. J. et al. Open-label, exploratory phase II trial of oral itraconazole for the treatment of basal cell carcinoma. J. Clin. Oncol. 32, 745-751 (2014).

20. Rudin, C. M. et al. Phase 2 study of pemetrexed and itraconazole as second-line therapy for metastatic nonsquamous non-small-cell lung cancer. J. Thorac. Oncol. 8, 619-623 (2013).

21. Nussbaum, N. et al. Patient experience in the treatment of metastatic castration-resistant prostate cancer: state of the science. Prostate Cancer Prostatic Dis. 19, 111-121 (2016).

22. Tsubamoto, H., Sonoda, T., Yamasaki, M. & Inoue, K. Impact of combination chemotherapy with itraconazole on survival of patients with refractory ovarian cancer. Anticancer Res. 34, 2481-2487 (2014).

23. Tsubamoto, H., Sonoda, T., Yamasaki, M. & Inoue, K. Impact of combination chemotherapy with itraconazole on survival for patients with recurrent or persistent ovarian clear cell carcinoma. Anticancer Res. 34, 2007-2014 (2014).

24. Piao, S. & Amaravadi, R. K. Targeting the lysosome in cancer. Ann. N.Y. Acad. Sci. 1371, 45-54 (2016).

25. Ballabio, A. & Bonifacino, J. S. Lysosomes as dynamic regulators of cell and organismal homeostasis. Nat. Rev. Mol. Cell Biol. 21, 101-118 (2020).

26. Fennelly, C. & Amaravadi, R. K. Lysosomal Biology in Cancer. Methods Mol. Biol. 1594, 293-308 (2017).

27. Luzio, J. P., Hackmann, Y., Dieckmann, N. M. G. & Griffiths, G. M. The biogenesis of lysosomes and lysosome-related organelles. Cold Spring Harb. Perspect. Biol. 6, a016840 (2014).

28. Wartosch, L., Bright, N. A. & Luzio, J. P. Lysosomes. Curr. Biol. 25, R315-6 (2015).

29. Lu, S., Sung, T., Lin, N., Abraham, R. T. & Jessen, B. A. Lysosomal adaptation: How cells respond to lysosomotropic compounds. PLoS One 12, e0173771 (2017).

30. Bonam, S. R., Wang, F. & Muller, S. Lysosomes as a therapeutic target. Nat. Rev. Drug Discov. 18, 923-948 (2019).

31. Verbaanderd, C. et al. Repurposing Drugs in Oncology (ReDO)-chloroquine and hydroxychloroquine as anti-cancer agents. Ecancermedicalscience 11, 781 (2017).

32. Weber, R. A. et al. Maintaining Iron Homeostasis Is the Key Role of Lysosomal Acidity for Cell Proliferation. Mol. Cell 77, 645-655.e7 (2020).

33. Medrano, M. et al. Interrogation of Functional Cell-Surface Markers Identifies CD151 Dependency in High-Grade Serous Ovarian Cancer. Cell Rep. 18, 2343-2358 (2017).

34. Colic, M. et al. Identifying chemogenetic interactions from CRISPR screens with drugZ. Genome Med. 11, 52 (2019).

35. Schindelin, J. et al. Fiji: an open-source platform for biological-image analysis. Nat. Methods 9, 676-682 (2012).

36. Staunton, J. E. et al. Chemosensitivity prediction by transcriptional profiling. Proc. Natl. Acad. Sci. U.S.A. 98, 10787-10792 (2001).

37. Dong, Z. et al. Anticancer drug sensitivity prediction in cell lines from baseline gene expression through recursive feature selection. BMC Cancer 15, 489 (2015).

38. Rahmati, S., Abovsky, M., Pastrello, C. & Jurisica, I. pathDIP: an annotated resource for known and predicted human gene-pathway associations and pathway enrichment analysis. Nucleic Acids Res. 45, D419-D426 (2017).

39. Ianevski, A., Giri, A. K. & Aittokallio, T. SynergyFinder 2.0: visual analytics of multi-drug combination synergies. Nucleic Acids Res. 48, W488-W493 (2020).

40. McChesney, E. W. Animal toxicity and pharmacokinetics of hydroxychloroquine sulfate. Am. J. Med. 75, 11-18 (1983).

41. Haria, M., Bryson, H. M. & Goa, K. L. Itraconazole. A reappraisal of its pharmacological properties and therapeutic use in the management of superficial fungal infections. Drugs 51, 585-620 (1996).

42. Lee, J. Y. et al. Association of Polymorphisms of Cytochrome P450 2D6 With Blood Hydroxychloroquine Levels in Patients With Systemic Lupus Erythematosus. Arthritis Rheumatol 68, 184-190 (2016).

43. Tett, S., McLachlan, A., Day, R. & Cutler, D. Insights from pharmacokinetic and pharmacodynamic studies of hydroxychloroquine. Agents Actions Suppl 44, 145-190 (1993).

44. Barretina, J. et al. The Cancer Cell Line Encyclopedia enables predictive modelling of anticancer drug sensitivity. Nature 483, 603-607 (2012).

45. Pontano Vaites, L., Paulo, J. A., Huttlin, E. L. & Harper, J. W. Systematic analysis of human cells lacking ATG8 proteins uncovers roles for GABARAPs and the CCZ1/MON1 regulator C18orf8/RMC1 in macro and selective autophagic flux. Mol. Cell. Biol. (2017). doi:10.1128/MCB.00392-17

46. Wei, J. et al. The GARP Complex Is Involved in Intracellular Cholesterol Transport via Targeting NPC2 to Lysosomes. Cell Rep. 19, 2823-2835 (2017).

47. Brown, W. J., Goodhouse, J. & Farquhar, M. G. Mannose-6-phosphate receptors for lysosomal enzymes cycle between the Golgi complex and endosomes. J. Cell Biol. 103, 1235-1247 (1986).

48. Foucquier, J. & Guedj, M. Analysis of drug combinations: current methodological landscape. Pharmacol. Res. Perspect. 3, e00149 (2015).

49. Humphries, W. H. 4th & Payne, C. K. Imaging lysosomal enzyme activity in live cells using self-quenched substrates. Anal. Biochem. 424, 178-183 (2012).

50. Napolitano, G. & Ballabio, A. TFEB at a glance. J. Cell Sci. 129, 2475-2481 (2016).

51. Cook, K. L. et al. Hydroxychloroquine inhibits autophagy to potentiate antiestrogen responsiveness in ER+ breast cancer. Clin. cancer Res. an Off. J. Am. Assoc. Cancer Res. 20, 3222-3232 (2014).

52. Niklaus, M. et al. Expression analysis of LC3B and p62 indicates intact activated autophagy is associated with an unfavorable prognosis in colon cancer. Oncotarget 8,54604-54615 (2017).

53. Tsubamoto, H. et al. Repurposing itraconazole as an anticancer agent. Oncol Lett 14,1240-1246 (2017).

54. de Araujo, M. E. G., Liebscher, G., Hess, M. W. & Huber, L. A. Lysosomal size matters. Traffic 21,60-75 (2020).

55. Chia, P. Z. C. & Gleeson, P. A. The regulation of endosome-to-Golgi retrograde transport by tethers and scaffolds. Traffic 12,939-947 (2011).

56. Zhitomirsky, B. & Assaraf, Y. G. Lysosomes as mediators of drug resistance in cancer. Drug Resist. Updat. 24,23-33 (2016).

57. Zhitomirsky, B. & Assaraf, Y. G. Lysosomal sequestration of hydrophobic weak base chemotherapeutics triggers lysosomal biogenesis and lysosome-dependent cancer multidrug resistance. Oncotarget 6,1143-1156 (2015).

58. Mauthe, M. et al. Chloroquine inhibits autophagic flux by decreasing autophagosome-lysosome fusion. Autophagy 14,1435-1455 (2018).

59. Huss, M. & Wieczorek, H. Inhibitors of V-ATPases: old and new players. J. Exp. Biol. 212,341-346 (2009).

1. A method for preventing or treating cancer in an individual comprising administering a therapeutically effective amount of itraconazole to an individual in need of said treatment, wherein a cell of the individual has a mutation in one or more genes selected from the group consisting of NBEAL1 (SEQ ID No:1), C18orf8 (SEQ ID No:2), COG6 (SEQ ID No:3), SLC30A9 (SEQ ID No:4), ARL1 (SEQ ID No:5), UNC50 (SEQ ID No:6), EPT1 (SEQ ID No:7), RTN4IP1 (SEQ ID No:8), SPTLC2 (SEQ ID No:9), SEC14L1 (SEQ ID No:10), CCDC22 (SEQ ID No:11), SPTSSA (SEQ ID No:12), KIAA1033 (SEQ ID No:13), SPTLC1 (SEQ ID No:14), VPS54 (SEQ ID No:15), TSSC1 (SEQ ID No:16), KIF11 (SEQ ID No:17), NDUFS4 (SEQ ID No:18), COMMD4 (SEQ ID No:19), COL4A3BP (SEQ ID No:20), VPS51 (SEQ ID No:21), VPS52 (SEQ ID No:22) and VPS53 (SEQ ID No:23).

2. The method of claim 1 further comprising assessing or having assessed a cell of the individual to determine whether the cell has a mutation in a gene selected from the group consisting of: NBEAL1 (SEQ ID No:1), C18orf8 (SEQ ID No:2), COG6 (SEQ ID No:3), SLC30A9 (SEQ ID No:4), ARL1 (SEQ ID No:5), UNC50 (SEQ ID No:6), EPT1 (SEQ ID No:7), RTN4IP1 (SEQ ID No:8), SPTLC2 (SEQ ID No:9), SEC14L1 (SEQ ID No:10), CCDC22 (SEQ ID No:11), SPTSSA (SEQ ID No:12), KIAA1033 (SEQ ID No:13), SPTLC1 (SEQ ID No:14), VPS54 (SEQ ID No:15), TSSC1 (SEQ ID No:16), KIF11 (SEQ ID No:17), NDUFS4 (SEQ ID No:18), COMMD4 (SEQ ID No:19), COL4A3BP (SEQ ID No:20), VPS51 (SEQ ID No:21), VPS52 (SEQ ID No:22) and VPS53 (SEQ ID No:23) and selecting the individual for treatment of cancer with itraconazole where the individual has a mutation in a gene selected from the group consisting of: NBEAL1 (SEQ ID No:1), C18orf8 (SEQ ID No:2), COG6 (SEQ ID No:3), SLC30A9 (SEQ ID No:4), ARL1 (SEQ ID No:5), UNC50 (SEQ ID No:6), EPT1 (SEQ ID No:7), RTN4IP1 (SEQ ID No:8), SPTLC2 (SEQ ID No:9), SEC14L1 (SEQ ID No:10), CCDC22 (SEQ ID No:11), SPTSSA (SEQ ID No:12), KIAA1033 (SEQ ID No:13), SPTLC1 (SEQ ID No:14), VPS54 (SEQ ID No:15), TSSC1 (SEQ ID No:16), KIF11 (SEQ ID No:17), NDUFS4 (SEQ ID No:18), COMMD4 (SEQ ID No:19), COL4A3BP (SEQ ID No:20), VPS51 (SEQ ID No:21), VPS52 (SEQ ID No:22) and VPS53 (SEQ ID No:23).

3. The method of claim 2 wherein the mutation is a loss of function mutation.

4. The method of claim 3 comprising determining whether the cell of the individual has reduced production, or no production of a protein or RNA encoded by a gene selected from the group consisting of: NBEAL1 (SEQ ID No:1), C18orf8 (SEQ ID No:2), COG6 (SEQ ID No:3), SLC30A9 (SEQ ID No:4), ARL1 (SEQ ID No:5), UNC50 (SEQ ID No:6), EPT1 (SEQ ID No:7), RTN4IP1 (SEQ ID No:8), SPTLC2 (SEQ ID No:9), SEC14L1 (SEQ ID No:10), CCDC22 (SEQ ID No:11), SPTSSA (SEQ ID No:12), KIAA1033 (SEQ ID No:13), SPTLC1 (SEQ ID No:14), VPS54 (SEQ ID No:15), TSSC1 (SEQ ID No:16), KIF11 (SEQ ID No:17), NDUFS4 (SEQ ID No:18), COMMD4 (SEQ ID No:19), COL4A3BP (SEQ ID No:20), VPS51 (SEQ ID No:21), VPS52 (SEQ ID No:22) and VPS53 (SEQ ID No:23), wherein reduced production or no production of a protein or RNA encoded by a gene selected from said group determines that the cell has a loss of function mutation in the gene, thereby determining whether the cell has a loss of function mutation in a gene of said group.

5. The method of claim 2 wherein the cell that is assessed for a mutation is a cancer cell.

6. The method of any one of claim 2 wherein the cancer is an epithelial cancer.

7. The method of claim 2 wherein the cancer is ovarian cancer.

8. The method of claim 7 wherein the cancer is refractory or resistant ovarian cancer.

9. The method of claim 7 wherein an ovarian cancer cell is assessed to determine whether the cancer cell contains a loss of function mutation in C18orf8 (SEQ ID No:2) and/or VPS54 (SEQ ID No:15).

10. The method of claim 2 wherein itraconazole is utilised as a monotherapy for treatment of the cancer.

11. The method of claim 2 wherein itraconazole is utilised as a chemosensitizer for a further chemotherapeutic compound for treatment of the cancer.

12. The method of claim 11 wherein the further chemotherapeutic compound is selected from the group consisting of chloroquine and tamoxifen.
